(19) United States
(12) Patent Application Publication
(10) Pub. No.: US 2008/0279893 A1
(43) Pub. Date: Nov. 13, 2008

(54) Lawsonia intracellularis immunological proteins

(75) Inventors: Eric Vaughn, Ames, IA (US); Merrill Schaeffer, St. Joseph, MO (US); Yajie Liang, Ames, IA (US)

Correspondence Address: Tracey S. Truitt;Erickson & Kleypas, L.L.C., 800 W. 47th Street, Suite 401, Kansas City, MO, 64112, US

(73) Assignee: Boehringer Ingelheim Vetmedica, Inc.

(21) Appl. No.: 11/414,764

(22) Filed: Apr. 28, 2006

Related U.S. Application Data
(60) Provisional application No. 60/675,806, filed on Apr. 28, 2005.

(51) Int. Cl. A61K 39/00; C07K 14/00; C12N 15/11; C12N 15/00; G01N 33/569; A61K 31/70; A61K 38/00

(52) U.S. Cl. 424/234.1; 530/350; 536/23.7; 435/320.1; 514/44; 514/12; 435/7.32

(57) ABSTRACT

The present invention provides nucleic acid and amino acid sequences useful as the immunogenic portion of vaccines or immunogenic compositions effective for lessening the severity of the clinical symptoms associated with Lawsonia intracellularis infection or conferring protective immunity to an animal susceptible to such infection. Preferred amino acid sequences are selected from the group consisting of 1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466; 2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1); 3) any immunogenic portion of the polypeptides of 1) and/or 2) 4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or 5) a polypeptide that is encoded by a DNA that codes for a peptide comprising the sequence of SEQ ID No: 1-455 or SEQ ID No: 466. Thus, the nucleic acid sequences encoding such proteins, or the proteins themselves are included in vaccine compositions, together with veterinary-acceptable carrier and administered to an animal in need thereof.

### RELATED APPLICATIONS

This application claims the benefit of provisional application Ser. No. 60/675,806, filed on Apr. 28, 2005, the teachings and contents of which are hereby incorporated by reference.

### SEQUENCE LISTING

This application contains a sequence listing in computer readable format, the teachings and content of which are hereby incorporated by reference.

### BACKGROUND OF THE INVENTION

1. Field of the Invention

The present application is concerned with antigens of Lawsonia intracellularis and their use. More particularly, the present application is concerned with antigens that are immunologically relevant proteins and the nucleic acid sequences or DNA molecules encoding those proteins and vectors including DNA molecules coding for immunological proteins of Lawsonia intracellularis. Even more particularly, the present invention is concerned with the identification of such proteins and nucleic acid sequences. Still more particularly, the present invention is concerned with determining whether such proteins or nucleic acid sequences are good candidates for use in a subunit vaccine by their location. Even more particularly, the present invention is concerned with such proteins and nucleic acid sequences that are capable of invoking an immune response in a host animal. Still more particularly, the present application is concerned with such proteins and nucleic acid sequences and their incorporation into an immunogenic composition as well as the subsequent administration of such a composition to a host animal. The proteins and/or nucleic acid sequences can be used as a component in a vaccine and the vaccine used to provide a degree of protective immunity against and/or a lessening of the clinical symptoms associated with infection by Lawsonia intracellularis. The present application is also concerned with methods of producing and administering vaccines comprising such nucleic acid sequences or the proteins encoded thereby. Finally, the present application is concerned with diagnostic tests for the detection of Lawsonia intracellularis as well as methods of producing and administering vaccine incorporating such Lawsonia intracellularis antigens.

2. Description of the Prior Art

Lawsonia Intracellularis is the causative agent of porcine proliferative interopathy (“PPE”), and it effects virtually all animals, including humans, rabbits, ferrets, hamsters, fox, horses, and other animals as diverse as ostriches and emus. PPE is a common diarrheal disease of growing-finishing and young breeding pigs characterized by hyperplasia and inflammation of the ileum and colon. It often is mild and self-limiting but sometimes causes persistent diarrhea, depression, reduced appetite, reluctance to move, retarded growth, increased FCR, severe necrotic enteritis, or hemorrhagic enteritis with high mortality. The bacteria itself is an obligate, intracellular bacterium.

The bacteria associated with PPE have been referred to as “Campylobacter-like organisms.” S. McOrist et al., Vet. Pathol., Vol. 26, 260-264 (1989). Subsequently, the causative bacteria have been identified as a novel taxonomic genus and species, vernacularly referred to as Ileal symbiont (IS) intracellularis. C. Gebhart et al., Int'l. J. of Systemic Bacteriology, Vol. 43, No. 3, 533-538 (1993). More recently, these novel bacteria have been given the taxonomic name Lawsonia (L.) intracellularis. S. McOrist et al., Int'l. J. of Systemic Bacteriology, Vol. 45, No. 4, 820-825 (1995). These three names have been used interchangeably to refer to the same organism as further identified and described herein. Koch's postulates have been fulfilled by inoculation of pure cultures of L intracellularis into conventionally reared pigs; typical lesions of the disease were produced, and L intracellularis was reisolated from the lesions. The more common, nonhemorrhagic form of the disease often affects 18- to 36-kg pigs and is characterized by sudden onset of diarrhea. The feces are watery to pasty, brownish, or faintly blood stained. After ˜2 days, pigs may pass yellow fibrinonecrotic casts that have formed in the ileum. Most affected pigs recover spontaneously, but a significant number develop chronic necrotic enteritis with progressive emaciation. The hemorrhagic form is characterized by cutaneous pallor, weakness, and passage of hemorrhagic or black, tarry feces. Pregnant gilts may abort. Lesions may occur anywhere in the lower half of the small intestine, cecum, or colon but are most frequent and obvious in the ileum. The wall of the intestine is thickened, and the mesenterymaybe edematous. The mesenteric lymph nodes are enlarged. The intestinal mucosa appears thickened and rugose, may be covered with a brownish or yellow fibrinonecrotic membrane, and sometimes has petechial hemorrhages. Yellow necrotic casts may be found in the ileum or passing through the colon. Diffuse, complete mucosal necrosis in chronic cases causes the intestine to be rigid, resembling a garden hose. Proliferative mucosal lesions often are in the colon but are detected only by careful inspection at necropsy. In the profusely hemorrhagic form, there are red or black, tarry feces in the colon and clotted blood in the ileum. Altogether, L. intracellularis is a particularly great cause of losses in swine herds in Europe as well as in the United States.

L. intracellularis is an obligate, intracellular bacterium which cannot be cultured by normal bacteriological methods on conventional cell-free media and has been thought to require cells for growth. S. McOrist et al., Infection and Immunity, Vol. 61, No. 19, 4286-4292 (1993) and G. Lawson et al., J. of Clinical Microbiology, Vol. 31, No. 5, 1136-1142 (1993) discuss cultivation of L. intracellularis using IEC-18 rat intestinal epithelial cell monolayers in conventional tissue culture flasks. In U.S. Pat. Nos. 5,714,375 and 5,885,823, both of which are herein incorporated by reference in their entireties, cultivation of L. intracellularis in suspended host cells was described.

Pathogenic and non-pathogenic attenuated bacteria strains of L. intracellularis are well known in state of the art. For example, WO 96/39629 and WO 05/011731 describe non-pathogenic attenuated strains of L. intracellularis. Further attenuated bacteria strains of L. intracellularis are known from WO 02/26250 and WO 03/00665.

What is needed in the art is a vaccine effective against Lawsonia intracellularis infection, which provides or confers protective immunity to an animal and/or reduces the severity of clinical symptoms associated with Lawsonia intracellularis infection. What is further needed are methods of making and administering such vaccines.

### SUMMARY OF THE INVENTION

The present invention overcomes the problems inherent in the prior art and provides a distinct advance in the state of the art. Specifically, this invention concerns antigens comprising immunological proteins derived from Lawsonia intracellularis and their use in the vaccination of swine against infection by Lawsonia intracellularis. Preferably, the proteins will elicit a humoral immune response during the normal course of infection in swine. These proteins, both individually and in combination, will be useful as a component in a protein subunit vaccine that invokes an immune response and provides protective immunity against or a lessening of the clinical symptoms associated with Lawsonia intracellularis infection. The identified proteins can then be generated by any conventional means and used in a vaccine.

The Lawsonia intracellularis DK15540 genomic nucleotide sequence was analyzed for the presence of nucleotide sequences that would encode proteins having a minimum length of 300 amino acids. Altogether, 456 protein sequences having at least 300 amino acids were identified. These sequences corresponded to SEQ ID Nos. 1-455 and 466. These protein sequences were further analyzed using two separate computer programs, PSORT and CELLO. The purpose of this analysis was to identify proteins of interest that were 300 amino acids or longer, and find or predict their location in Lawsonia intracelluaris. Knowledge of the location of a protein will indicate the suitability of a protein for use in a subunit vaccine to one of skill in the art. The PSORT program is used to predict subcellular localization and is hosted by the Brinkman Laboratory at Simon Fraser University and can be found at psort.org. The CELLO program uses a Support Vector Machine based on n-peptide composition to assign a Gram-negative protein to the cytoplasm, inner membrane, periplasm, outer membrane or extracellular space and is found at cello.life.nctu.edu.tw. Generally, the suitability of a protein as a component in a subunit vaccine is, in increasing order of suitability, cytoplasmic, inner membrane, periplasmic, outer membrane, and extracellular. In other words, extracellular proteins provide the greatest likelihood of effectiveness for vaccines, while cytoplasmic proteins provide the least likelihood of suitability. This is because such proteins are more exposed and accessible for the inducement of an immune response. Using CELLO, extracellular proteins included SEQ ID Nos. 6, 329, 296, 413, 194, 143, 146, 333, 438, 188, 261, 237, 336, 291, 151, 26, 139, 333, 444, 308, 131, 284, and 340, or an immunogenic portion thereof; outer membrane proteins included SEQ ID Nos. 355, 11, 378, 50, 35, 231, 4, 328, 313, 27, 172, 275, 387, 134, 201, 256, 2, 12, 404, 388, 327, 306, 415, 343, 373, 214, 330, 316, 428, 190, 129, 320, 381, 9, 292, 158, 270, 336, 423, 211, 178, 430, 77, 186, 264, 140, 193, 192, 208, 183, 108, 109, 87, 253, 379, 243, 364, 51, 99, 419, 278, 295, 349, 219, 127, 389, 254, 263, 294, 315, 257, 443, 403, 76, 75, 73, 344, 74, and 238, or an immunogenic portion thereof; periplasmic proteins included SEQ ID Nos. 6,132,421, 112, 110, 310, 247, 205, and 7, or an immunogenic portion thereof; inner membrane proteins included SEQ ID Nos. 228, 452, 144, 323, 305, 357, 360, 95, 130, 34, 405, 118, 451, 299, 48, 376, 358, 377, 352, 39, 106, 258, 309, 445, 195, 311, 179, 410, 265, 249, 354, 398, 408, 20, 44, 68, 31, 153, 187, 345, 69, 366, 348, 1, 324, 281, 88, 239, 36, 276, 29, 104, 70, 426, 302, 314, 369, 418, 58, 166, 384, 107, 18, 272, 41, 200, 180, 92, 386, 156, 455, 383, 361, 116, 277, 55, 252, 32, 93, 241, 120, 229, 121, 89, 382, and 250, or an immunogenic portion thereof; and cytoplasmic proteins included SEQ ID Nos. 6, 79, 346, 332, 11, 53, 81, 8, 21, 435, 234, 185, 450, 5347, 424, 326, 155, 215, 399, 209, 216, 416, 147, 313, 157, 342, 343, 293, 271, 337, 72, 269, 103, 64, 425, 148, 341, 24, 285, 289, 429, 268, 177, 405, 260, 407, 100, 442, 321, 370, 47, 353, 80, 67, 436, 30, 220, 397, 212, 96, 149, 119, 273, 105, 85, 15, 3, 232, 40, 225, 420, 19, 286, 259, 196, 207, 176, 280, 431, 160, 367, 168, 128, 124, 394, 126, 5, 255, 242, 46, 152, 16, 65, 433, 167, 221, 414, 287, 412, 111, 303, 449, 114, 233, 406, 25, 210, 61, 203, 86, 141, 171, 447, 266, 437, 173, 78, 199, 319, 400, 392, 351, 184, 43, 217, 189, 174, 409, 204, 396, 83, 335, 98, 224, 113, 372, 301, 164, 246, 56, 175, 262, 226, 17, 362, 338, 267, 35.6, 25.1, 300, 62, 14, 350, 37, 202, 159, 115, 331, 317, 163, 38, 240, 318, 236, 304, 439, 191, 244, 97, 417, 133, 123, 22, 359, 165, 385, 218, 162, 102, 223, 283, 453, 290, 402, 71, 446, 380, 339, 122, 161, 117, 390, 82, 427, 371, 454, 49, 368, 28, 10, 42, 63, 57, 59, 54, 136, 84, 181, 60, 90, 52, 125, 230, 142, 440, 197, 363, 23, 325, 154, 227, 282, 213, 33, 391, 91, 312, 198, 101, 45, 422, 298, 448, 375, 274, 150, 206, 374, 248, 393, 222, 288, 235, 66, 182, 307, 334, 322, 169, 279, 13, 395, 434, 365, 137, 145, 170, 401, 441, 138, 94, 245, 411, and 135, or an immunogenic portion thereof. Moreover, the order provided in each of the CELLO prediction lists above provides the proteins in order, from least suitable of the group to most suitable, for vaccine purposes. Thus, for purposes of the present invention, it is preferred to use a Lawsonia intracellularis protein. More preferably, it is preferred to use a sequence selected from the group consisting of SEQ ID Nos. 1-455 and 466, as well as the proteins encoded by SEQ ID Nos. 456 and 457. Still more preferably, it is preferred to use an extracellular or outer membrane protein, and even more preferably, a protein selected from the group consisting of SEQ ID Nos. 355, 11, 378, 50, 35, 231, 4, 328, 313, 27, 172, 275, 387, 134, 201, 256, 2, 12, 404, 388, 327, 306, 415, 343, 373, 214, 330, 316, 428, 190, 129, 320, 381, 9, 292, 158, 270, 336, 423, 211, 178, 430, 77, 186, 264, 140, 193, 192, 208, 183, 108, 109, 87, 253, 379, 243, 364, 51, 99, 419, 278, 295, 349, 219, 127, 389, 254, 263, 294, 315, 257, 443, 403, 76, 75, 73, 344, 74, 238, 6, 329, 296, 413, 194, 143, 146, 333, 438, 188, 261, 237, 336, 291, 151, 26, 139, 333, 444, 308, 131, 284, and 340, or any immunogenic portion, or homolog of the above-mentioned Lawsonia proteins, or any immunogenic portion of said homolog. Again, these proteins are listed in order of increasing suitability for use in a subunit vaccine. Still more preferably, extracellular proteins are used, and even more preferably, the protein is selected from the group consisting of SEQ ID Nos. 6, 329, 296, 413, 194, 143, 146, 333, 438, 188, 261, 237, 336, 291, 151, 26, 139, 333, 444, 308, 131, 284, and 340, or any immunogenic portion, or homolog of the above-mentioned Lawsonia proteins, or any immunogenic portion of said homolog. The complete CELLO results are included in Table 1 of U.S. Ser. No. 60/675,806, the application to which benefit is claimed herein.

Using PSORT, extracellular proteins (ECSVM—Localization) included SEQ ID Nos. 237, 292, and 327; outer membrane proteins (OMSVM—Localization) included SEQ ID Nos. 51, 108, 140, 193, 194, 211, 217, 219, 237, 256, 257, 269, 278, 284, 292, 294, 315, 327, 329, 344, 349, 389, and 403; outer membrane proteins identified by Motif—Localization included SEQ ID Nos. 32, 70, and 155; no periplasmic proteins were identified using PPSVM—Localization; periplasmic proteins identified using Motif-Localization included 187, 250, 272, and 303; inner membrane proteins identified by CMSVN—Localization included SEQ ID Nos. 1, 16, 18, 20, 29, 31, 32, 41, 44, 55, 58, 68, 69, 70, 88, 89, 92, 93, 104, 107, 116, 120, 121, 153, 156, 166, 179, 180, 187, 195, 200, 229, 239, 241, 250, 252, 272, 276, 277, 300, 302, 314, 324, 345, 348, 361, 366, 369, 382, 383, 384, 386, 408, 410, 418, 426, 432, and 455; inner membrane proteins identified using HMMTOP—Localization included SEQ ID Nos. 16, 18, 20, 29, 31, 32, 36, 41, 44, 53, 55, 58, 67, 68, 69, 70, 74, 77, 88, 89, 92, 93, 104, 107, 114, 116, 120, 121, 140, 146, 153, 156, 166, 179, 180, 187, 195, 200, 201, 211, 229, 239, 241, 242, 250, 252, 265, 272, 276, 277, 278, 281, 292, 302, 310, 311, 314, 324, 341, 345, 348, 354, 355, 361, 366, 369, 382, 383, 384, 386, 404, 408, 410, 418, 424, 426, 427, 432, 443, and 455; and cytoplasmic proteins identified using CytoSVM—Localization included SEQ ID Nos. 5, 8, 10, 13, 17, 22, 23, 24, 30, 33, 37, 38, 42, 43, 45, 49, 52, 54, 60, 62, 63, 64, 84, 85, 86, 90, 91, 94, 98, 101, 113, 125, 133, 135, 136, 137, 138, 142, 145, 150, 152, 154, 155, 165, 168, 169, 170, 171, 173, 174, 175, 176, 181, 182, 189, 197, 198, 202, 206, 213, 214, 218, 220, 221, 222, 223, 224, 226, 227, 230, 235, 236, 240, 242, 245, 247, 248, 254, 255, 268, 274, 279, 282, 288, 293, 295, 298, 303, 304, 307, 312, 313, 317, 325, 330, 334, 338, 350, 352, 353, 356, 362, 363, 365, 368, 371, 372, 374, 375, 380, 385, 390, 392, 394, 395, 400, 401, 402, 406, 407, 409, 411, 412, 417, 420, 422, 431, 433, 434, 437, 439, 440, 441, 443, 448, 453, and 454. The complete PSORT results were provided in Table 2 of U.S. Ser. No. 60/675,806.

Next, each of the sequences were searched through BLAST in order to find other proteins that were homologous to the 456 Lawsonia proteins. The results of this BLAST searching is contained herein as FIG. 5.

Finally, amino acid alignments between the Lawsonia DK15540 hemolysin and Omp85-like proteins with Desulfovibria were provided in TABLE 3 of U.S. Ser. No. 60/675,806.

As used herein, the following definitions will apply: “Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95% “sequence identity” to a reference nucleotide sequence, it is intended that the nucleotide sequence of the given polynucleotide is identical to the reference sequence except that the given polynucleotide sequence may include up to 5 point mutations per each 100 nucleotides of the reference nucleotide sequence. In other words, in a polynucleotide having a nucleotide sequence having at least 95% identity relative to the reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95% sequence identity to a reference amino acid sequence, it is intended that the given amino acid sequence of the polypeptide is identical to the reference sequence except that the given polypeptide sequence may include up to 5 amino acid alterations per each 100 amino acids of the reference amino acid sequence. In other words, to obtain a given polypeptide sequence having at least 95% sequence identity with a reference amino acid sequence, up to 5% of the amino acid residues in the reference sequence may be deleted or substituted with another amino acid, or a number of amino acids up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

Similarly, “sequence homology”, as used herein, also refers to a method of determining the relatedness of two sequences. To determine sequence homology, two or more sequences are optimally aligned as described above, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 95% of the amino acid residues or nucleotides in the reference sequence must match or comprise a conservative substitution with another amino acid or nucleotide, or a number of amino acids or nucleotides up to 5% of the total amino acid residues or nucleotides, not including conservative substitutions, in the reference sequence may be inserted into the reference sequence.

A “conservative substitution” refers to the substitution of an amino acid residue or nucleotide with another amino acid residue or nucleotide having similar characteristics or properties including size, hydrophobicity, etc., such that the overall functionality does not change significantly.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

In general, each sequence described herein including the protein sequences and the DNA encoding such proteins also covers proteins and DNA sequences having certain percentages of sequence homology or sequence identity relative to the disclosed sequences. While it is preferred to have high percentages of sequence homology or identity, it is more preferred to retain the functions of the claimed sequence than the sequence per se. In other words, those of skill in the art will be able to make minor changes to the sequences disclosed herein yet retain the functionality of the disclosed sequences with such “derivative” sequences. Conservative substitutions would be one preferred method of making changes to the sequence while still preserving functionality. Preferably the present invention will embrace other sequences including derivative sequences that are based on the sequences disclosed herein. Such other sequences will preferably have at least about 85% sequence identity or homology, more preferably at least about 90% sequence identity or homology, still more preferably at least about 95% sequence identity or homology, even more preferably at least about 97% sequence identity or homology, still even more preferably at least about 98% sequence identity or homology, and even more preferably at least about 99% sequence identity or homology with a sequence disclosed herein. Preferably, such homology exists over a lengths of at least 25 amino acids/nucleotides, more preferably at least 50 amino acids/nucleotides, even more preferably at least 75 amino acids/nucleotides, still even more preferably at least 150 amino acids/nucleotides, even more preferably at least 200 amino acids/nucleotides, even more preferably at least 250 amino acids/nucleotides, and most preferably, at least 300 amino acids/nucleotides.

Additionally, it is understood that the protein sequences described herein are useful in immunogenic compositions and that some stretches or portions of these sequences play a greater role in inducing an immune response than others. This means that sufficient immune responses could be induced by using just selected portions of these proteins, provided that the selected portions were of sufficient length to generate an immune response. Accordingly, the invention covers any immunogenic portion of the proteins described herein. Moreover, the invention also covers any DNA molecules encoding for those immunogenic stretches or portions. Generally, such stretches or portions will comprise the sequence of contiguous amino acids/nucleotides up to the entire length of the sequence. More preferably, such stretches or portions will, in ascending order of preference, have at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, 9, or most preferably 8 contiguous amino acids from the disclosed sequence, or any homolog thereof. When related to a DNA molecule, such stretches or portions will, in ascending order of preference, encoding for at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, 9, or most preferably 8 contiguous amino acids from the disclosed sequence. Preferably, said homolog sequences will preferably have at least about 85% sequence identity or homology, more preferably at least about 90% sequence identity or homology, still more preferably at least about 95% sequence identity or homology, even more preferably at least about 97% sequence identity or homology, still even more preferably at least about 98% sequence identity or homology, and even more preferably at least about 99% sequence identity or homology with a sequence disclosed herein. As with the sequences themselves, such stretches are also operable for manipulation without loss of function by those of skill in the art. Accordingly, the sequence homology and sequence identity definitions also apply to these stretches or portions of the disclosed proteins.

As used herein, the term “L. intracellularis” or “Lawsonia intracellularis” or “Lawsonia” means the intracellular, curved gram-negative bacteria described in detail by C. Gebhart et al., Int'l. J. of Systemic Bacteriology, Vol. 43, No. 3, 533-538 (1993) and S. McOrist et al., Int'l. J. of Systemic Bacteriology, Vol. 45, No. 4, 820-825 (1995), each of which is incorporated herein by reference in their entireties, and includes but is not limited to the isolates described in WO 96/39629 and WO 05/011731. In particular, the term “L. intracellularis” also means, but is not limited to the isolates deposited under the Budapest Treaty with the American Type Culture Collection, 10801 University Boulevard, Manassas, Va. 20110-2209 and assigned ATCC accession number PTA 4926 or ATCC accession number 55783. Both isolates are described in WO 96/39629 and WO 05/011731, respectively. The term “L. intracellularis” also means, but is not limited to any other L. intracellularis bacteria strain or isolate preferably having the immunogenic properties of at least one of the L. intracellularis strains described in WO 96/39629 and WO 05/011731, in particular having the immunogenic properties of at least one of the isolates deposited under the Budapest Treaty with the American Type Culture Collection, 10801 University Boulevard, Manassas, Va. 20110-2209 and assigned ATCC accession numbers PTA 4926 or ATCC accession number 55783.

Moreover, the term “L. intracellularis” also means any L. intracellularis antigen. The term “L. intracellularis antigen” as used herein means, but is not limited to any composition of matter, that comprises at least one antigen that can induce, stimulate or enhance the immune response against a L. intracellularis-caused infection, when administered to an animal, preferably a pig. Preferably, said L. intracellularis antigen is a complete L. intracellularis bacterium, in particular in an inactivated form (a so called killed bacterium), a modified live or attenuated L. intracellularis bacterium (a so called MLB), a chimeric vector that comprises at least an immunogenic amino acid sequence of L. intracellularis, or any other polypeptide or component, that comprises at least an immunogenic amino acid sequence of L. intracellularis. The terms “immunogenic protein”, “immunogenic polypeptide” or “immunogenic amino acid sequence” as used herein, refer to any amino acid sequence which elicits an immune response in a host against a pathogen comprising said immunogenic protein, immunogenic polypeptide or immunogenic amino acid sequence. In particular, an “immunogenic protein”, “immunogenic polypeptide” or “immunogenic amino acid sequence” of L. intracellularis means any amino acid sequence that codes for an antigen which elicits an immunological response against L. intracellularis in a host to which said “immunogenic protein”, “immunogenic polypeptide” or “immunogenic amino acid sequence” is administered. For example, the proteins having the sequences of SEQ ID Nos 1-455 and SEQ ID No 466, or any immunogenic portion thereof are considered to be an “immunogenic protein”, “immunogenic polypeptide” or “immunogenic amino acid sequence” of Lawsonia intracellularis. Furthermore, these terms include, but are not limited to the full-length sequence of any proteins, analogs thereof, or immunogenic fragments or portions thereof. The term “immunogenic fragment” or “immunogenic portion” means a fragment of a protein which includes one or more epitopes and thus elicits the immunological response against the relevant pathogen. Such fragments can be identified using any number of epitope mapping techniques that are well known in the art. See, e.g., Epitope Mapping Protocols in Methods in Molecular Biology, Vol. 66 (Glenn E. Morris, Ed., 1996) Humana Press, Totowa, N.J. (The teachings and content of which are incorporated by reference herein.) For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. (The teachings and content of which are incorporated by reference herein.) Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998. (The teachings and content of which are incorporated by reference herein.)

A strain or isolate has the “immunogenic properties” of at least one of the L. intracellularis strains described in WO 96/39629 and WO 05/011731, in particular, of the isolates deposited as ATCC accession number PTA 4926 or ATCC accession number 55783, when it is detectable at least with one of the anti-L. intracellularis specific antibodies, described in WO06/01294, in a detection assay that is also described in WO06/01294. Preferably those antibodies are selected from the antibodies having the reference numbers 301:39, 287:6, 268:29, 110:9, 113:2 and 268:18. Preferably, the detection assay is a sandwich ELISA as described in Examples 2 and 3 of WO06/12949, whereas antibody 110:9 is used as an capture antibody and antibody 268:29 is used as conjugated antibody. All antibodies disclosed in WO06/12949 are produced by hybridoma cells, which are deposited at the Centre for Applied Microbiology and Research (CAMR) and European Collection of Cell Cultures (ECACC)”, Salisbury, Wiltshire SP4 0JG, UK, as a patent deposit according to the Budapest Treaty. The date of deposit was May 11, 2004. HYBRIDOMA CELL LINE 110:9 is successfully deposited under ECACC Acc. No. 04092204. HYBRIDOMA CELL LINE 113:2 is successfully deposited under ECACC Acc. No. 04092201. HYBRIDOMA CELL LINE 268:18 is successfully deposited under ECACC Acc. No. 04092202. HYBRIDOMA CELL LINE 268:29 is successfully deposited under ECACC Acc. No. 04092206. HYBRIDOMA CELL LINE 287:6 is successfully deposited under ECACC Acc. No. 04092203. HYBRIDOMA CELL LINE 301:39 is successfully deposited under ECACC Acc. No. 04092205.

Several of the sequences comprising the genome of Lawsonia intracellularis have been described in PCT applications WO0069903, WO0069904, WO0069905, and WO0069906 as well as European Patent 1094070, all of which have their teachings and contents incorporated by reference herein.

An “immunological response” or “immune response” to a composition or vaccine is the development in the host of a cellular and/or antibody-mediated immune response to the composition or vaccine of interest. Usually, an “immune response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of the symptoms associated with host infections as described above.

In addition, the immunogenic and vaccine compositions of the present invention can include one or more veterinary-acceptable carriers. As used herein, “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997). For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Cabopol 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferred the adjuvant is added in an amount of about 100 μg to about 10 mg per dose. Even more preferred the adjuvant is added in an amount of about 500 μg to about 5 mg per dose. Even more preferred the adjuvant is added in an amount of about 750 μg to about 2.5 mg per dose. Most preferably, the adjuvant is added in an amount of about 1 mg per dose.

Owing to the degeneracy of the genetic code, it is known that several variations of nucleic acids may encode the same protein. As the encoding of amino acids and the genetic code are both well known in the art, all such variations in nucleic acid sequences that result in the same amino acid are covered by the present invention.

In one embodiment of the present invention, there is provided an immunological protein derived from Lawsonia intracellularis. It is herewith understood, that the terms “immunogenic and “immunological” are synonymously used herein. Preferably, the protein is selected from the group consisting of Lawsonia proteins. More preferably, the immunological protein is coded for by a DNA sequence coding for a protein having at least 85%, more preferably 90%, still more preferably 93%, even more preferably 95%, still more preferably 97%, even more preferably 98%, still more preferably 99% and most preferably 100% sequence homology with a sequence selected from the group consisting of SEQ ID Nos. 1-455 and 466 and combinations thereof. Alternatively, the protein is encoded for by a DNA sequence having at least about 85%, more preferably 90%, still more preferably 93%, even more preferably 95%, still more preferably 97%, even more preferably 98%, still more preferably 99% and most preferably 100% sequence homology with a sequence selected from the group consisting of SEQ ID No. 456 and SEQ ID No. 457. More preferably, the protein is selected from the group consisting of extracellular and outer membrane Lawsonia proteins. Still more preferably, the protein is selected from the group consisting of SEQ ID Nos. 355, 11, 378, 50, 35, 231, 4, 328, 313, 27, 172, 275, 387, 134, 201, 256, 2, 12, 404, 388, 327, 306, 415, 343, 373, 214, 330, 316, 428, 190, 129, 320, 381, 9, 292, 158, 270, 336, 423, 211, 178, 430, 77, 186, 264, 140, 193, 192, 208, 183, 108, 109, 87, 253, 379, 243, 364, 51, 99, 419, 278, 295, 349, 219, 127, 389, 254, 263, 294, 315, 257, 443, 403, 76, 75, 73, 344, 74, 238, 6, 329, 296, 413, 194, 143, 146, 333, 438, 188, 261, 237, 336, 291, 151, 26, 139, 333, 444, 308, 131, 284, 340, 466, and combinations thereof. Still more preferably, the protein is selected from the group consisting of SEQ ID Nos. 344, 466, and combinations thereof. It is furthermore understood that the reference to the sequences of SEQ ID NOS 1-455 as used herein, includes the reference to each individual sequence, which means for example to SEQ ID No 1, No. 2, No. 3, No. 4, No. 5, . . . , No. 450, No. 451, No. 452, No. 453, No. 454, and No. 455. More preferably, the immunological protein or combination of proteins reacts with convalescent swine serum in a Western blot. In another embodiment of the present invention, the immunological protein has a similar function and/or generates a similar immune response as a protein coded by either SEQ ID No. 456 or SEQ ID No. 457 or a protein selected from the group consisting of SEQ ID Nos. 1-455 and 466 (e.g. a “reference protein”). To “generate a similar immune response as a reference protein coded by either SEQ ID No. 456 or SEQ ID No. 457 or a protein selected from the group consisting of SEQ ID Nos. 1-455 and 466” as used herein, means that the immunological protein reacts in a standardized detection assay, e.g. an ELISA, with an amplitude of at least 20%, preferably 50%, even more preferred 75%, most preferred 100% as compared to the amplitude detected for the corresponding reference protein, when used in the detection assay under the same conditions. It being further understood that a combination of proteins may induce a greater immune response and thereby provide greater protective immunity than a single protein.

Another embodiment of the present invention provides an immunogenic protein or a vaccine composition comprising an amino acid sequence having at least 8 contiguous amino acids from a protein sequence as described above, homologs or immunogenic portions thereof, or homologs of said immunogenic portions. Still more preferably, the amino acid sequence which includes the required contiguous amino acids will be up to 8 amino acids in length, more preferably, up to 14 amino acids in length, still more preferably up to 23 amino acids in length, even more preferably, up to 40 amino acids in length, still more preferably, at least up to 70 amino acids in length, and still more preferably, up to 100 amino acids in length, still more preferably up to 200 amino acids in length, and even more preferably up to 300 amino acids in length. In preferred forms, the immunogenic or vaccine composition of the present invention will further comprise veterinary-acceptable carriers, as set forth above.

In another embodiment of the present invention, there is provided a method of vaccinating animals, preferably swine by inoculating them with an immunological protein derived from Lawsonia intracellularis. Preferably, the protein is as described above.

In another embodiment of the present invention, the vaccine comprises proteins selected from the group consisting of any one of SEQ ID Nos. 1-455 and 466, the protein encoded by SEQ ID No. 456, the protein encoded by SEQ ID No. 457, proteins that have similar functions and induce similar immune responses as any one of SEQ ID Nos. 1-455 and 466, or any portion thereof, proteins that have similar functions and induce similar immune responses to the protein encoded by SEQ ID No. 456, proteins that have similar functions and induce similar immune responses as the protein encoded by SEQ ID No. 457, immunogenic portions thereof, and combinations thereof.

In another embodiment of the present invention, the animals are vaccinated by inoculating them with a vaccine prepared by inserting DNA coding for an immunological protein derived from Lawsonia intracellularis into a vector and administering the vector through any conventional means. One preferred method of administration is oral. Preferably, the vector is a bacteria. More preferably, the vector is salmonella. Preferably, the protein is selected from the group consisting of Lawsonia proteins. More preferably, the protein coded for by the DNA is selected from the group consisting of SEQ ID Nos. 1-455 and 466, homologs thereof, immunogenic portions thereof, homologs of said immunogenic portions, proteins that have similar functions and induce similar immune responses as any one of SEQ ID Nos. 1-455 and 466, proteins that have similar functions and induce similar immune responses to the protein encoded by SEQ ID NO. 456, proteins that have similar functions and induce similar immune responses as the protein encoded by SEQ ID No. 457, and combinations thereof. More preferably, the immunological protein is coded for by a DNA sequence coding for a protein having at least 85%, more preferably 90%, still more preferably 93%, even more preferably 95%, still more preferably 97%, even more preferably 98%, still more preferably 99% and most preferably 100% sequence homology with a sequence selected from the group consisting of SEQ ID Nos. 1-455 and 466 and combinations thereof. Alternatively, the protein is encoded for by a DNA sequence having at least about 85%, more preferably 90%, still more preferably 93%, even more preferably 95%, still more preferably 97%, even more preferably 98%, still more preferably 99% and most preferably 100% sequence homology with a sequence selected from the group consisting of SEQ ID No. 456 and SEQ ID. No. 457, or a portion thereof coding for an immunogenic portion of the proteins encoded by the sequences of SEQ ID No. 456 and SEQ ID No. 457. More preferably, the protein is selected from the group consisting of extracellular and outer membrane Lawsonia proteins. Still more preferably, the protein is selected from the group consisting of SEQ ID Nos. 355, 11, 378, 50, 35, 231, 4, 328, 313, 27, 172, 275, 387, 134, 201, 256, 2, 12, 404, 388, 327, 306, 415, 343, 373, 214, 330, 316, 428, 190, 129, 320, 381, 9, 292, 158, 270, 336, 423, 211, 178, 430, 77, 186, 264, 140, 193, 192, 208, 183, 108, 109, 87, 253, 379, 243, 364, 51, 99, 419, 278, 295, 349, 219, 127, 389, 254, 263, 294, 315, 257, 443, 403, 76, 75, 73, 344, 74, 238, 6, 329, 296, 413, 194, 143, 146, 333, 438, 188, 261, 237, 336, 291, 151, 26, 139, 333, 444, 308, 131, 284, 340, 466, and combinations thereof. Still more preferably, the protein is selected from the group consisting of SEQ ID Nos. 344, 466, and combinations thereof. More preferably, the immunological protein or combination of proteins reacts with convalescent swine serum in a Western blot. In another embodiment of the present invention, the immunological protein has a similar function and/or generates a similar immune response as a protein coded by either SEQ ID No. 456 or SEQ ID No. 457 or a protein selected from the group consisting of SEQ ID Nos. 1-455 and 466, or a portion thereof, or a nucleotide sequence coding for an immunogenic portion of the proteins encoded by the sequences of SEQ ID No. 456 and SEQ ID No. 457, or portion thereof.

In another embodiment of the present invention, the DNA coding for an immunological protein derived from Lawsonia intracellularis is delivered to a desired host using a DNA vaccine. Preferably, the protein is selected from the group consisting of Lawsonia proteins. More preferably, the immunological protein is coded for by a DNA sequence coding for a protein having at least 85%, more preferably 90%, still more preferably 93%, even more preferably 95%, still more preferably 97%, even more preferably 98%, still more preferably 99% and most preferably 100% sequence homology with a sequence selected from the group consisting of SEQ ID Nos. 1-455 and 466, homologs thereof, immunogenic portions thereof, homologs of said immunogenic portions, proteins that have similar functions and induce similar immune responses as any one of SEQ ID Nos. 1-455 and 466, and combinations thereof. Alternatively, the protein is encoded for by a DNA sequence having at least about 85%, more preferably 90%, still more preferably 93%, even more preferably 95%, still more preferably 97%, even more preferably 98%, still more preferably 99% and most preferably 100% sequence homology with a sequence selected from the group consisting of SEQ ID No. 456 and SEQ ID No. 457, or a portion thereof coding for an immunogenic portion of the proteins encoded by the sequences of SEQ ID No. 456 and SEQ ID No. 457. More preferably, the protein is selected from the group consisting of extracellular and outer membrane Lawsonia proteins. Still more preferably, the protein is selected from the group consisting of SEQ ID Nos. 355, 11, 378, 50, 35, 231, 4, 328, 313, 27, 172, 275, 387, 134, 201, 256, 2, 12, 404, 388, 327, 306, 415, 343, 373, 214, 330, 316, 428, 190, 129, 320, 381, 9, 292, 158, 270, 336, 423, 211, 178, 430, 77, 186, 264, 140, 193, 192, 208, 183, 108, 109, 87, 253, 379, 243, 364, 51, 99, 419, 278, 295, 349, 219, 127, 389, 254, 263, 294, 315, 257, 443, 403, 76, 75, 73, 344, 74, 238, 6, 329, 296, 413, 194, 143, 146, 333, 438, 188, 261, 237, 336, 291, 151, 26, 139, 333, 444, 308, 131, 284, 340, 466, and combinations thereof. Still more preferably, the protein is selected from the group consisting of SEQ ID Nos. 344, 466, and combinations thereof. More preferably, the immunological protein or combination of proteins reacts with convalescent swine serum in a Western blot. In another embodiment of the present invention, the immunological protein has a similar function and/or generates a similar immune response as a protein coded by either SEQ ID No. 456 or SEQ ID No. 457 or a protein selected from the group consisting of SEQ ID Nos. 1-455 and 466.

In still another embodiment of the present invention, the DNA coding for an immunological protein derived from Lawsonia intracellularis could be expressed in a prokaryotic or eukaryotic system, then purified and delivered to the desired host. Preferably, the protein is selected from the group consisting of Lawsonia proteins. More preferably, the immunological protein is coded for by a DNA sequence coding for a protein having at least 85%, more preferably 90%, still more preferably 93%, even more preferably 95%, still more preferably 97%, even more preferably 98%, still more preferably 99% and most preferably 100% sequence homology with a sequence selected from the group consisting of SEQ ID Nos. 1-455 and 466 and combinations thereof. Alternatively, the protein is encoded for by a DNA sequence having at least about 85%, more preferably 90%, still more preferably 93%, even more preferably 95%, still more preferably 97%, even more preferably 98%, still more preferably 99% and most preferably 100% sequence homology with a sequence selected from the group consisting of SEQ ID No. 456 and SEQ ID No. 457. More preferably, the protein is selected from the group consisting of extracellular and outer membrane Lawsonia proteins. Still more preferably, the protein is selected from the group consisting of SEQ ID Nos. 355, 11, 378, 50, 35, 231, 4, 328, 313, 27, 172, 275, 387, 134, 201, 256, 2, 12, 404, 388, 327, 306, 415, 343, 373, 214, 330, 316, 428, 190, 129, 320, 381, 9, 292, 158, 270, 336, 423, 211, 178, 430, 77, 186, 264, 140, 193, 192, 208, 183, 108, 109, 87, 253, 379, 243, 364, 51, 99, 419, 278, 295, 349, 219, 127, 389, 254, 263, 294, 315, 257, 443, 403, 76, 75, 73, 344, 74, 238, 6, 329, 296, 413, 194, 143, 146, 333, 438, 188, 261, 237, 336, 291, 151, 26, 139, 333, 444, 308, 131, 284, 340, 466, and combinations thereof. Still more preferably, the protein is selected from the group consisting of SEQ ID Nos. 344, 466, and combinations thereof. More preferably, the immunological protein or combination of proteins reacts with convalescent swine serum in a Western blot. In another embodiment of the present invention, the immunological protein has a similar function and/or generates a similar immune response as a protein coded by either SEQ ID No. 456 or SEQ ID No. 457 or a protein selected from the group consisting of SEQ ID Nos. 1-455 and 466.

Additionally, other vaccination methods known in the art such as IM injection, biodegradable microspheres, or inhalation, among others, may be used for the delivery of an immunological protein in accordance with the present invention.

Thus, the present invention relates to an immunological or immunogenic protein, preferably of Lawsonia intracellularis that is selected from the group of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2)
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, 9, or most preferably 8 contiguous amino acids included in the sequences of SEQ ID No: 1-455, SEQ ID. No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

The immunogenic proteins described herein can be obtained from Lawsonia intracellularis by isolation and/or purification, or can be obtained from in vitro recombinant expression of the nucleic acid(s), coding for the immunogen(s) or portions or epitopes thereof. Methods for the isolation and/or purification of known proteins are well known to a person skilled in the art. Moreover, several methods are known in the art to recombinantly express a protein of a known sequence.

A further aspect of the present invention relates to a DNA molecule that includes a nucleotide sequence, that encodes for at least one of the immunological proteins described above. Preferably, that DNA molecule includes a nucleotide sequence which encodes for at least one immunological protein selected from the group consisting of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2); and/or
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, 9, or most preferably 8 contiguous amino acids included in the sequences of SEQ ID No: 1-455, SEQ ID No: 456, or the amino acid sequence encoded by SEQ ID No: 457 or SEQ ID No: 466.

In still another embodiment of the present invention, the DNA coding for an immunological protein derived from Lawsonia intracellularis is expressed in a prokaryotic or eukaryotic system, then purified and delivered to the desired host. Preferably, the protein is selected from the group consisting of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 0.97% sequence homology, still even more preferably at least about 98% sequence homology, and even more preferably at least about 99% sequence homology to the polypeptide of 1);
3) any immunogenic portion of the polypeptides of 1) and/or 2); and/or
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466.

According to a further aspect, the present invention also relates to a vector comprising any of the DNA molecules described herein. Preferably, that DNA molecule includes a nucleotide sequence which encodes for at least one immunological protein selected from the group consisting of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2); and/or 4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466.

Methods for making and/or using vectors (or recombinants) for expression can be by or analogous to the methods disclosed in: U.S. Pat. Nos. 4,603,112, 4,769,330, 5,174,993, 5,505,941, 5,338,683, 5,494,807, 4,722,848, 5,942,235, 5,364,773, 5,762,938, 5,770,212, 5,942,235, 382,425, PCT publications WO 94/16716, WO 96/39491, WO 95/30018, Paoletti, “Applications of pox virus vectors to vaccination: An update,” PNAS USA 93: 11349-11353, October 1996, Moss, “Genetically engineered poxviruses for recombinant gene expression, vaccination, and safety,” PNAS USA 93: 11341-11348, October 1996, Smith et al., U.S. Pat. No. 4,745,051, (recombinant baculovirus), Richardson, C. D. (Editor), Methods in Molecular Biology 39, “Baculovirus Expression Protocols” (1995 Humana Press Inc.), Smith et al., “Production of Huma Beta Interferon in Insect Cells Infected with a Baculovirus Expression Vector”, Molecular and Cellular Biology, December, 1983, Vol. 3, No. 12, p. 2156-2165; Pennock et al., “Strong and Regulated Expression of Escherichia coli B-Galactosidase in Infect Cells with a Baculovirus vector,” Molecular and Cellular Biology March 1984, Vol. 4, No. 3, p. 399-406; EPA0 370 573, U.S. application No. 920,197, filed Oct. 16, 1986, EP Patent publication No. 265785, U.S. Pat. No. 4,769,331 (recombinant herpesvirus), Roizman, “The function of herpes simplex virus genes: A primer for genetic engineering of novel vectors,” PNAS USA 93:11307-11312, October 1996, Andreansky et al., “The application of genetically engineered herpes simplex viruses to the treatment of experimental brain tumors,” PNAS USA 93: 11313-11318, October 1996, Robertson et al. “Epstein-Barr virus vectors for gene delivery to B lymphocytes”, PNAS USA 93: 11334-11340, October 1996, Frolov et al., “Alphavirus-based expression vectors: Strategies and applications,” PNAS USA 93: 11371-11377, October 1996, Kitson et al., J. Virol. 65,3068-3075, 1991; U.S. Pat. Nos. 5,591,439, 5,552,143, WO 98/00166, allowed U.S. application Ser. Nos. 08/675,556, and 08/675,566 both filed Jul. 3, 1996 (recombinant adenovirus), Grunhaus et al., 1992, “Adenovirus as cloning vectors,” Seminars in Virology (Vol. 3) p. 237-52, 1993, Ballay et al. EMBO Journal, vol. 4, p. 3861-65, Graham, Tibtech 8, 85-87, April, 1990, Prevec et al., J. Gen Virol. 70, 42434, PCT WO 91/11525, Felgner et al. (1994), J. Biol. Chem. 269, 2550-2561, Science, 259:1745-49, 1993 and McClements et al., “Immunization with DNA vaccines encoding glycoprotein D or glycoprotein B, alone or in combination, induces protective immunity in animal models of herpes simplex virus-2 disease”, PNAS USA 93: 11414-11420, October 1996, and U.S. Pat. Nos. 5,591,639, 5,589,466, and 5,580,859, as well as WO 90/11092, WO93/19183, WO94/21797, WO95/11307, WO95/20660, Tang et al., Nature and Furth et al. Analytical Biochemistry, relating to DNA expression vectors, inter alia. See also WO 98/33510; Ju et al., Diabetologia, 41: 736-739, 1998 (lentiviral expression system); Sanford et al., U.S. Pat. No. 4,945,050; Fischbach et al. (Intracel), WO 90/01543; Robinson et al., seminars in Immunology vol. 9, pp. 271-283 (1997), (DNA vector systems); Szoka et al., U.S. patent No. (method of inserting DNA into living cells); McCormick et al., U.S. Pat. No. 5,677,178 (use of cytopathic viruses); and U.S. Pat. No. 5,928,913 (vectors for gene delivery), as well as other documents cited herein. A viral vector, for instance, selected from pig herpes viruses, such as Aujeszky's disease virus, porcine adenovirus, poxviruses, especially vaccinia virus, avipox virus, canarypox virus, and swinepox virus, as well as DNA vectors (DNA plasmids) are advantageously employed in the practice of the invention.

According to a further aspect the present invention relates to an immunological composition, preferably a vaccine composition, effective for lessening the severity of clinical symptoms associated with a Lawsonia intracellularis infection. Preferably, that immunological composition comprises an immunological protein, a DNA molecule coding for an immunological protein, and/or a vector including a DNA coding for an immunological protein as disclosed herein. Preferably, said immunological protein is:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2)
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

The immunogenic and vaccine compositions of the present invention can include diluents, isotonic agents, stabilizers, and/or adjuvants, preferably selected from those which are disclosed herein.

Thus, according to a further aspect, the present invention relates to a immunological composition, that comprises an immunological protein, an DNA molecule coding for an immunological protein, and/or an vector including a DNA coding for an immunological protein described herein and a diluents, isotonic agents, stabilizers, or adjuvants. Preferably, said immunological protein is:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2)
4) the immunogenic portion of 3), comprising at least 300, 29.0, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

Preferably said diluent, isotonic agent, stabilizer, or adjuvant is anyone of those described above.

In another embodiment of the present invention, there is provided a method for the prevention or treatment of an animal against Lawsonia intracellularis infections by inoculating said animal with an immunological protein derived from Lawsonia intracellularis. Preferably, the protein or immunological composition is anyone of those described above. Preferably, said immunological protein is:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2)
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

In another embodiment of the present invention, the animal is vaccinated by inoculating it with a vaccine prepared by inserting DNA coding for an immunological protein derived from Lawsonia intracellularis into a vector and administering the vector through any conventional means. One preferred method of administration is oral. Preferably, the vector is a bacteria. More preferably, the vector is salmonella. Preferably, the DNA codes for a protein selected from the group consisting of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2)
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

More preferably, the immunological protein or combination of proteins coded by said DNA molecule reacts with convalescent swine serum in a Western blot.

In another embodiment of the present invention, the DNA molecule coding for an immunological protein derived from Lawsonia intracellularis is delivered to a desired host using a DNA vaccine. Preferably, the DNA molecule expresses the immunological protein, when it has entered a host cell. Preferably, the immunological protein encoded by the DNA molecule is selected from the group consisting of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2); and/or
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466.

More preferably, the immunological protein or combination of proteins reacts with convalescent swine serum in a Western blot.

The vaccine compositions of the present invention, as disclosed herein, can further include one or more other immunomodulatory agents such as, e.g., interleukins, interferons, or other cytokines. The vaccine compositions can also include Gentamicin and Merthiolate. While the amounts and concentrations of adjuvants and additives useful in the context of the present invention can readily be determined by the skilled artisan, the present invention contemplates compositions comprising from about 50 ug to about 2000 ug of adjuvant and preferably about 250 ug/ml dose of the vaccine composition. In another preferred embodiment, the present invention contemplates vaccine compositions comprising from about 1 ug/ml to about 60 ug/ml of antibiotics and/or immunomodulatory agents, and more preferably less than about 30 ug/ml of antibiotics and/or immunomodulatory agents.

According to a further embodiment, vaccine compositions in accordance with the present invention can first be dehydrated. If the composition is first lyophilized or dehydrated by other methods, then, prior to vaccination, said composition is rehydrated in aqueous (e.g. saline, PBS (phosphate buffered saline)) or non-aqueous solutions (e.g. oil emulsion (mineral oil, or vegetable/metabolizable oil based/single or double emulsion based), aluminum-based, carbomer based adjuvant).

Vaccine or immunogenic compositions according to the invention may be administered intramuscularly, intranasally, orally, intradermally, intratracheally, or intravaginally. Preferably, the composition is administered intramuscularly, orally, or intranasally. In an animal body, it can prove advantageous to apply the compositions as described above via an intravenous injection or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months and in different dosages.

Another aspect of the present invention provides a diagnostic/detection assay utilizing proteins in accordance with the invention. Preferably, that diagnostic/detection assay is specific for the detection of antibodies in a sample which specifically reacts with antigen of Lawsonia intracellularis. Preferably, that diagnostic/detection assay is specific for the detection of antibodies in a sample, wherein those antibodies are generated in cause of a Lawsonia intracellularis infection. Preferably, the protein is selected from the group consisting of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2);
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

Such proteins could be used in an ELISA-based test. Such a protein could also be injected into an animal (e.g. a rabbit) to create an antiserum useful for detecting antibody or, antigen. Such assays would be useful in confirming or ruling out Lawsonia infection.

Preferably the detection assay, preferably the ELISA-based test, comprises the steps:
1) contacting a sample comprising antibodies against Lawsonia intracellularis bacteria with an immunogenic protein of Lawsonia as described herein;
2) incubating the mixture of 1) under conditions which allow the immunogenic protein of Lawsonia to bind to the Lawsonia specific antibodies of the sample and to generate a complex of Lawsonia specific antibody and the immunogenic protein; and
3) Detecting the presence of the complex of 2).

Another aspect of the present invention relates to a kit in parts, comprising an protein selected from the group consisting of:
1) a polypeptide comprising a sequence selected from the group consisting of SEQ ID Nos: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2)
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

Preferably that kit in parts is a detection kit for the detection of antibodies in a sample which specifically react with antigen of Lawsonia intracellularis. Preferably, that detection kit is specific for the detection of antibodies in a sample, wherein those antibodies are generated in cause of a Lawsonia intracellularis infection.

Another aspect of the present invention provides an expression system for expressing proteins useful for purposes of the present invention. Those of skill in the art are familiar with such expression systems. A preferred expression system in this regard will utilize E. coli or recombinant baculovirus to express or generate recombinant proteins. Preferably, the E. coli or baculovirus will have nucleic acid sequences inserted therein which encode for proteins, as described above. It is noted that the examples of expression systems are mentioned above in an exemplarily manner.

In another aspect of the present invention, fusion proteins and chimeras are provided. Preferably, the fusion proteins or chimera present or expressed will comprise any one of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2);
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID No: 466; and/or
5) a polypeptide that is encoded by a DNA that codes for a peptide comprising the sequence of SEQ ID No: 1-455 or SEQ ID No: 466.

### BRIEF DESCRIPTION OF TH E DRAWING FIGURES

FIG. 1 is a Coomasie stained Gel picture illustrating the expression of the Omp85-like protein;

FIG. 2 is picture of the IMAC fractions of E. coli (pET HlyA);

FIG. 3 is a gel picture of the HlyA and Omp85-like proteins;

FIGS. 4A-C are Western Blot pictures showing reactivity to the HlyA and Omp85-like proteins of the present invention;

FIG. 5 provides the results of a BLAST search showing the homologous data for the 456 Lawsonia proteins; and

FIG. 6 is a listing of the 456 Lawsonia proteins, with the first 6 proteins being preceded by the protein name and being SEQ ID Nos. 1-6, respectively, and the remaining 450 proteins being preceded by their corresponding SEQ ID No.

### DETAILED DESCRIPTION OF THE PREFERRED EMBODIMENTS

The following examples set forth preferred materials and procedures in accordance with the present invention. It is to be understood, however, that these examples are provided by way of illustration only, and nothing therein should be deemed a limitation upon the overall scope of the invention.

### EXAMPLE 1

This example demonstrates the immunological detection of the Lawsonia intracellularis DK15540 hemolysin A (HlyA) and Omp85 proteins expressed as prokaryotic fusion proteins.

Materials and Methods

Transforming E. coli Strains

To begin, McCoy cell DNA was removed from a Lawsonia intracellularis (“Lawsonia”) cell pellet. This was done by first propagating the DK15540 strain of Lawsonia in a McCoy cell suspension culture. The Lawsonia infected McCoy cells were then pelleted by centrifugation at 10,000 rpm for 30 minutes at 4° C. using a JA-17 rotor (Beckman Coulter, Fullerton, Calif.). The supernatant was removed and the pelleted cells were then disrupted by repeated passage through a 22G double-hub emulsifying needle using two syringes. The disrupted cell mixture was then mixed with 35 mL of a Percoll/NaCl solution. The resulting solution was then centrifuged at 14,000 rpm for 45 minutes at 4° C. After centrifugation, the upper layer of debris was removed with a pipette and the bacterial band was recovered. This bacterial band was then centrifuged at 14,000 rpm for 15 minutes. The resulting Lawsonia pellet was then washed three times by resuspending the pellet in 35 mL of PBS. The resulting suspension was then centrifuged at 14,000 rpm for 15 minutes. The supernatant was discarded and the pellet was resuspended in 3 mL of PBS. Next, 30 μL of 1M MgSO₄ and 30 μL of DNase A was added to the suspension. The resulting mixture was then incubated at 37° C. for 2 hours. The mixture was then diluted to 35 mL with Percoll/NaCl and centrifuged as above (14,000 rpm for 15 minutes at 4° C.). The resulting pellet was washed three times in PBS and then stored overnight at 4° C.

To extract genomic DNA from the Lawsonia cell pellet, the pellet was resuspended in 3.5 mL of buffer B1 from the Qiagen Genomic DNA Kit (Qiagen, Valencia, Calif.) after the overnight storage. Next, 10 μL RNase A (5 μg/μL), 80 μL of lysozyme solution (100 mg/ml) and 100 μL Proteinase K (20 mg/ml). The resulting mixture was incubated at 37° C. for 1 hour and 1.2 mL of Buffer B2 from the Qiagen Genomic DNA kit was added to it. The resulting solution was then gently mixed by inversion. Following the mixing, the solution was then incubated at 50° C. for 30 minutes. While the solution was being incubated, a genomic-tip 500G (from the Qiagen Genomic DNA Kit) was equilibrated with 10 mL of QBT buffer. After incubation, the resulting solution was vortexed for 10 seconds at maximum speed (14,000 rpm) and applied to the pre-equilibrated tip. After the entire solution had entered the tip, it was washed twice with 30 mL of Buffer QC, and the DNA was eluted with 15 mL of Buffer QF. To the eluted DNA was added 10.5 mL of isopropanol, and the tubes were then mixed by gentle inversion. The resulting mixture was then dispensed into separate 1.5 mL microfuge tubes and centrifuged at 14,000 rpm for 15 minutes. The resulting supernatants were then decanted and the pellets rinsed with 0.5 ml of 70% ethanol. The tubes were centrifuged, the supernatants decanted again, and the pellets briefly dried. 12.5 μL of TE buffer was then added to each tube. The tubes were then incubated overnight at 37° C. with gentle shaking. The solutions were then pooled into a single tube, incubated at 55° C. for 2 hours and then quantified by UV spectroscopy.

Next, PCR was performed on the Lawsonia genes and genomic sequence analysis, including BLAST search data, was then used to identify two genes of interest: Omp85 (SEQ ID No. 456) and HlyA (SEQ ID No. 457). The resulting DNA sequence data was used to determine the potential open reading frames (“ORFs”) for each gene and PCR primers were designed which would correspond to the 5′ and 3′ ends of the desired gene with the additional ligation independent cloning (“LIC”) overhang added to the 5′ end of each respective primer (SEQ ID Nos. 458 GGTATTGAGGGTCGCATGACAAAACGCCTGAATATATT and 459 AGAGGAGAGTTAGAGCCTTATTAGAAGAATTGCCCCA for the LIMOP85 primers for pET-32Xa/LIC and SEQ ID Nos. 460 GGTATTGAGGGTCGCATGGCCAAACATAAAGTACGTGC and 461 AGAGGAGAGTTAGAGCCTTATTAACGTTTTTTCAAGTAAA, respectively, for the Hemolysin primers in pET-32Xa.LIC vector). For each of these primers, the underlined portion represents the primer specific sequences required for the LIC process. These sequences are also provided herein as SEQ ID Nos. 462, 463, 464, and 465, respectively. PCR was then carried out using the Lawsonia DK15540 genomic DNA as a template.

For the HlyA PCR cycle, the PCR reaction was heated to 95° C. for 5 minutes. The reaction then proceeded to 35 cycles of 95° C. for 1 minute, 55° C. for 1 minute, and 72° C. for 1 minute. The PCR cycle was completed following a final cycle of 72° C. for 10 minutes.

For the Omp85 PCR cycle, the PCR reaction was heated to 95° C. for 5 minutes. The reaction then proceeded to 35 cycles of 95° C. for 1 minute, 55° C. for 1 minute, and 72° C. for 1.83 minutes. The PCR cycle was completed following a final cycle of 72° C. for 10 minutes.

For both PCR cycles, the reaction mixture comprised 1 μl DNA, 5 μL 10× ExTaq Buffer, 4 μL 2.5 mM dNTP, 1 μL of 10 pm Primer L, 1 L of 10 pm Primer R, 0.5 μL ExTaq, and 38.5 μL of distilled water. The ExTaq Buffer, dNTP, and ExTaq were provided by Takara Bio, Inc. (Japan).

To clone the Lawsonia DK15540 hemolysin and Omp85 ORF for expression analysis, the resulting PCR products were then gel purified using the Qiagen MiniElute Gel Purification kit and mixed with a pET-32Xa LIC plasmid vector and ligated as per the manufacturer's instructions (Novagen, Madison, Wis.). The ligation mixes were used to transform competent cells of NovaBlue® E. coli (Novagen) and plated for ampicillin resistance. The transformed colonies were used to inoculate 3 mL of LB broth and ampicillin and grown overnight at 37° C. A 1.5 mL aliquot of the overnight culture was then harvested by centrifugation at 14,000×g for 2 minutes. The plasmid DNA was then extracted by the Qiagen Mini-Prep plasmid kit. The purified plasmid DNA was then verified by dideoxynucleotide sequencing. The respective plasmids were then transformed into the BL21(DE3) strain of E. coli for prokaryotic fusion protein expression studies.

Expression Analysis

To perform an expression analysis of the transformed E. coli, 10 mL of each of the transformed strains of E. coli (a strain producing hemolysin A and a strain producing Omp85) were incubated overnight in Luria-Bertani (LB) media having 2% glucose w/v and ampicillin (50 μg/ml) at 37° C. with shaking at 225 rpm in a conical tube. The next morning, these two cultures were used to inoculate two separate 10 ml pre-warmed cultures of LB media, glucose 2% and ampicillin (50 μg/ml) at 37° C. with shaking at 225 rpm in a conical tube. The cultures were then grown at 37° C. to an OD600 nm of about 0.8 to about 1.0. This took about 3 to 4 hours. One tube of each strain was then induced with 1 mM isopropyl-beta-D-thiogalactopyranoside (IPTG) for 3 hours at 37° C. The second tube of each strain was left uninduced.

Next, two 1 mL samples of each culture were collected and then pelleted by centrifugation at 20,000×g for one minute. This created two uninduced and two induced samples for each strain. One of each sample (that is, one uninduced and one induced sample of each strain) was then suspended in 400 μL of 1× SDS-Page buffer containing 10 mM 2-ME. The suspensions were then heated to 85° C. for five minutes. Next, the remaining samples (that is one induced and one uninduced sample for each strain) were suspended in 200 μL of 50 mM sodium phosphate, 0.5M NaCl, 5 mM 2-ME, and 1% tergitol. All of the samples were then sonicated for 4 minutes using 0.5 second duty cycles at an amplitude of 75%. The samples that were suspended in the buffer including tergitol were centrifuged for 5 minutes at 20,000×g and the supernatant was then collected while the pellet was discarded.

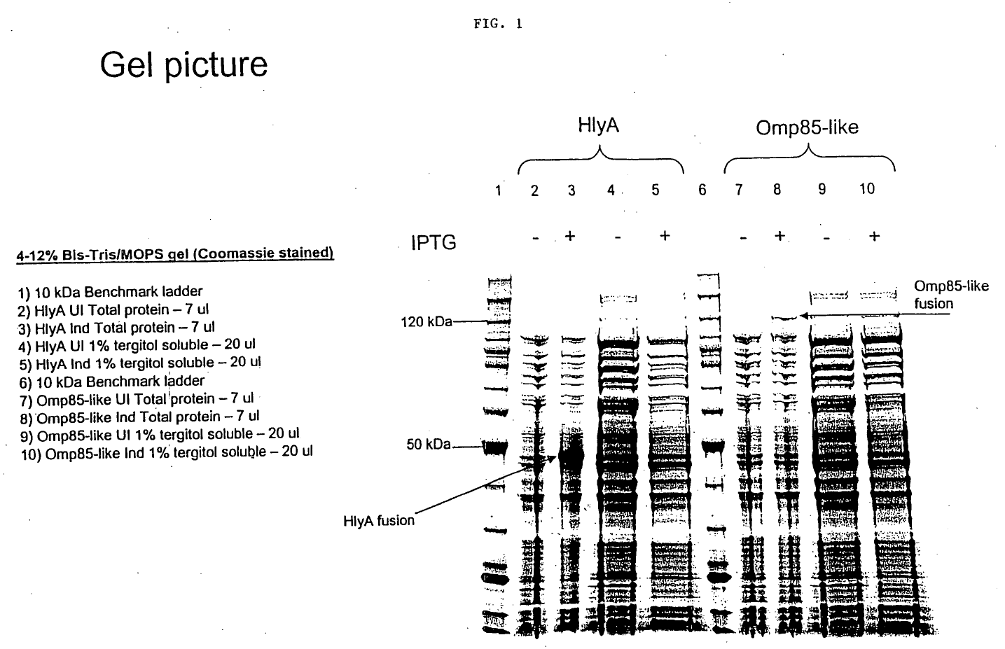

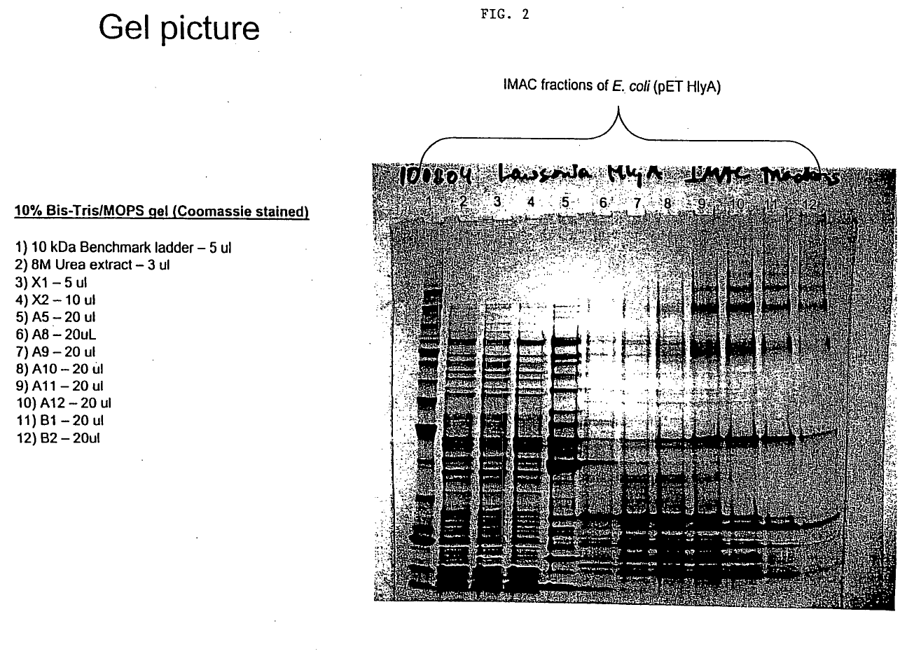

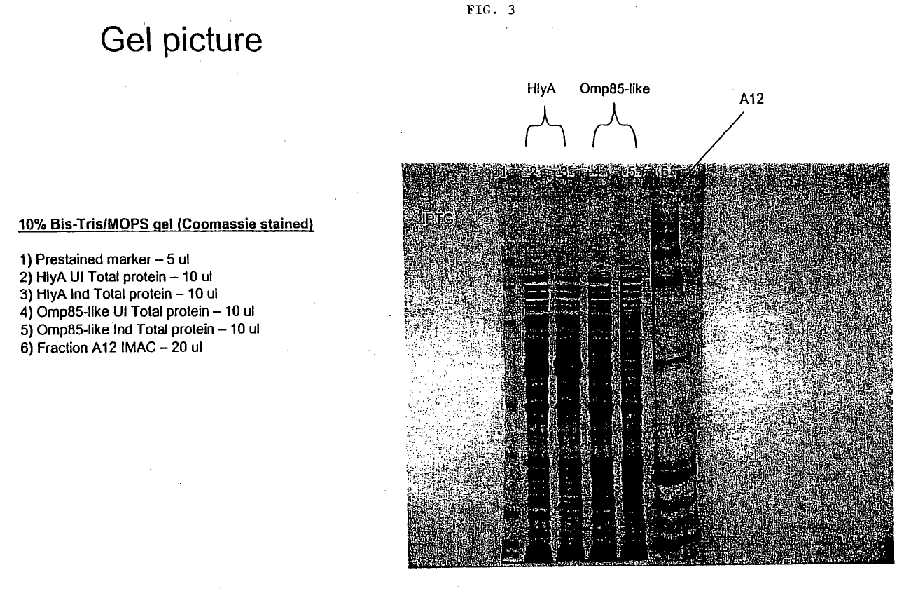

Once prepared in this manner, a western blot analysis of each of the samples was then performed. The resulting gel can be seen as FIG. 1.

As can be seen in the gel, the HlyA protein expression amounted to about 20 to 30% of the total cellular protein. The Omp85-like protein did not express as well, however. Additionally, both proteins were only observed in the total protein induced sample lanes, thereby indicating that these proteins are not soluble in the 1% tergitol buffer.

### EXAMPLE 2

This example demonstrates the purification of hemolysin A and Omp85-like Lawsonia proteins expressed in E. coli cells.

Materials and Methods

10 mL of each of the transformed strains of E. coli were grown overnight in a media of LB, 2% glucose, and 50 μg/mL of Ampicillin. The next morning, the overnight cultures were used to inoculate a IL pre-warmed broth of LB, glucose, and Ampicillin. These cultures were grown at 37° C. for about 3-4 hours until they had reached an OD600 nm of about 0.8 to about 1.0. The cultures were then each induced for 3.5 hours at 37° C. with 0.5 mM IPTG. After induction, the cells were then collected and pelleted by centrifugation at 20,000×g for 20 minutes. The pellet was then suspended in a 33 mL buffer containing 50 mM sodium phosphate, 0.5M sodium chloride, 8M urea, 5 mM 2-ME, and 10 mM imidazole. The resulting suspension was then extracted overnight to disrupt the cells and denature the protein and thereby increase the solubility at 4° C. The extracted samples were then centrifuged for 20 minutes at 20,000×g. The resulting supernatants were then collected and filtered using 0.2 μm syringe filters. 16 mL of each sample was then loaded onto the sample loop and a partial purification was performed using Immobilized-metal affinity chromatography SAC. Following purification, the fractions were collected and a standard SDS-PAGE was performed (4-12% Bis-Tris gel in MOPS buffer). Following the running of the gel, a Coomassie blue stain was performed.

Results and Discussion

The resulting gel can be seen as FIG. 2. As can be seen, expression was not very good in the 1 L culture. However, despite the poor expression yield, there does seem to be some approximately 48 kDA protein in lanes 7-12 from a late gradient eluant peak. Additionally, there appears to be a distinct banding pattern of high molecular weight proteins in lanes 9-12.

### EXAMPLE 3

This example demonstrates the immunological detection of the Omp85-like and Hemolysin A total proteins.

Materials and Methods

The Omp85-like protein, HlyA protein, and IMAC fraction A12 protein were used in three Western blots. The first blot was completed with a Lawsonia ELISA antibody, which was obtained from convalescent pig sera harvested from a 9 week old pig which had previously tested negative for Lawsonia infection by IFAT and ELISA (a “strict control”). The antibody had been diluted to 1:50 in TTBS+2% dry milk. The second blot was completed with swine anti-Lawsonia convalescent serum which had been diluted 1:50 in TTBS+2% dry milk. The third blot was a conjugate-only blot completed using a goat anti-swine HRP which had been diluted 1:1000 in TTBS+2% dry milk (KPL, Inc., Gaithersburg, Md.).

First, the proteins were run through an SDS-PAGE gel (10% Bis/Tris in a MOPS buffer). The proteins were then transferred from the gels to a PVDF membrane at a constant 30V for one hour using a Novex blot module (Invitrogen). The proteins were then blocked for at least one hour in 50 mL TTBS+2% dry milk (w/v). The membranes were then incubated with the antibodies described above. The membranes were then washed 3 times in TTBS (1×TBS+0.05% Tween20), with each wash lasting about 2 minutes. The membranes were then each incubated for an hour with a secondary antibody (goat anti-swine HRP, KPL) which had been diluted to 1:1000 in TTBS+2% dry milk. After incubation, the membranes were washed twice with TTBS, with each wash lasting about 2 minutes. The membranes were then washed once with PBS for about 2 minutes. After the wash, 10 ml Opti-4CN (Bio-Rad, Hercules, Calif.) was added as a substrate. The membranes were then developed for up to 30 minutes, then rinsed with water to stop.

Results and Discussion

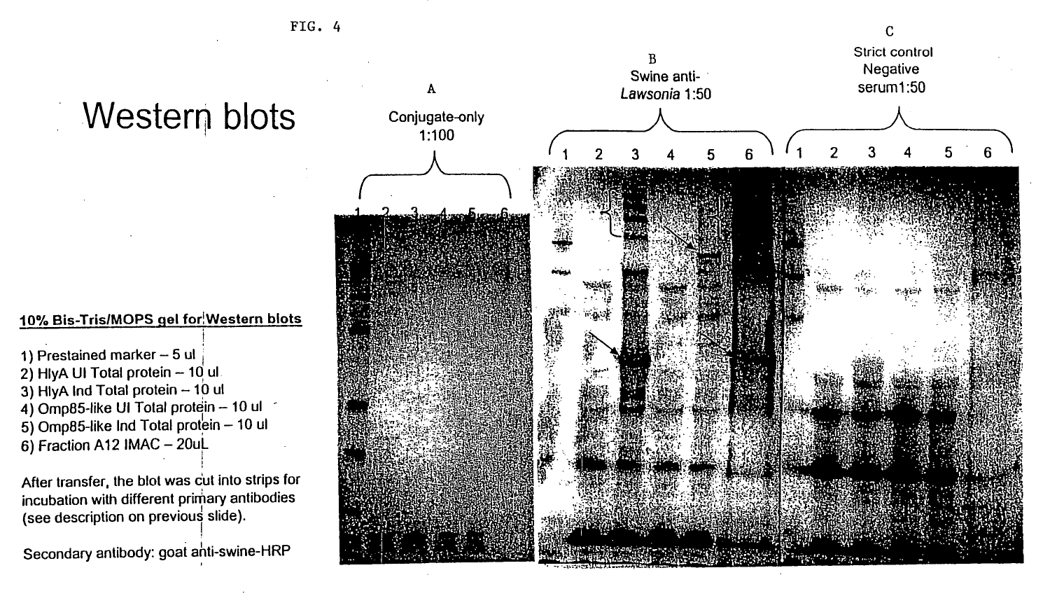

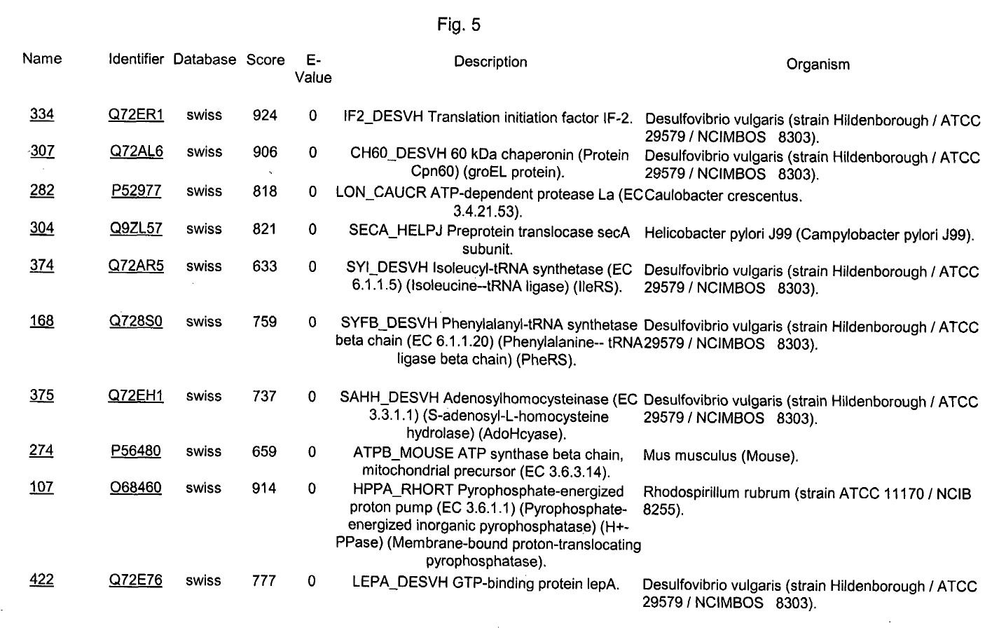

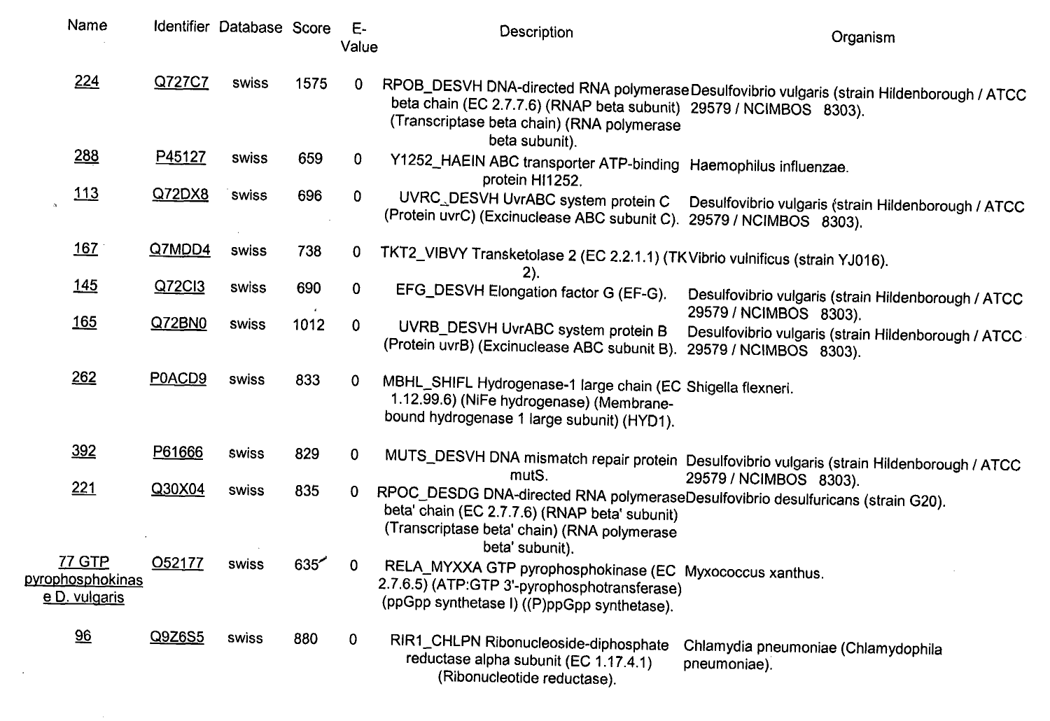

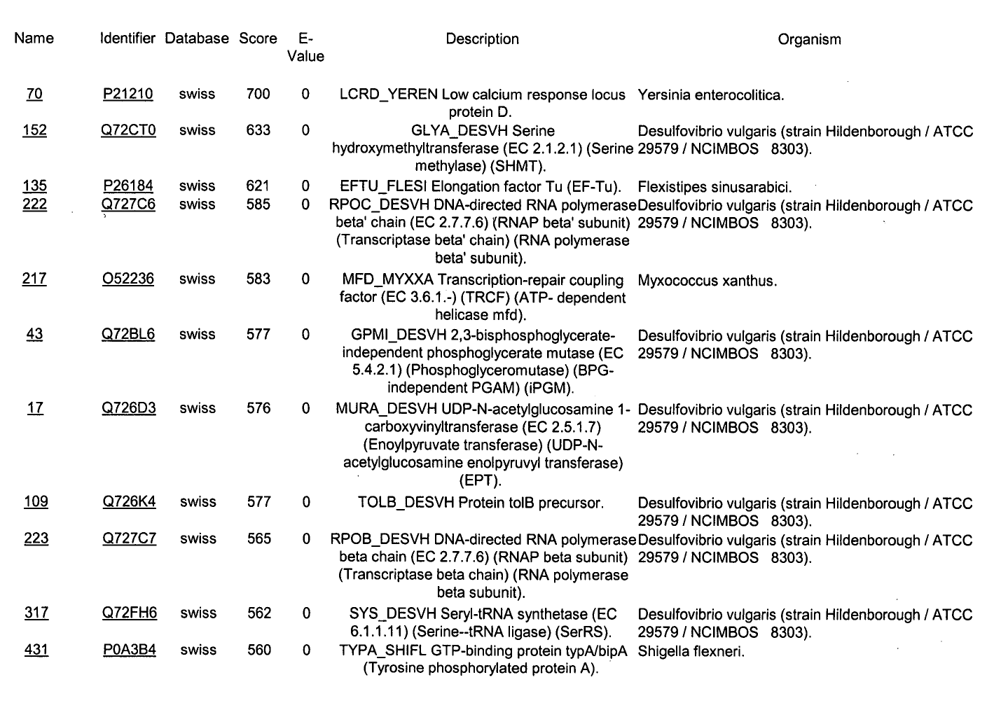

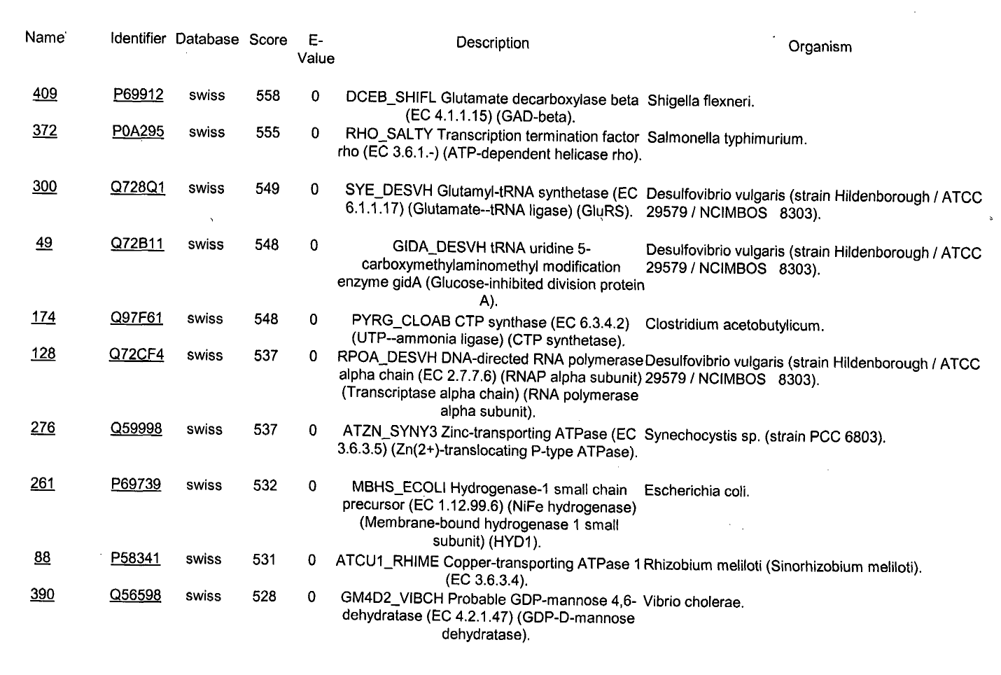

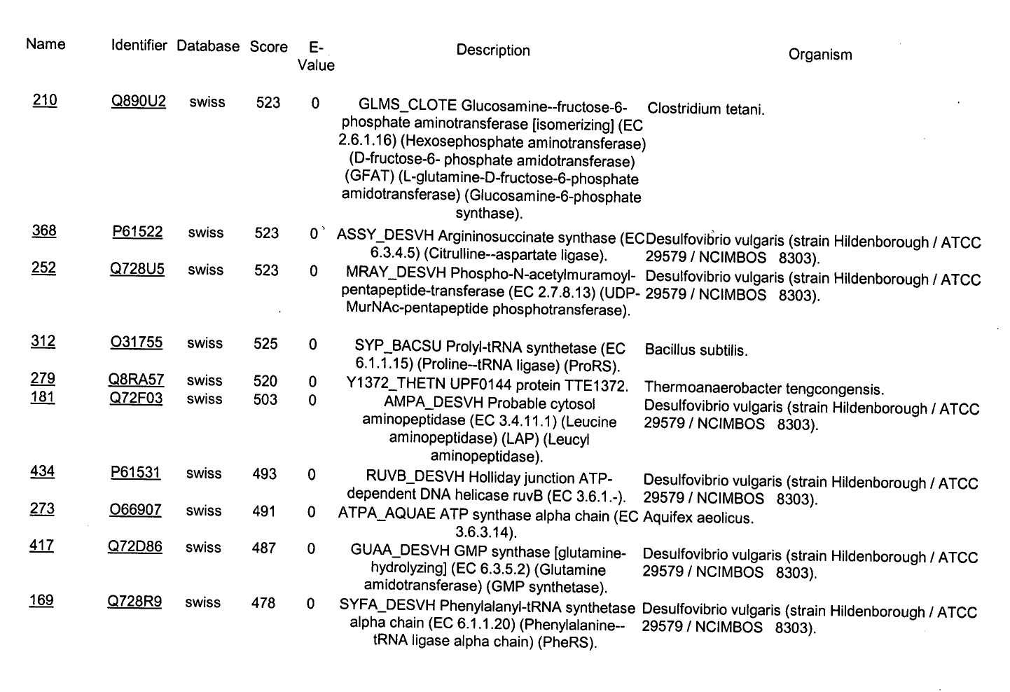

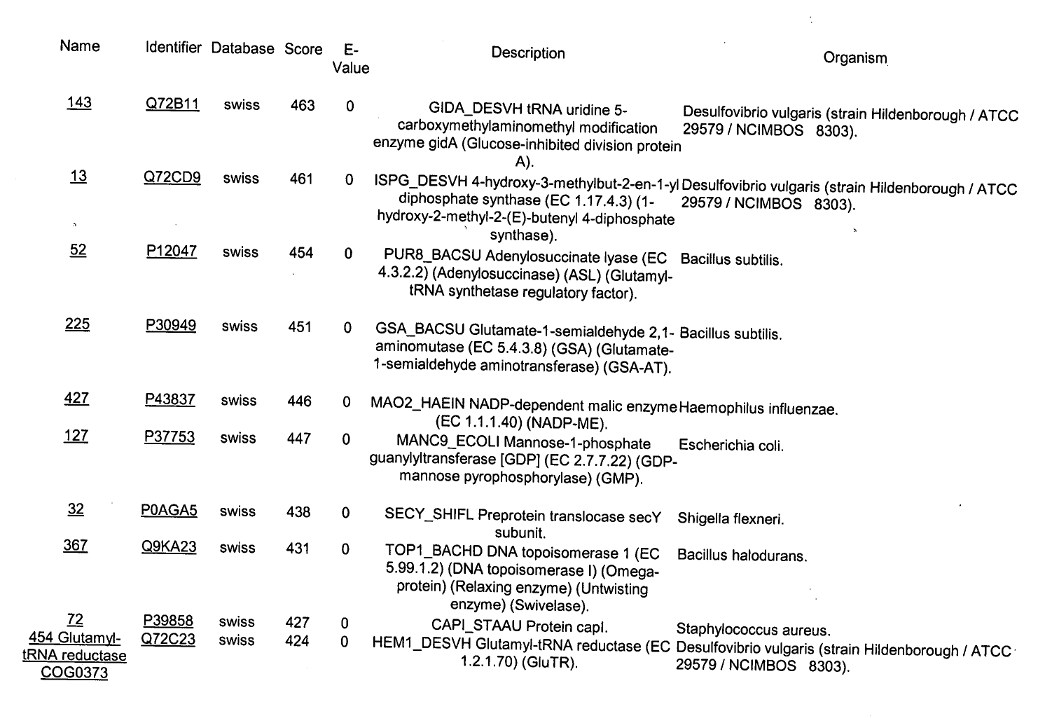

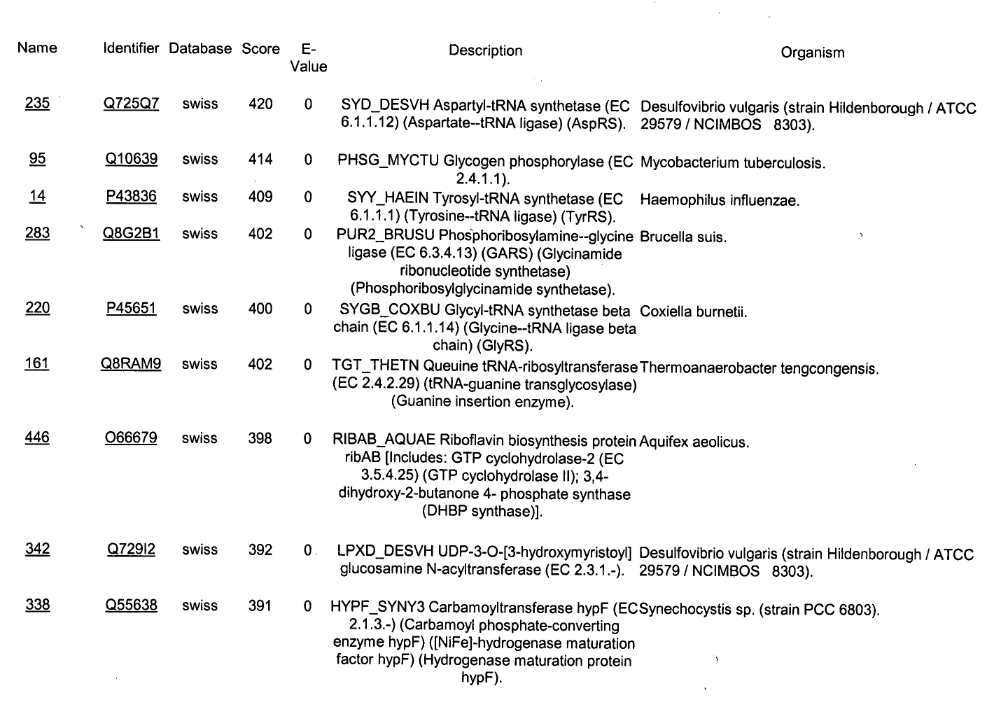

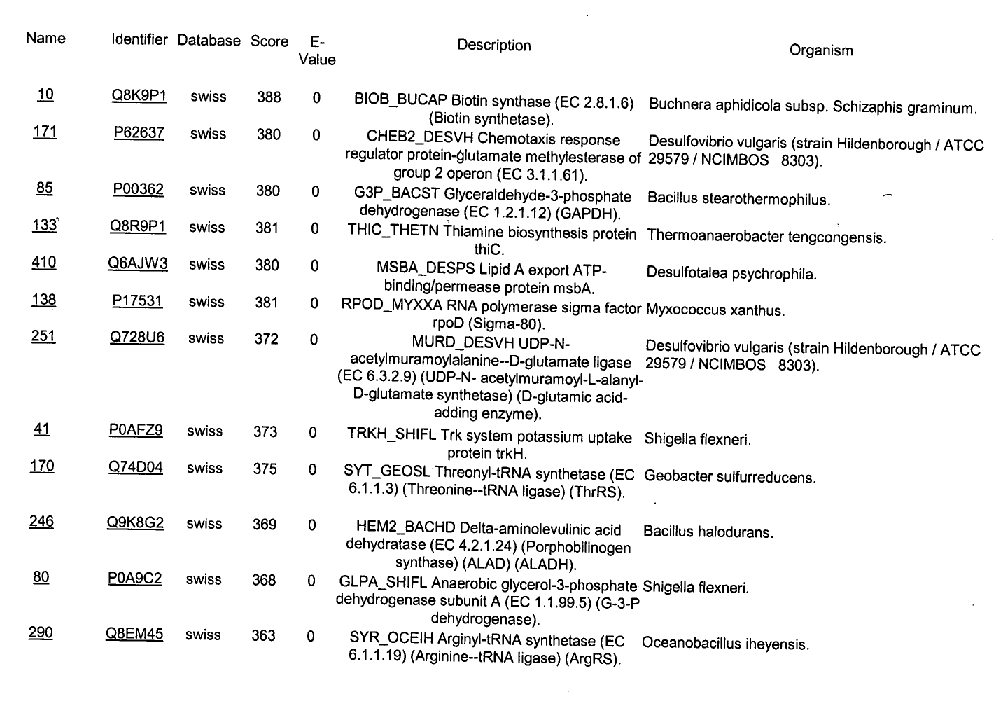

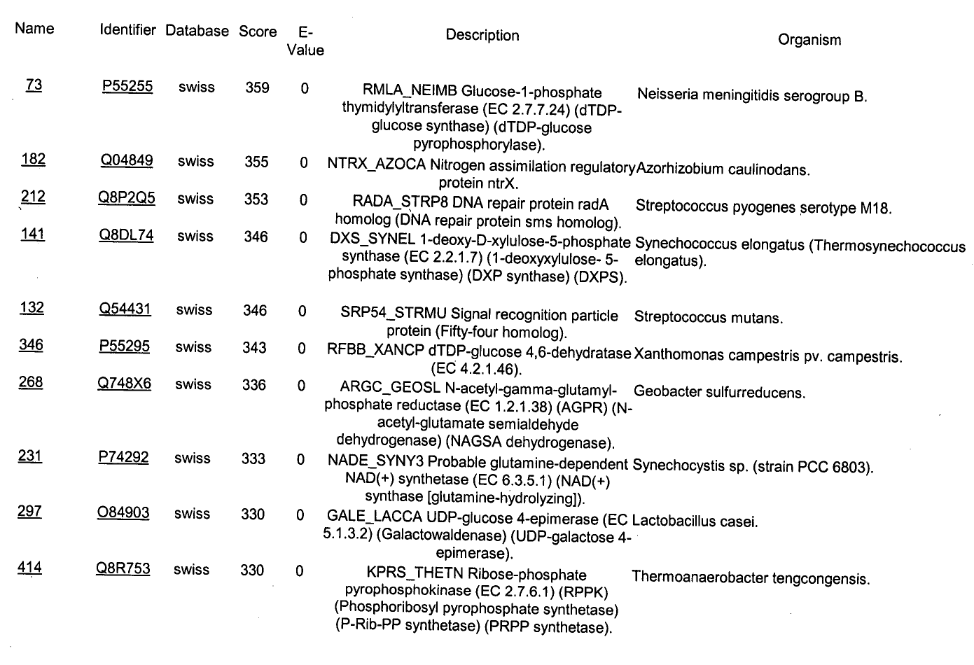

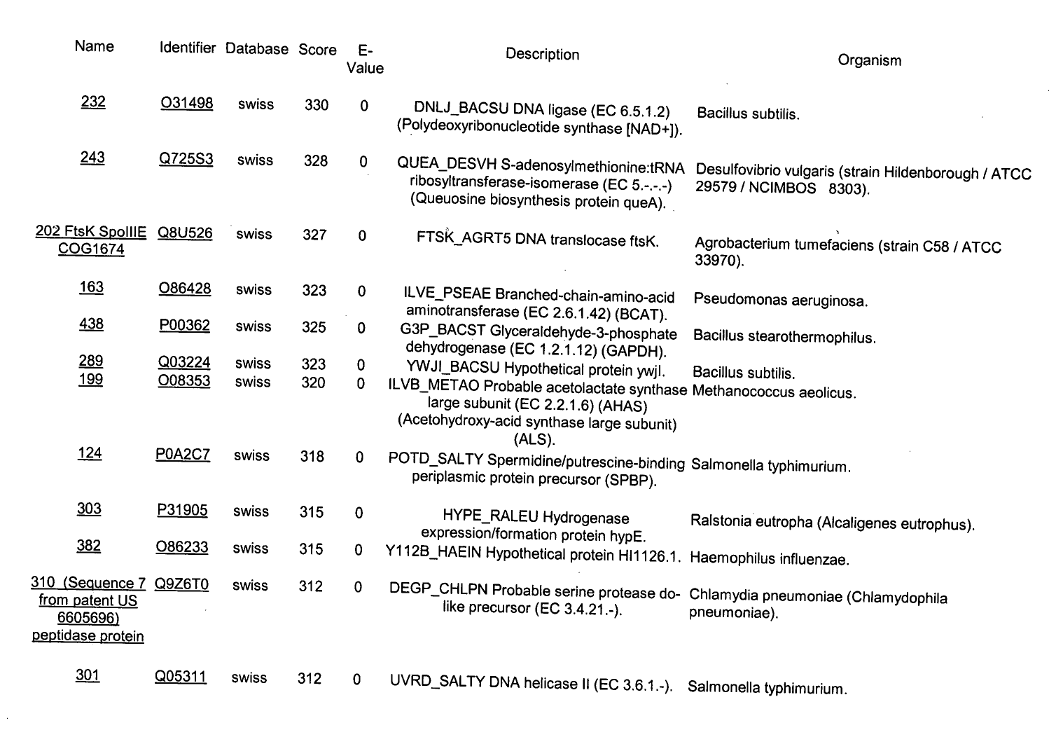

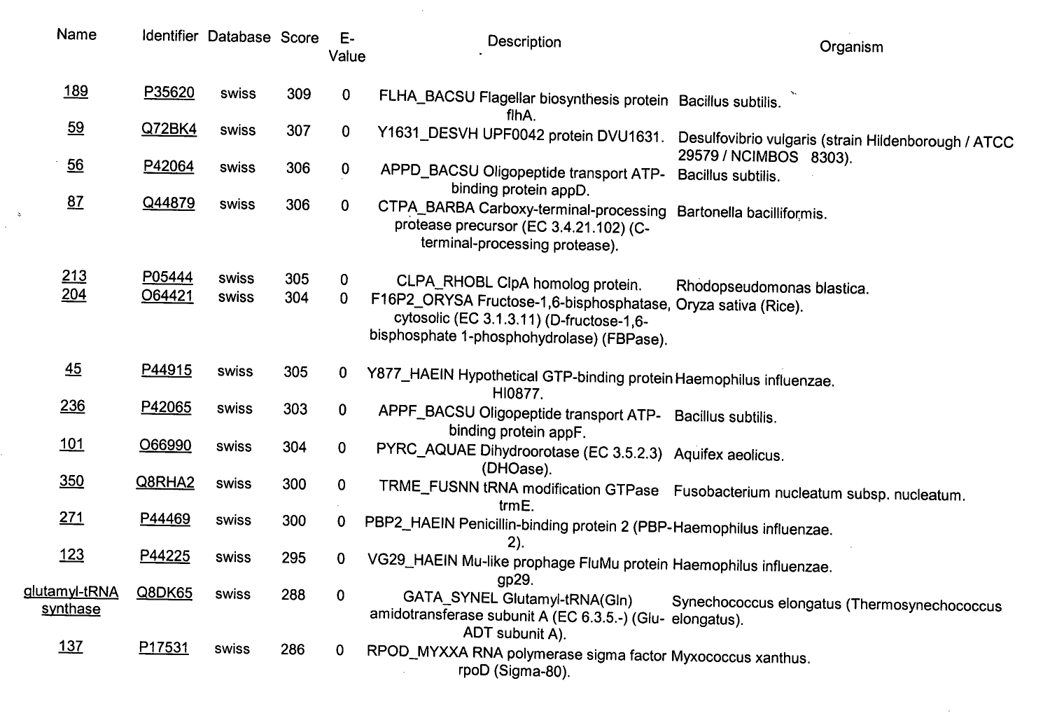

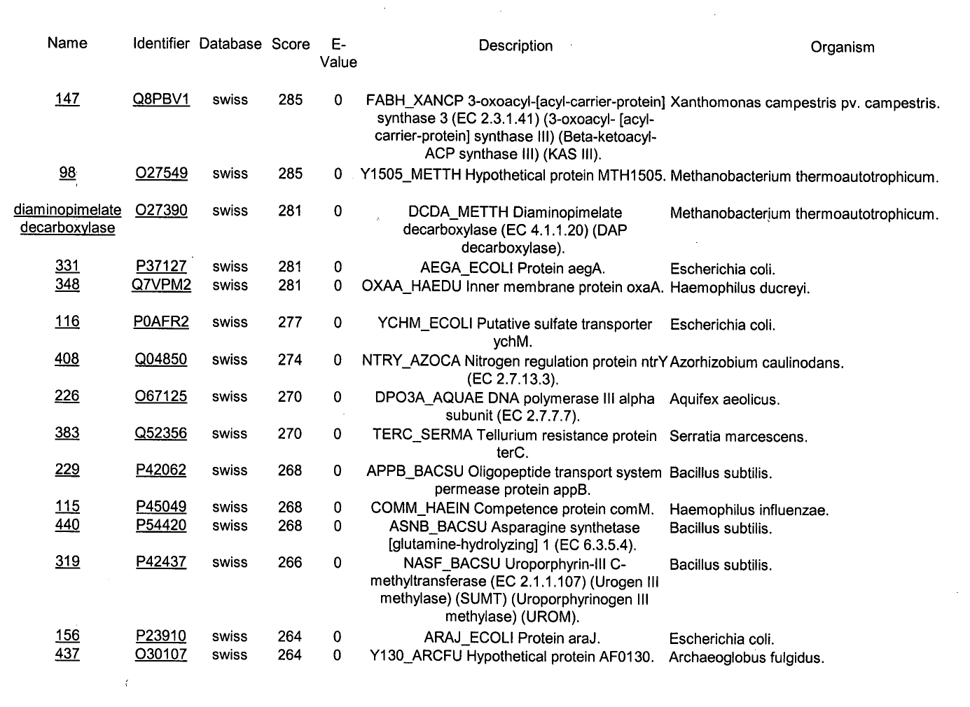

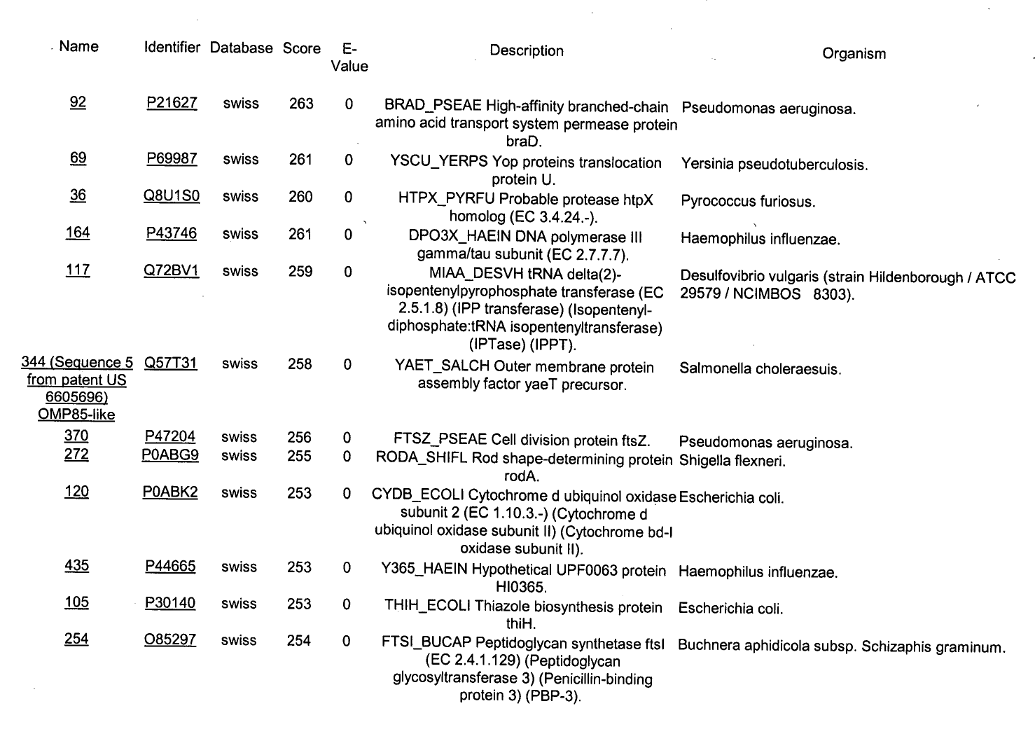

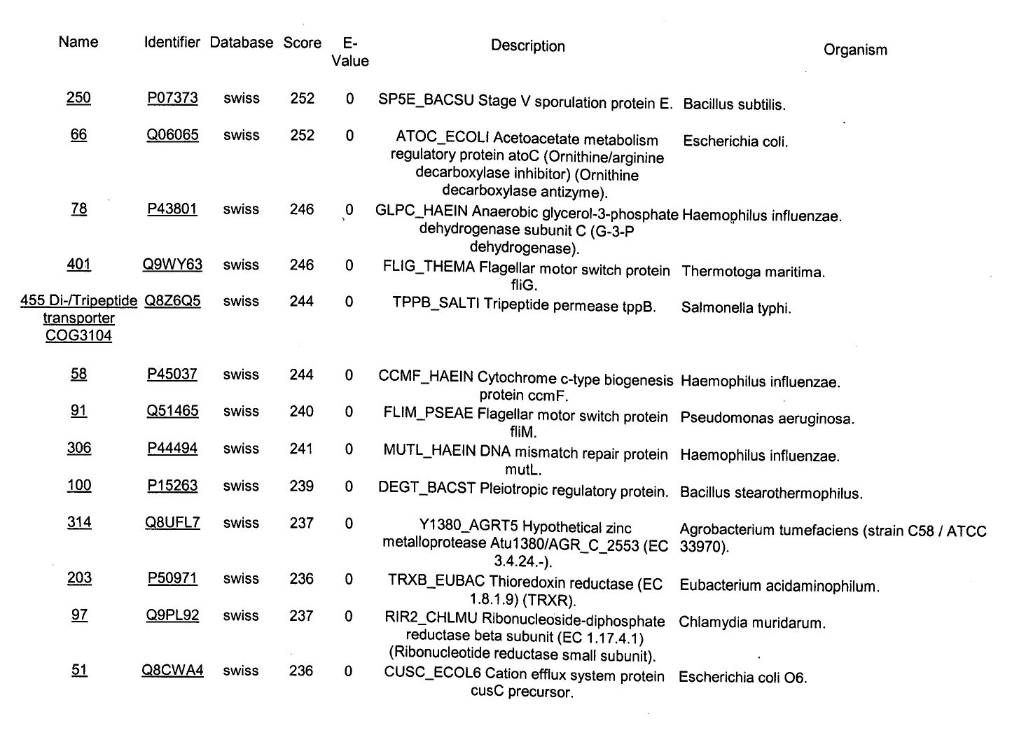

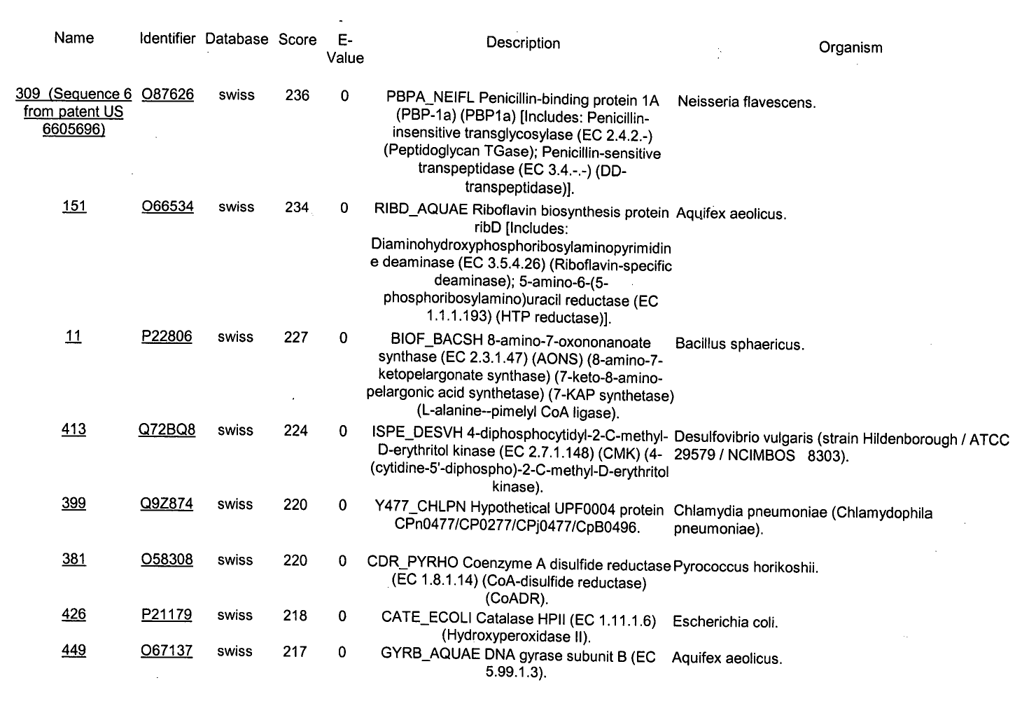

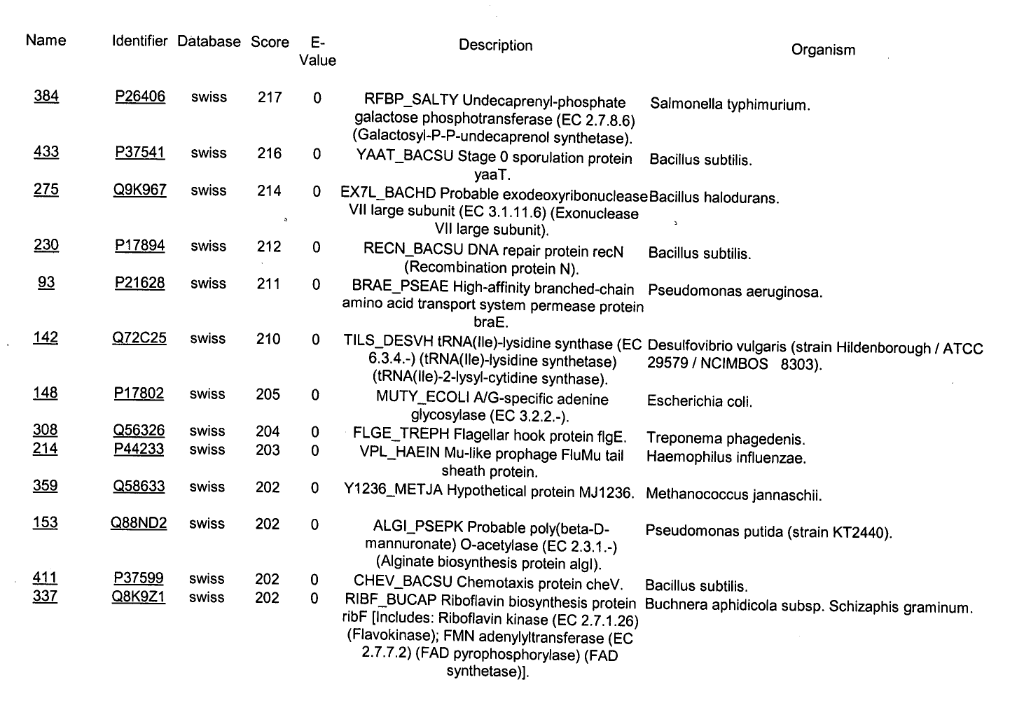

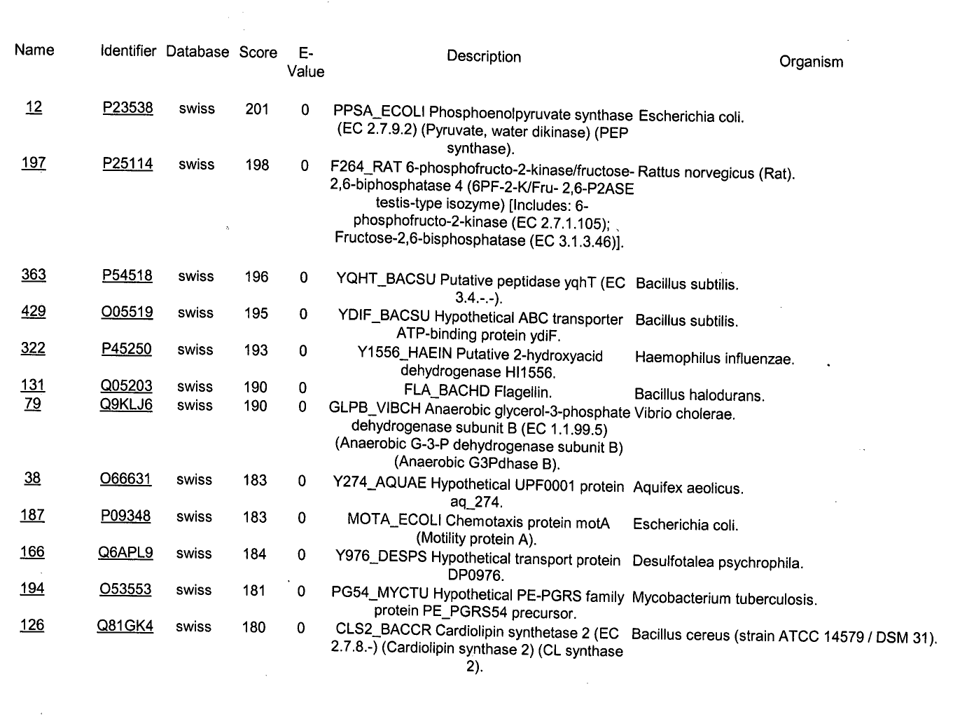

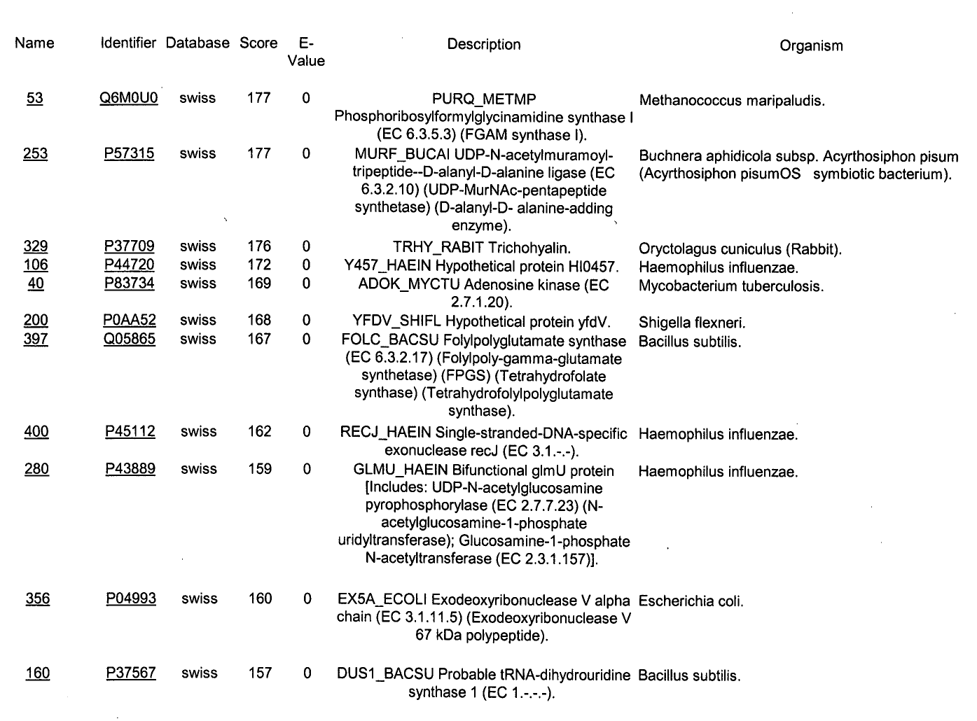

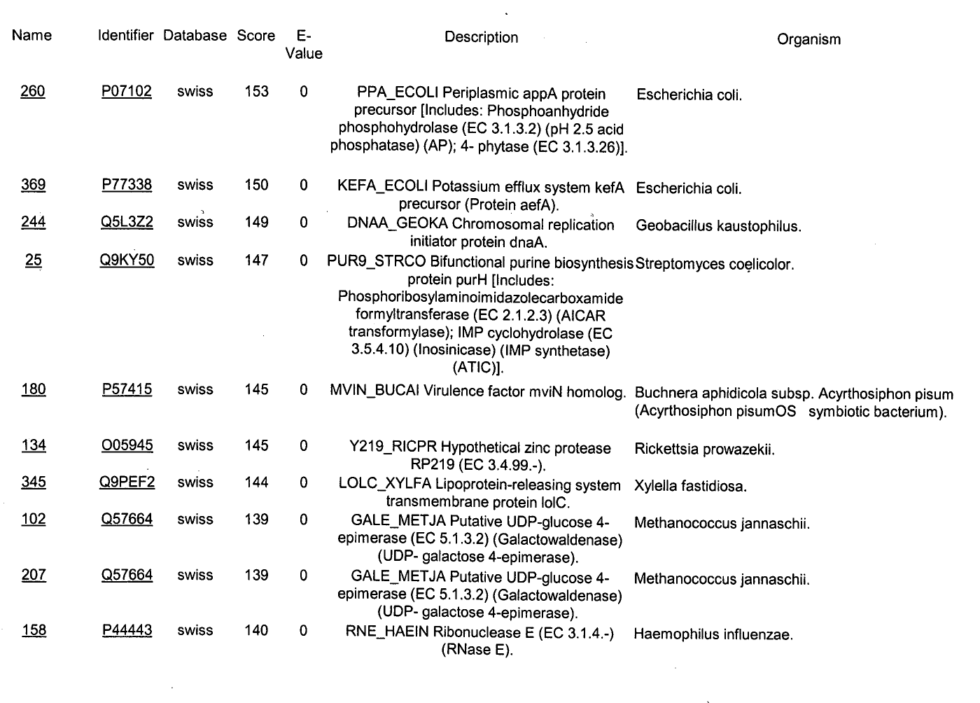

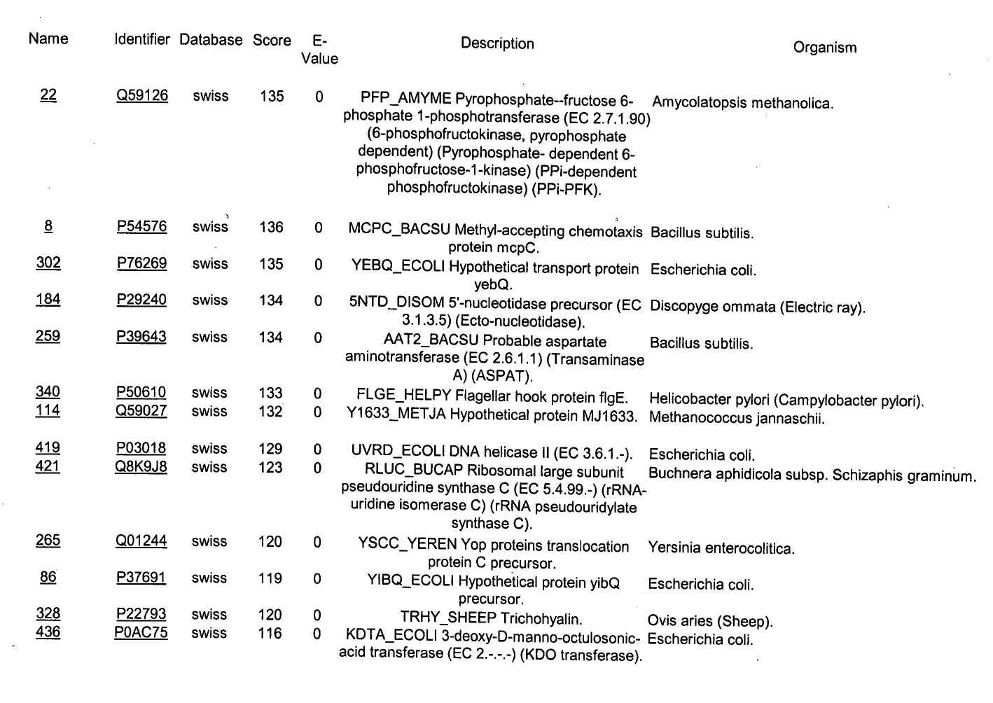

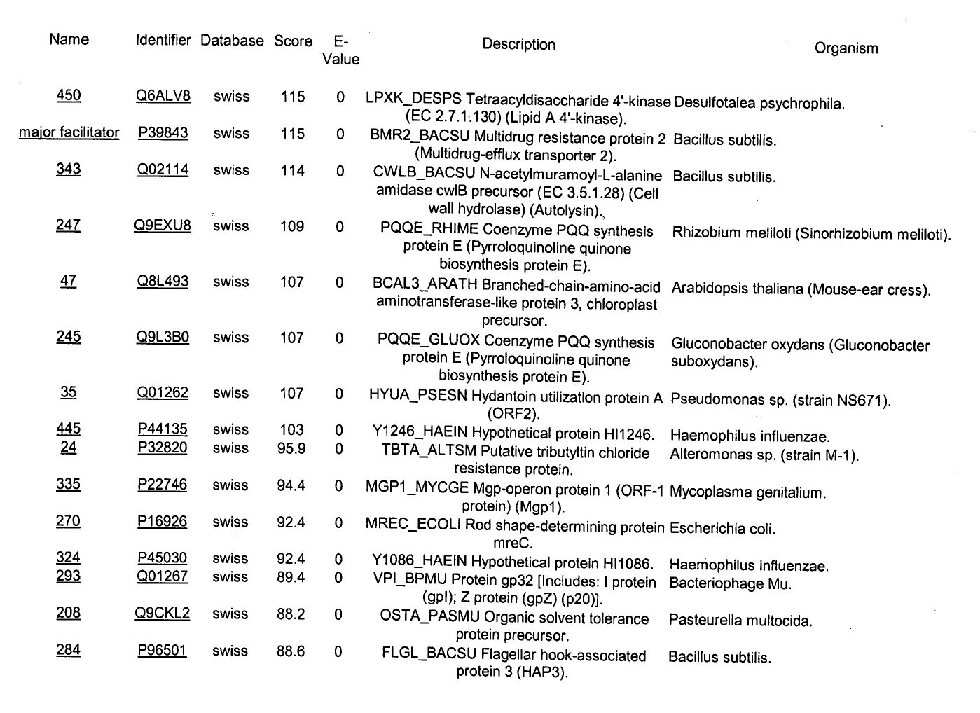

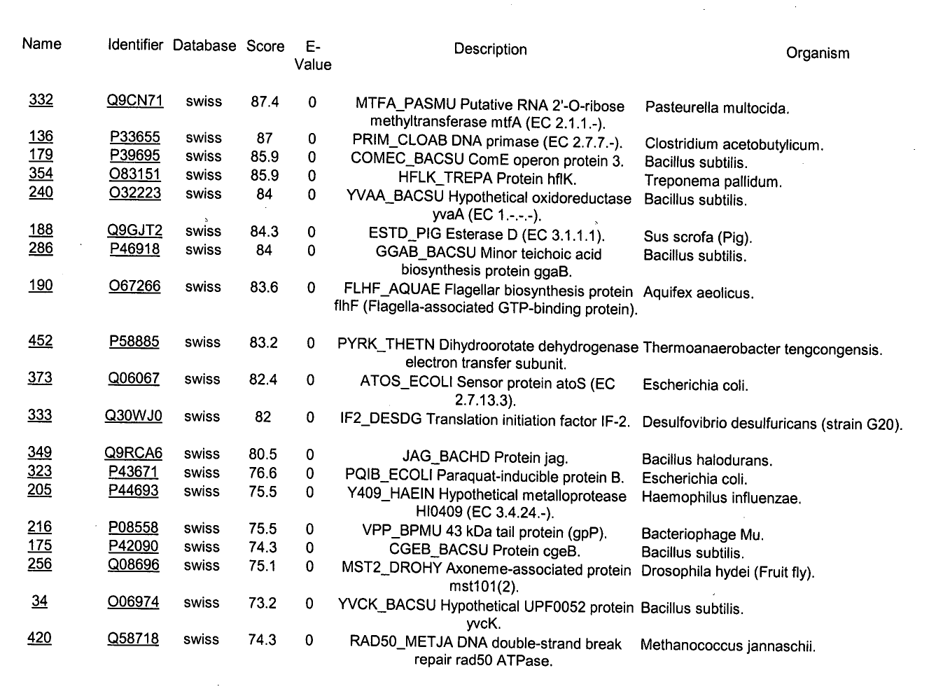

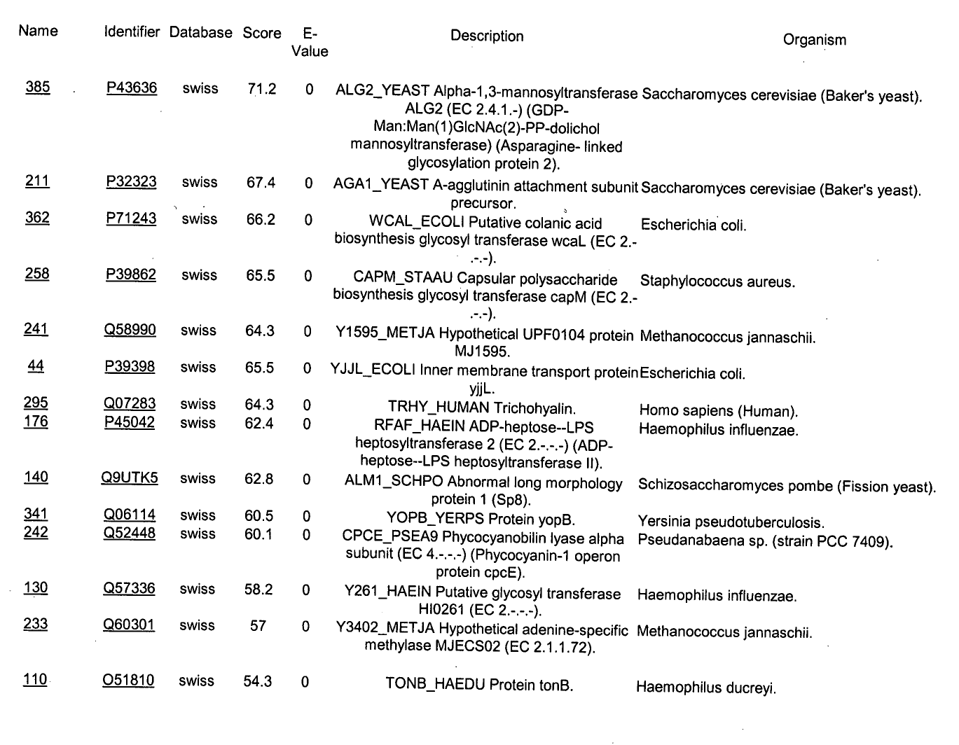

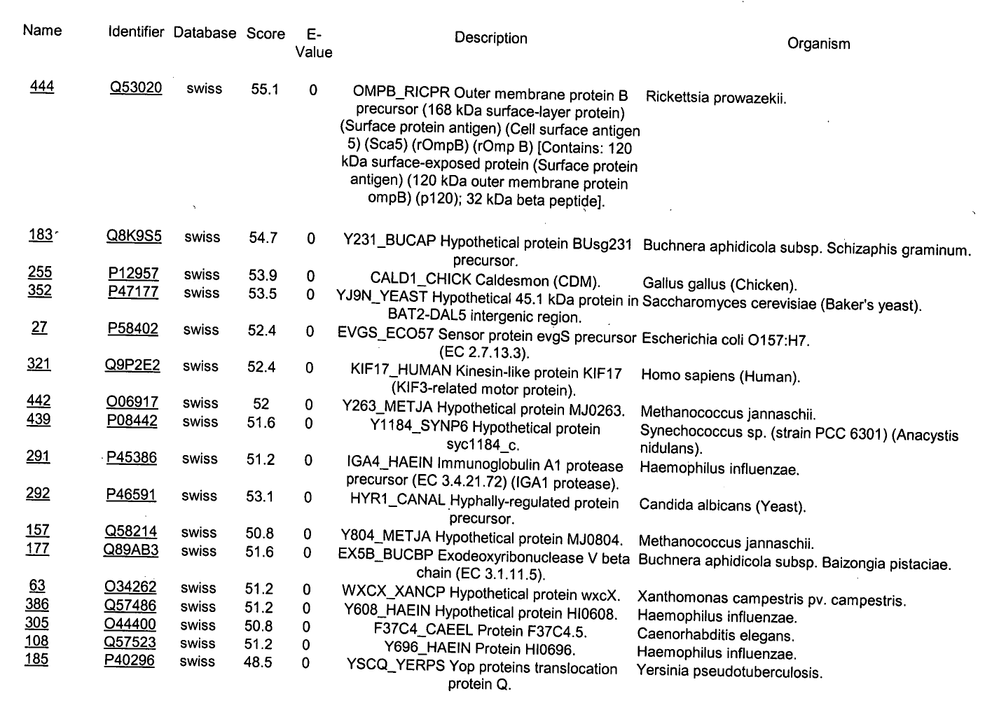

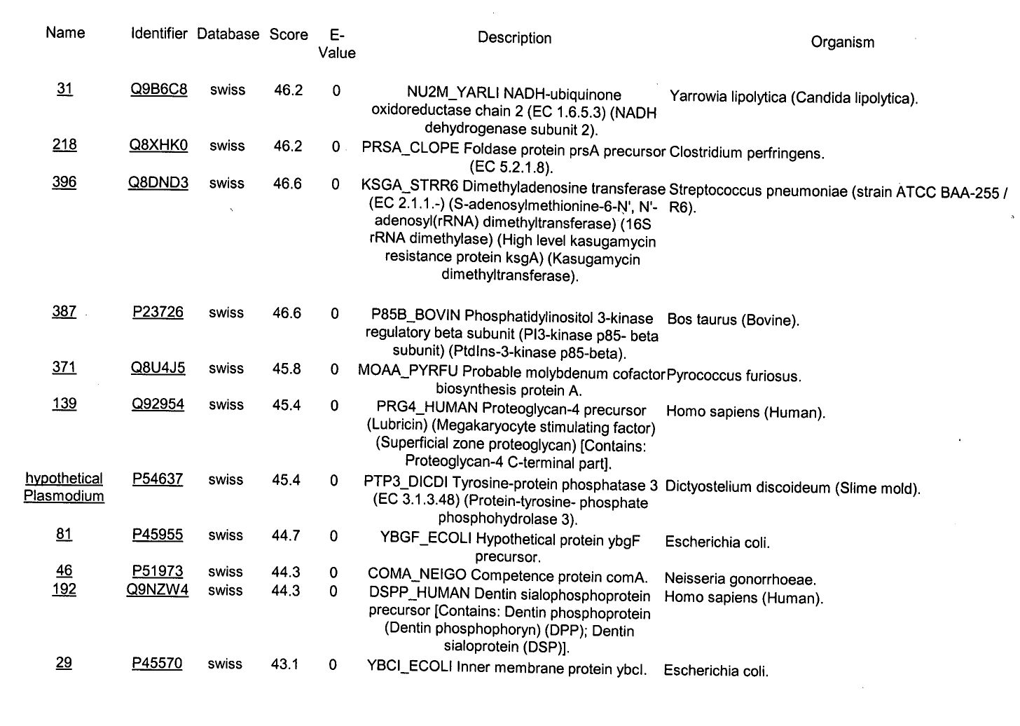

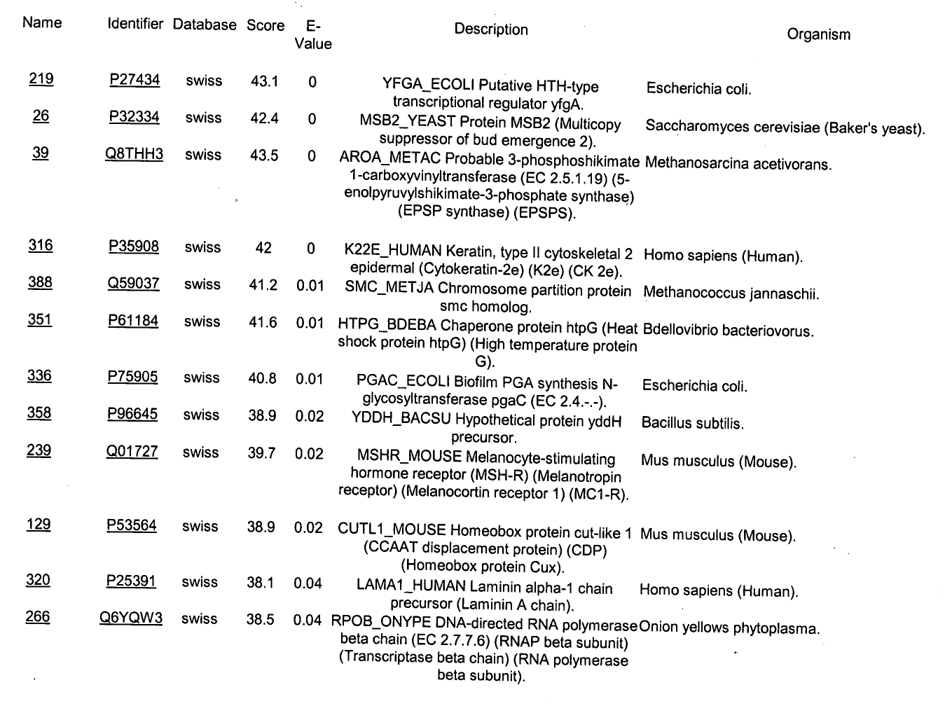

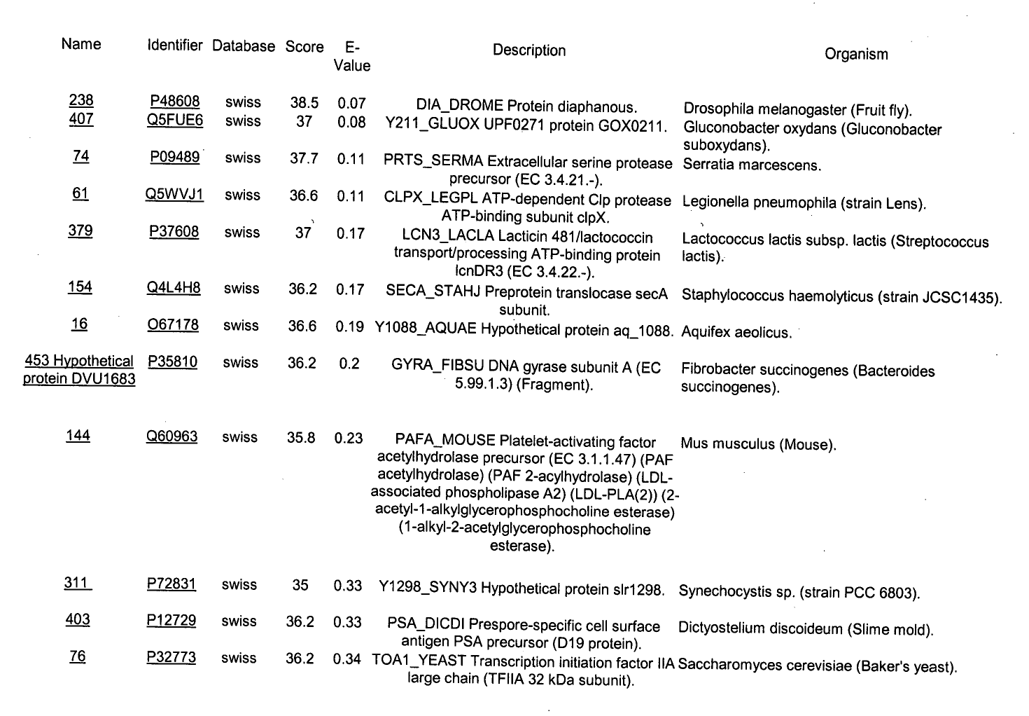

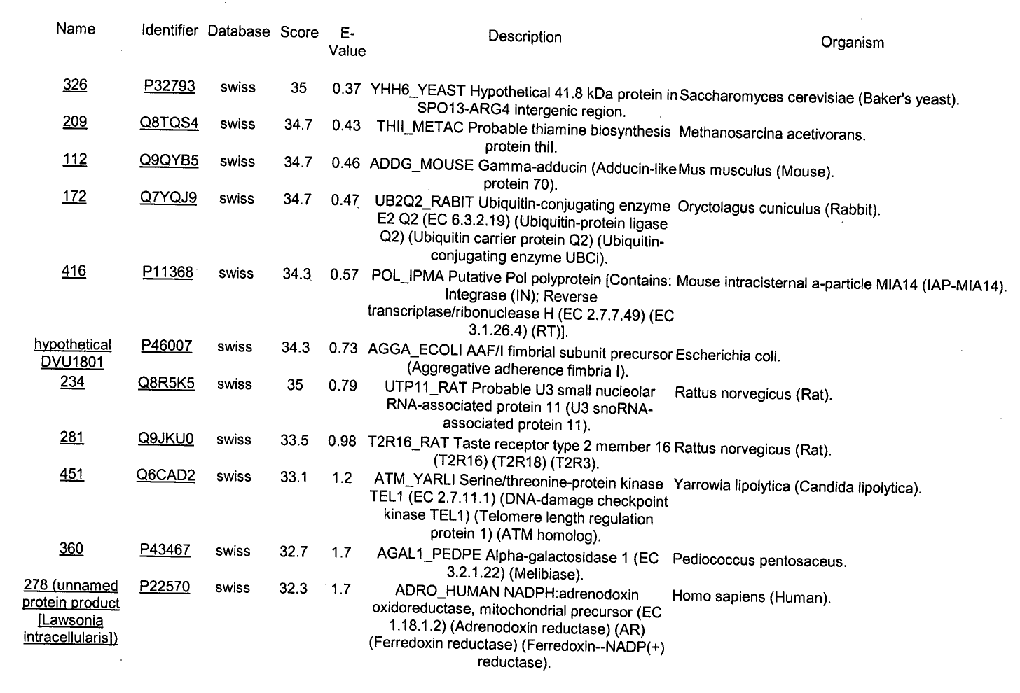

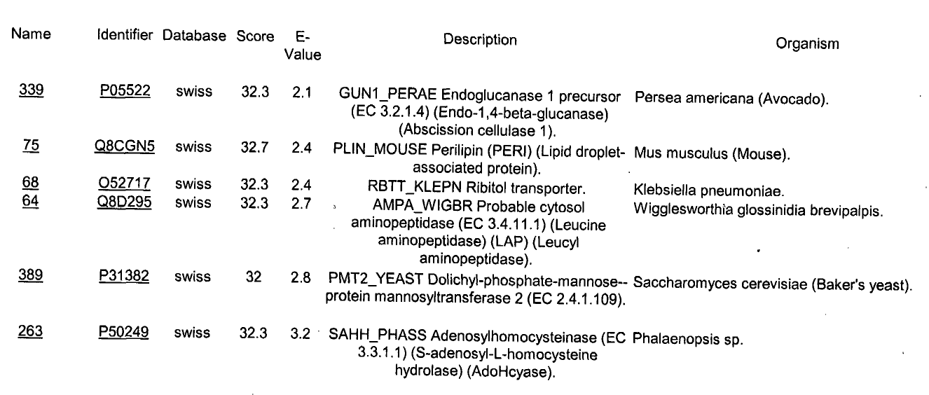

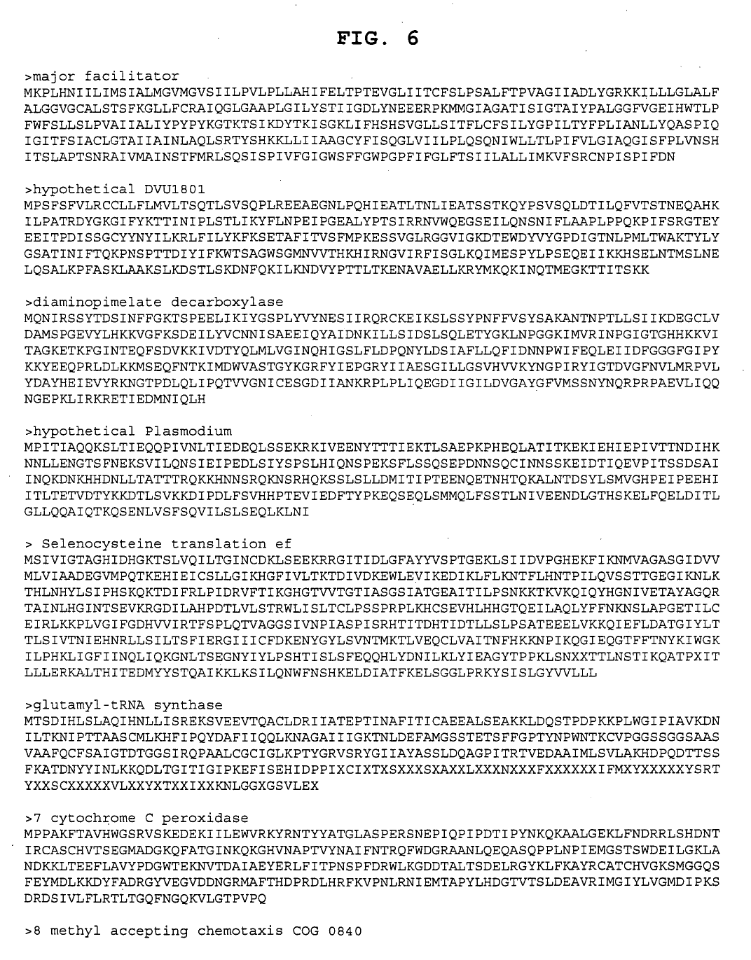

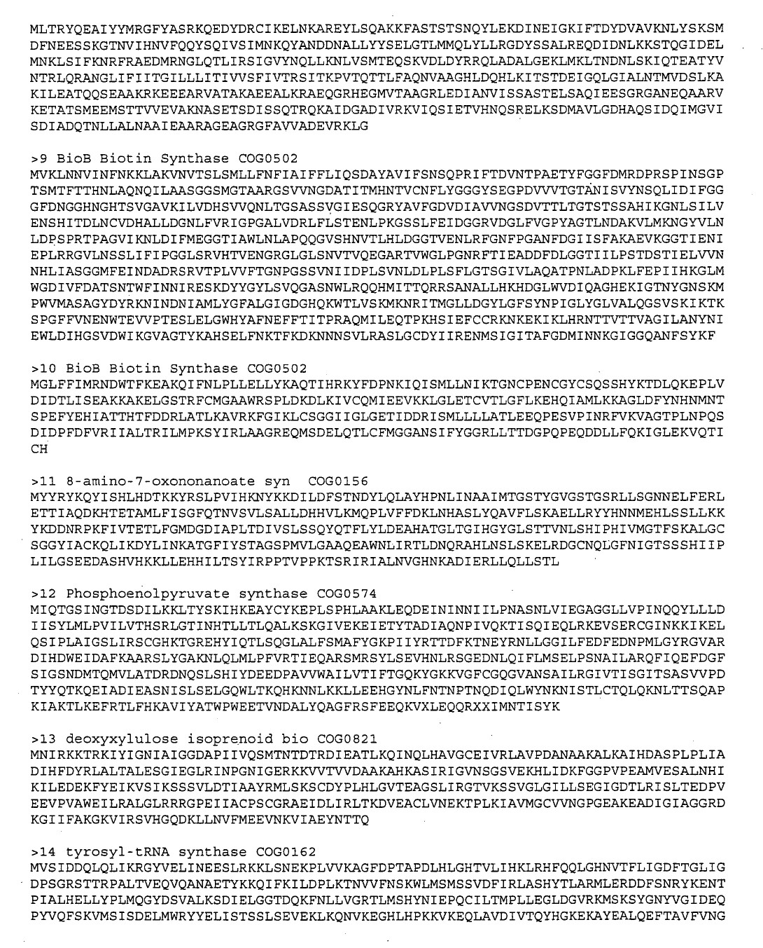

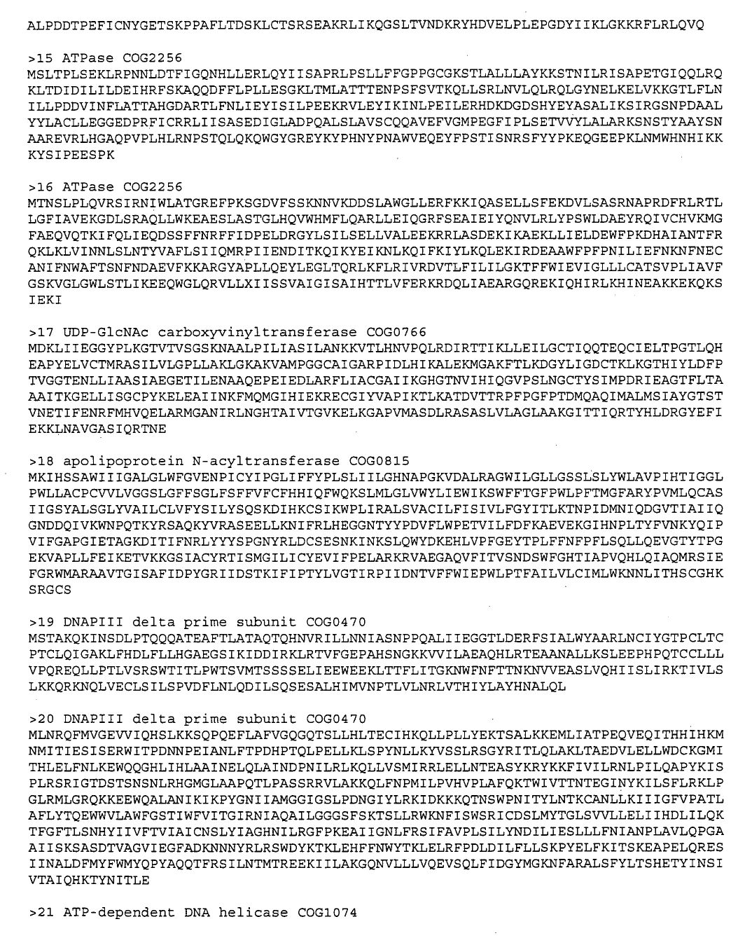

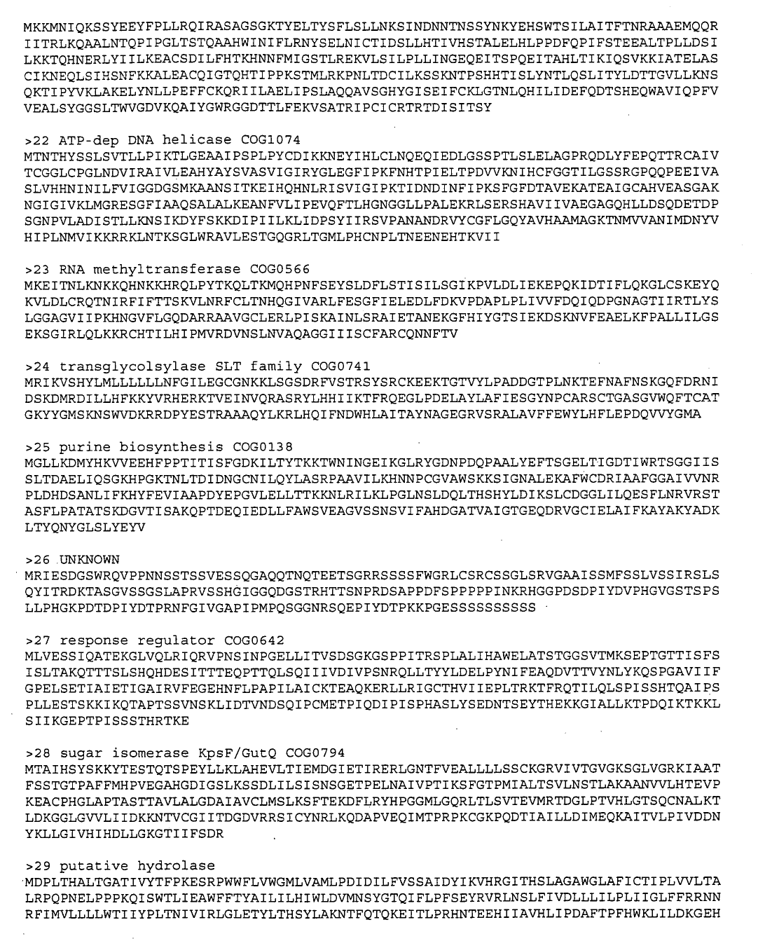

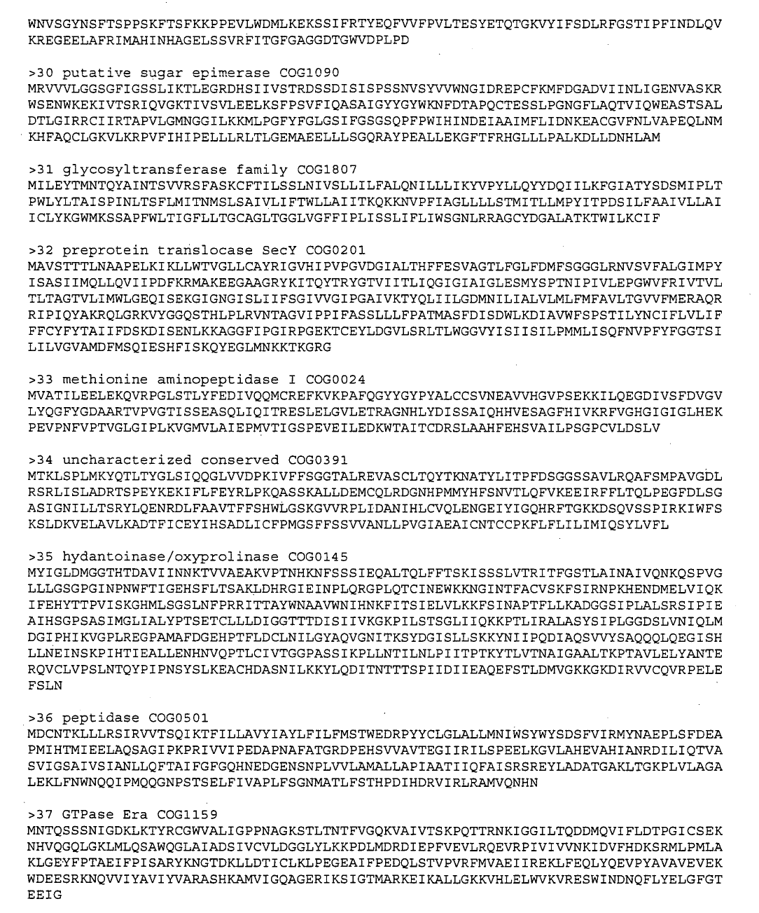

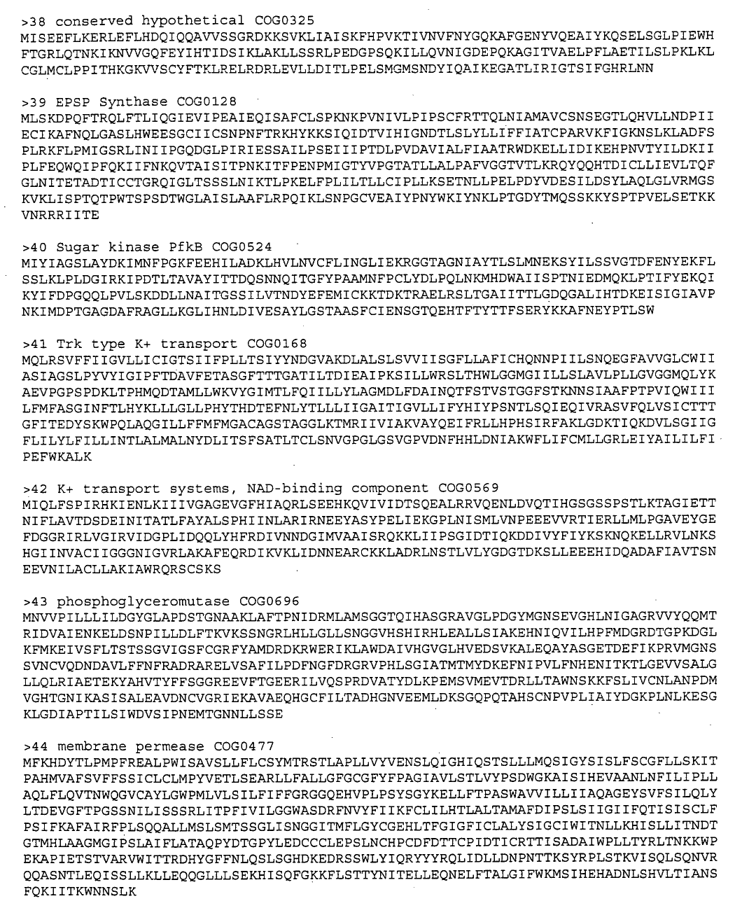

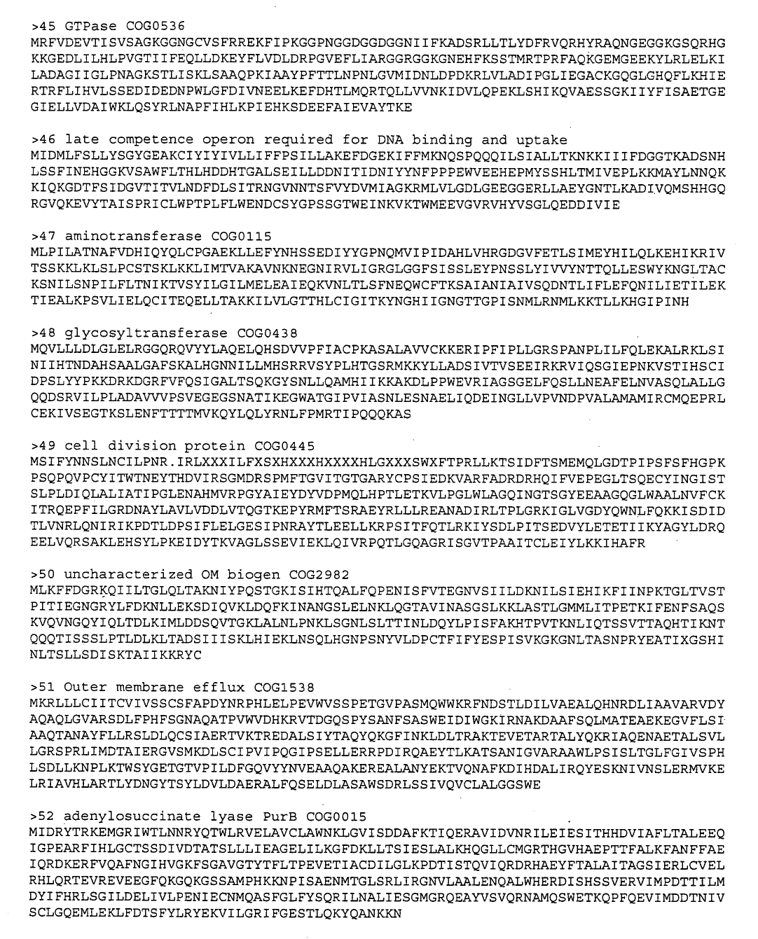

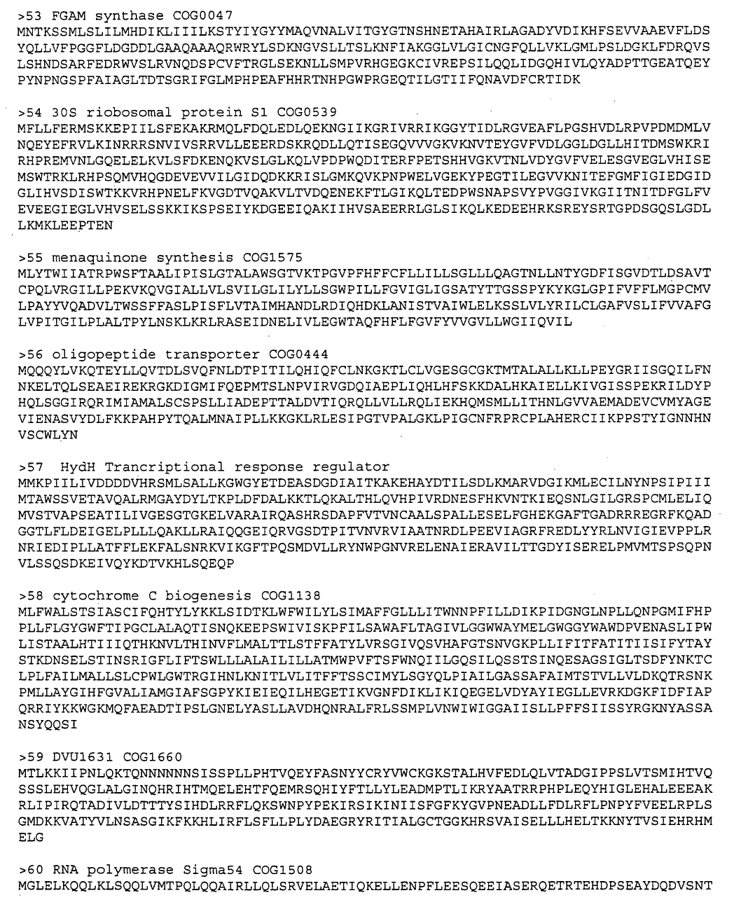

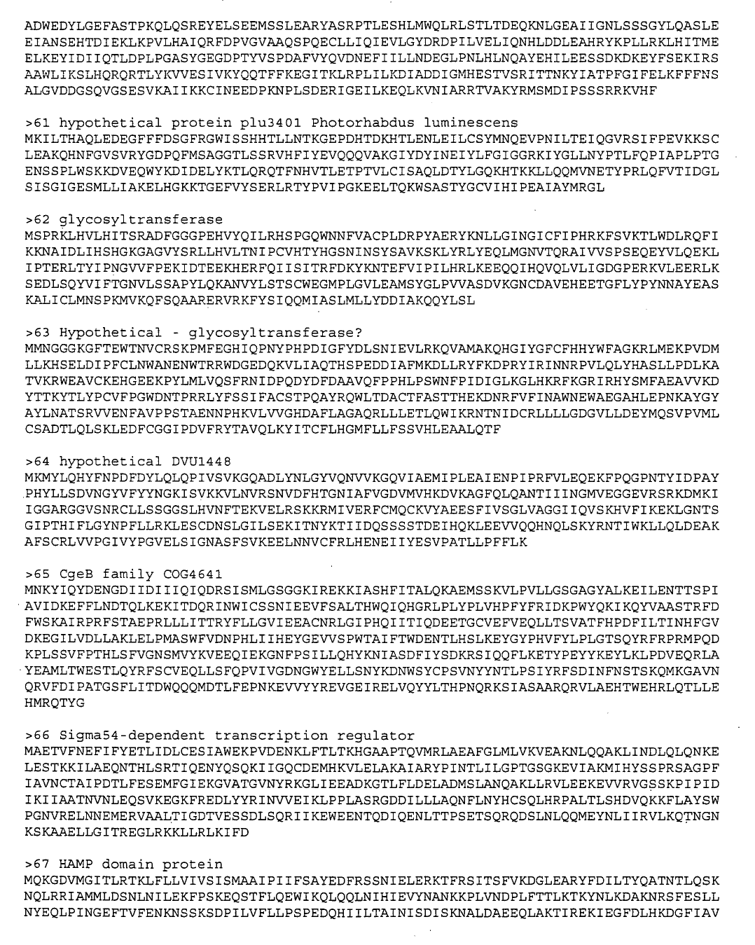

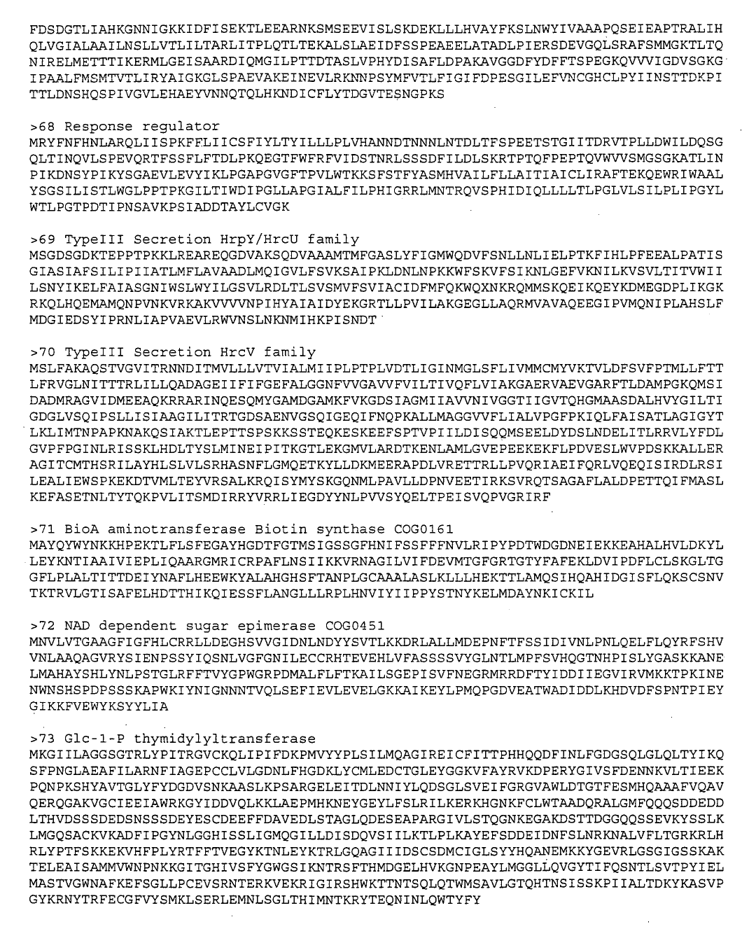

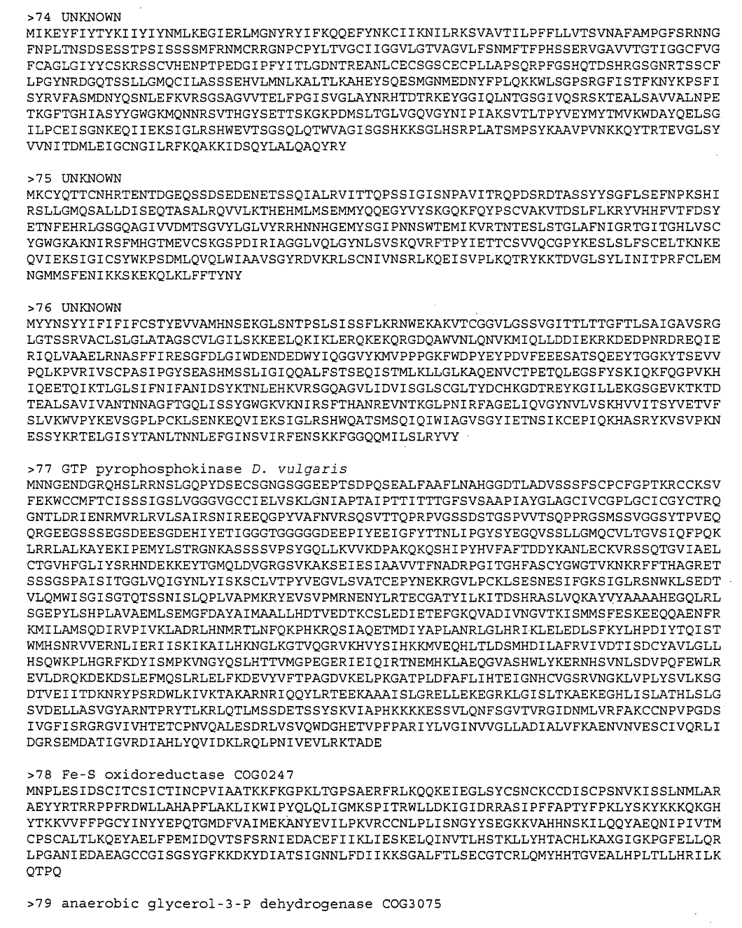

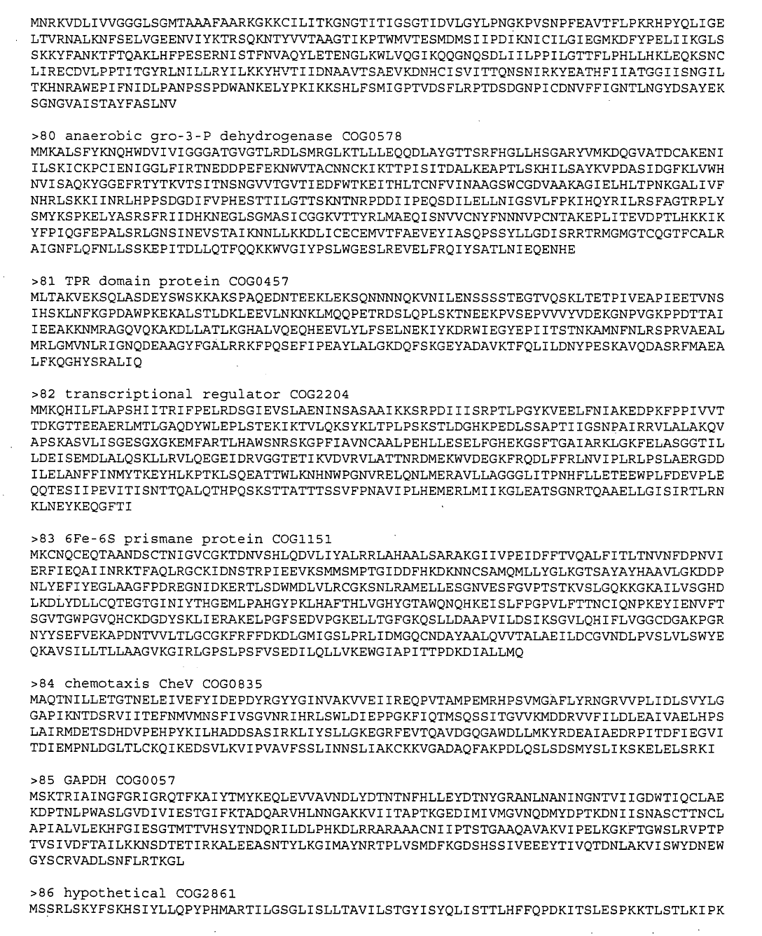

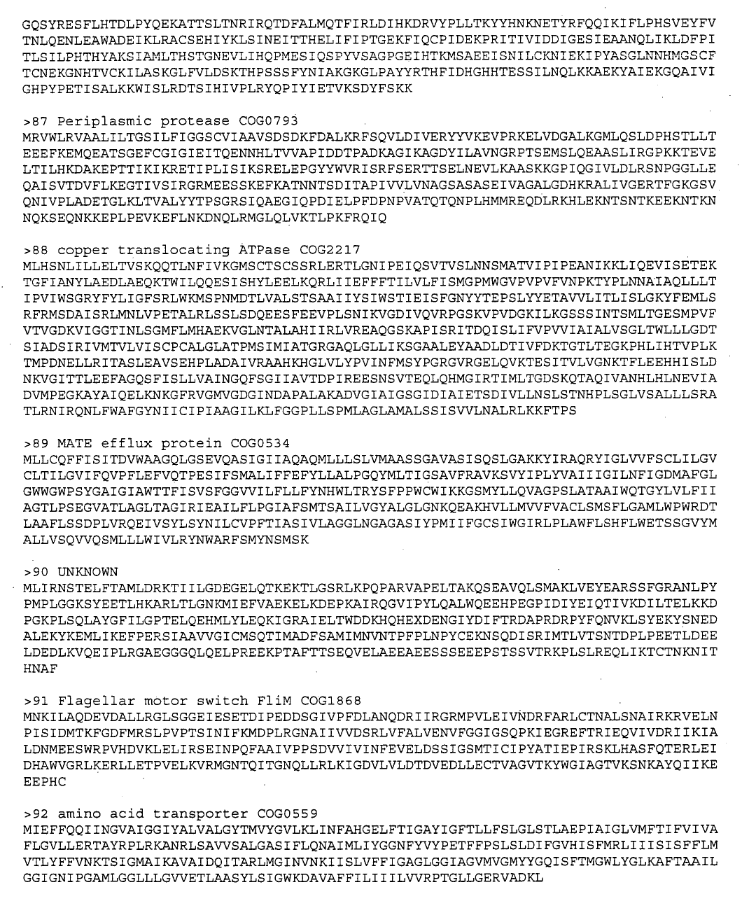

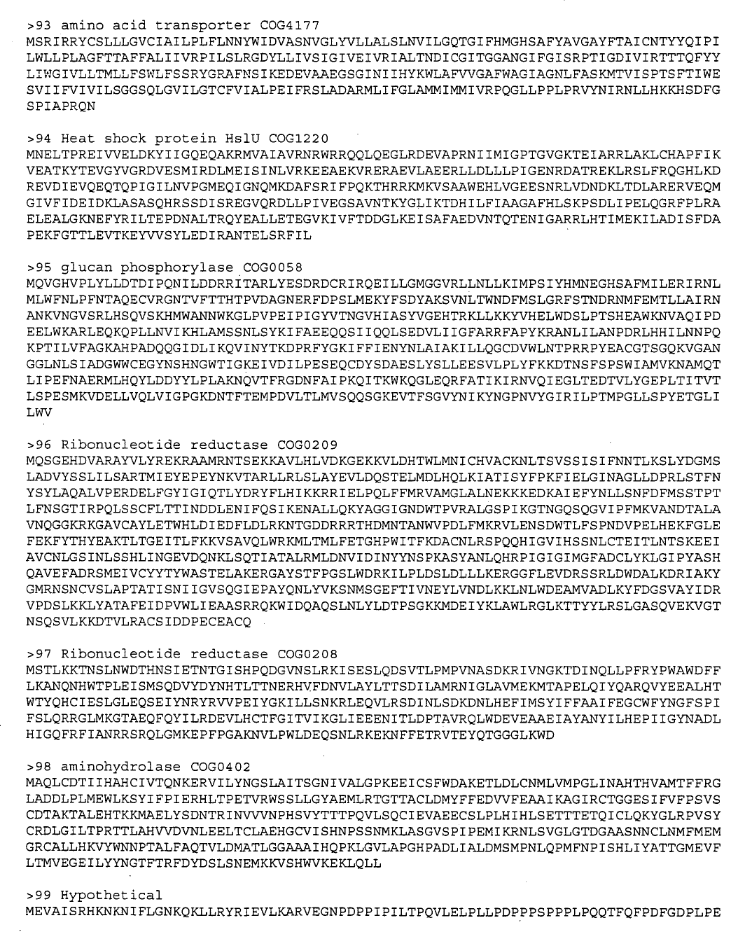

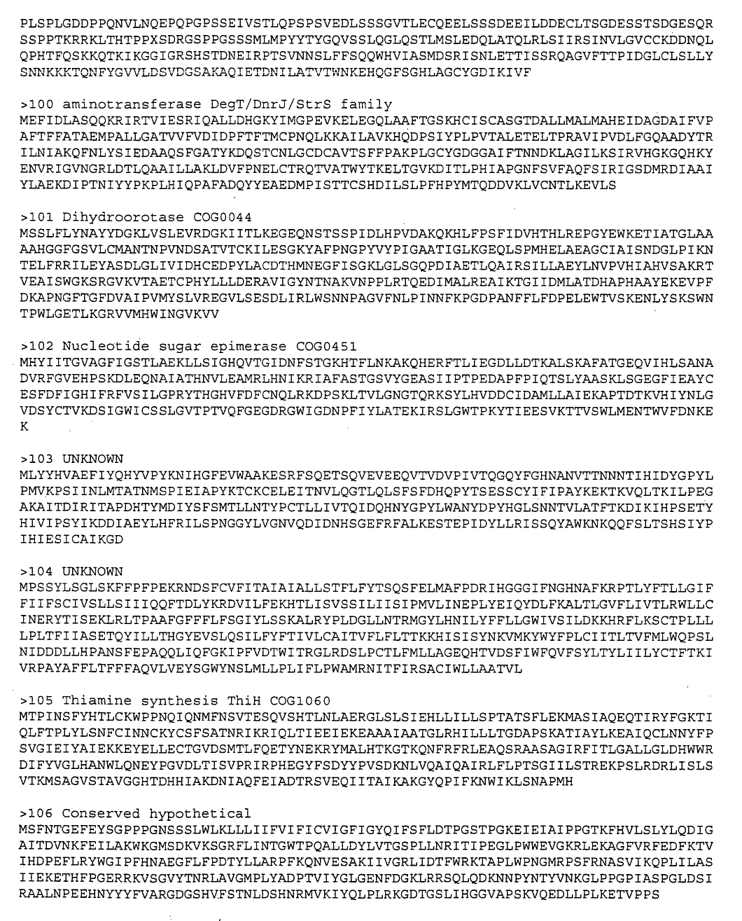

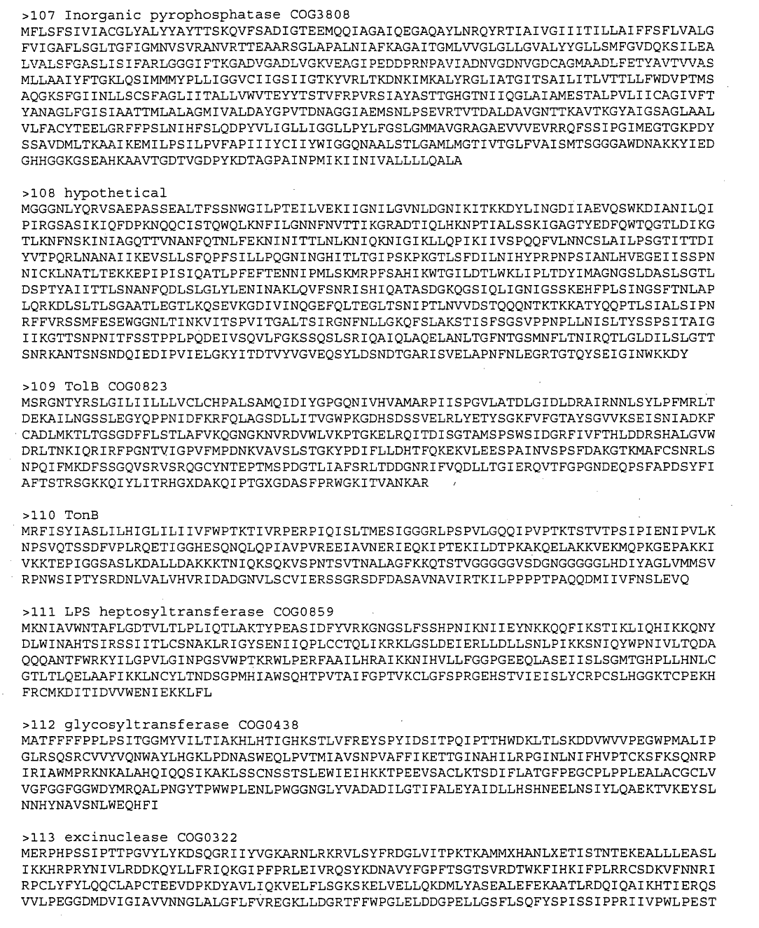

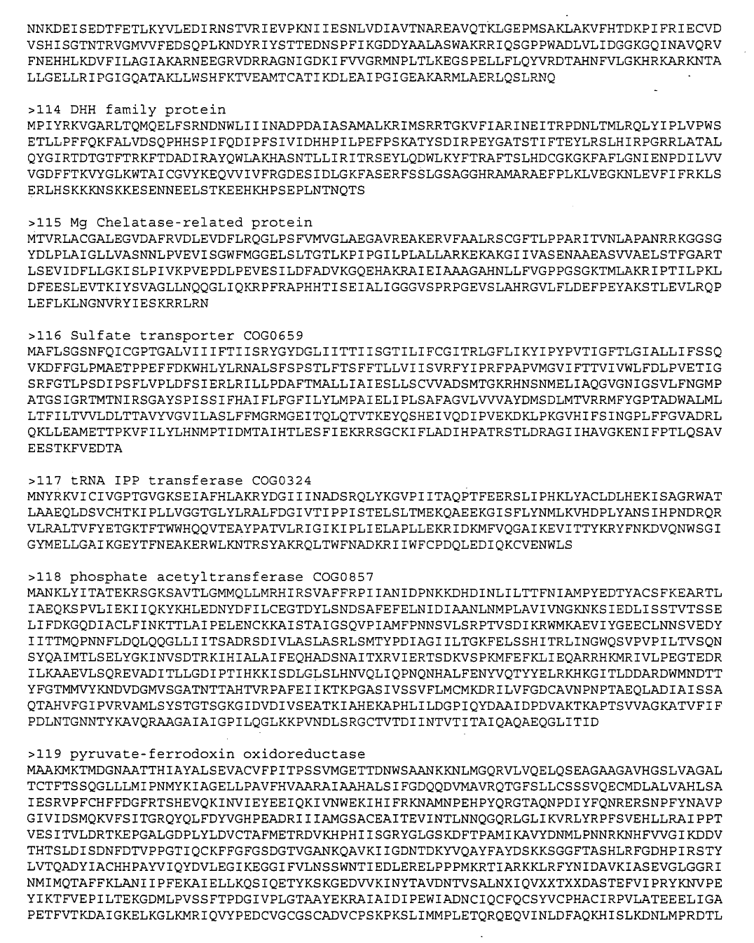

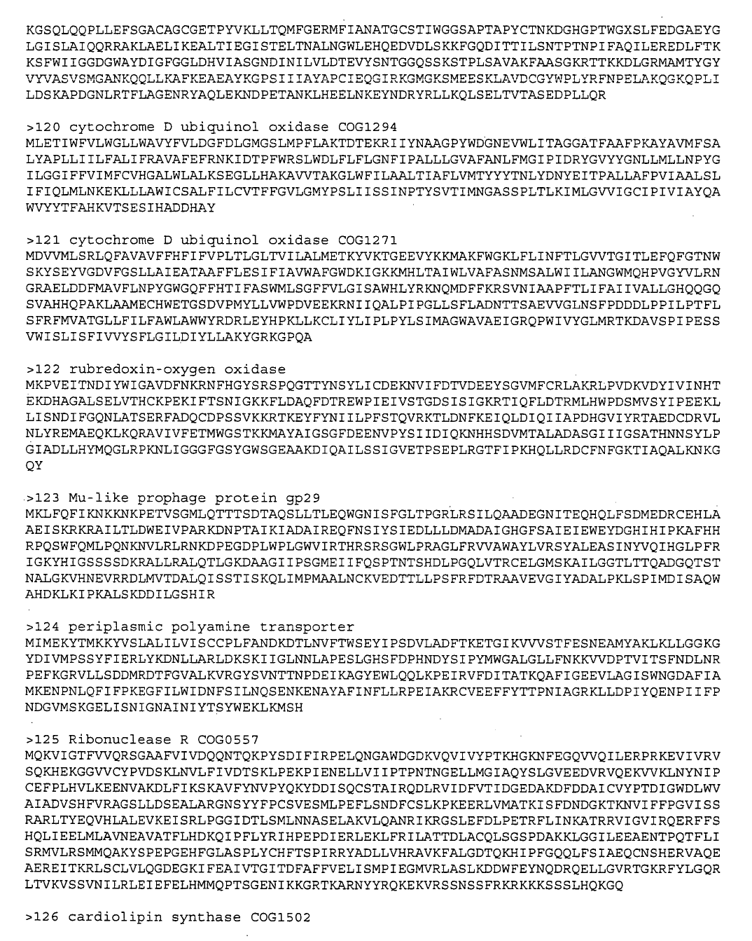

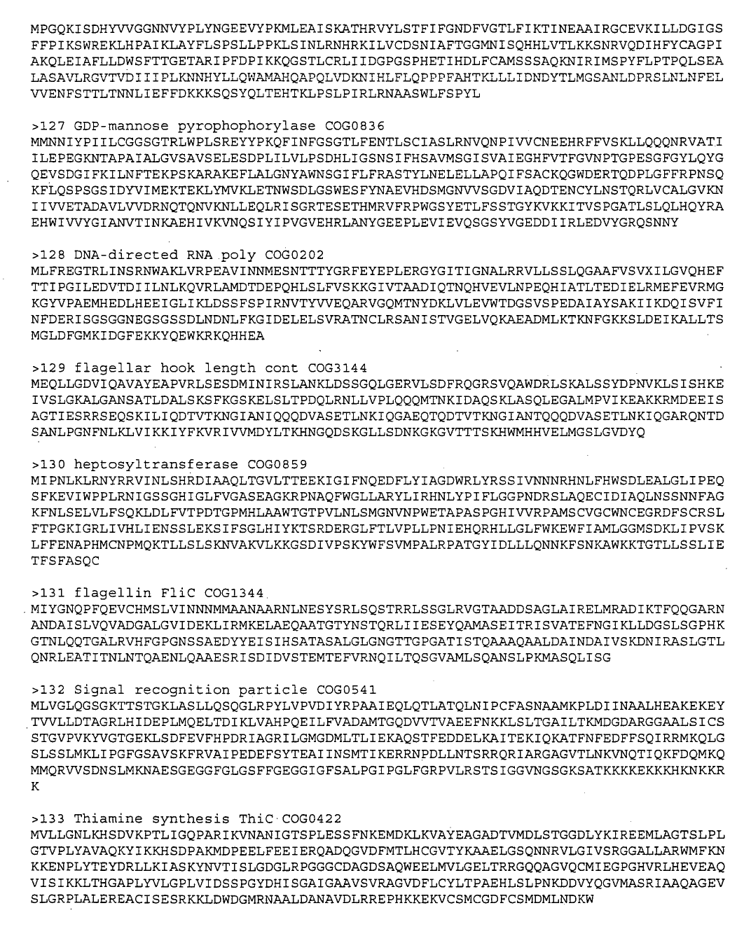

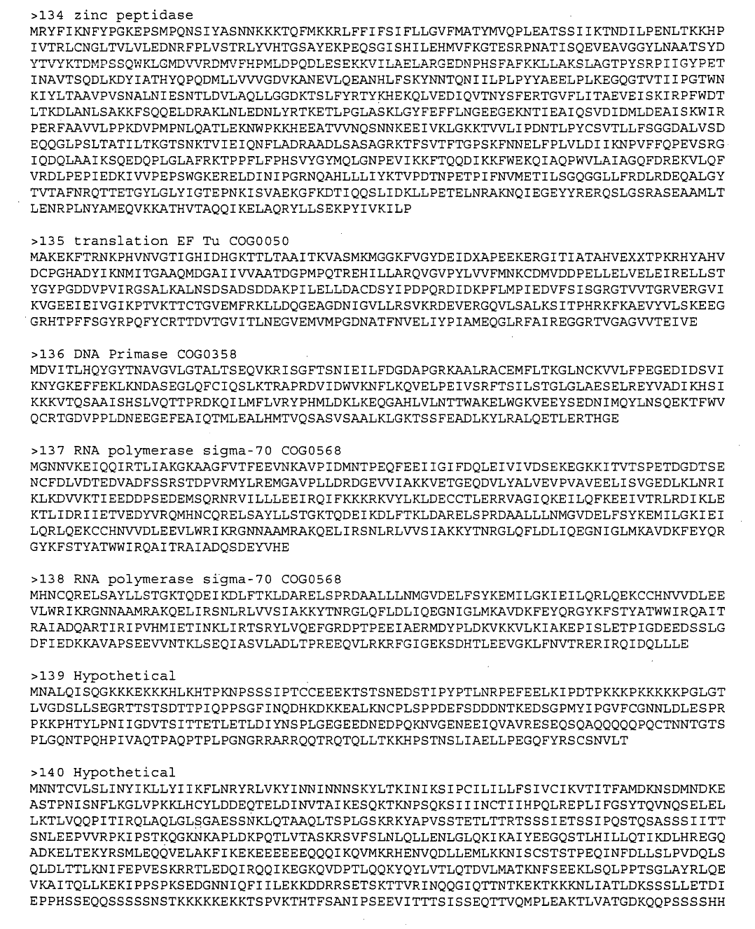

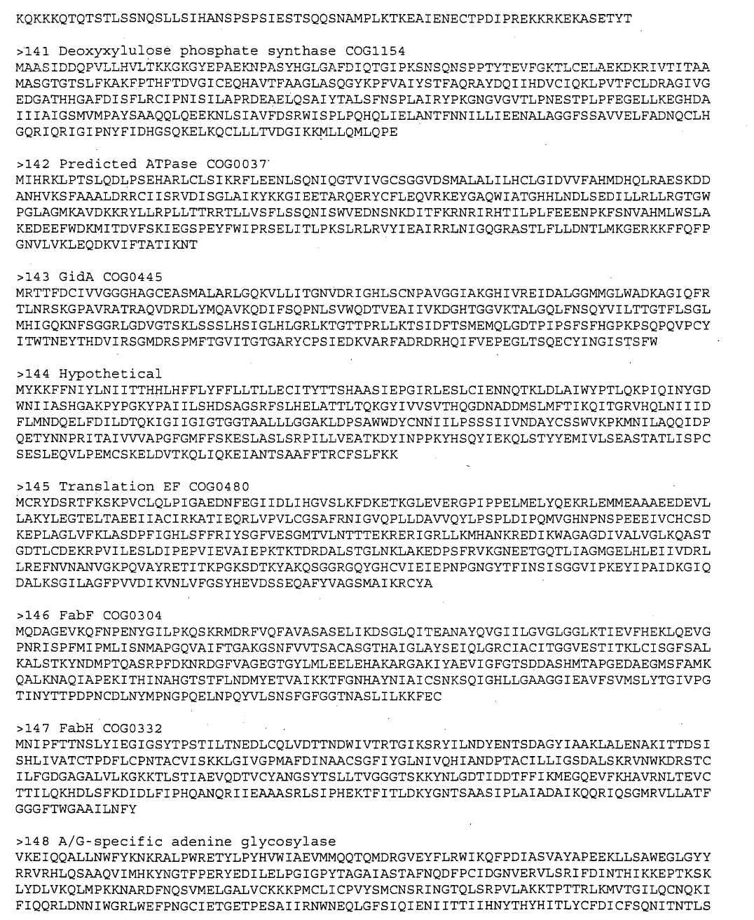

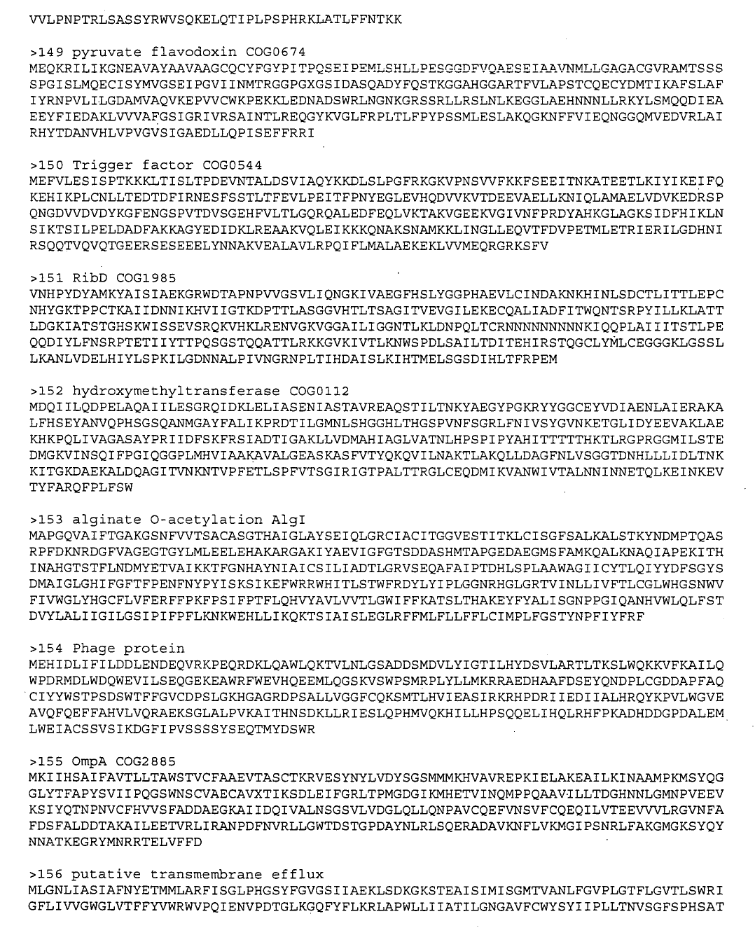

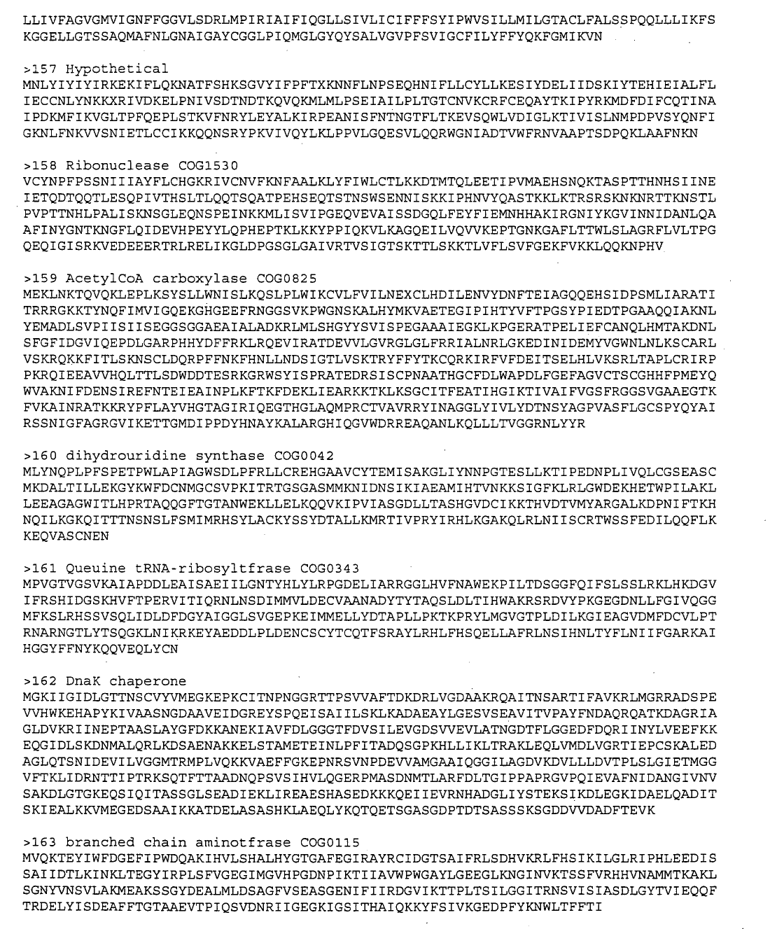

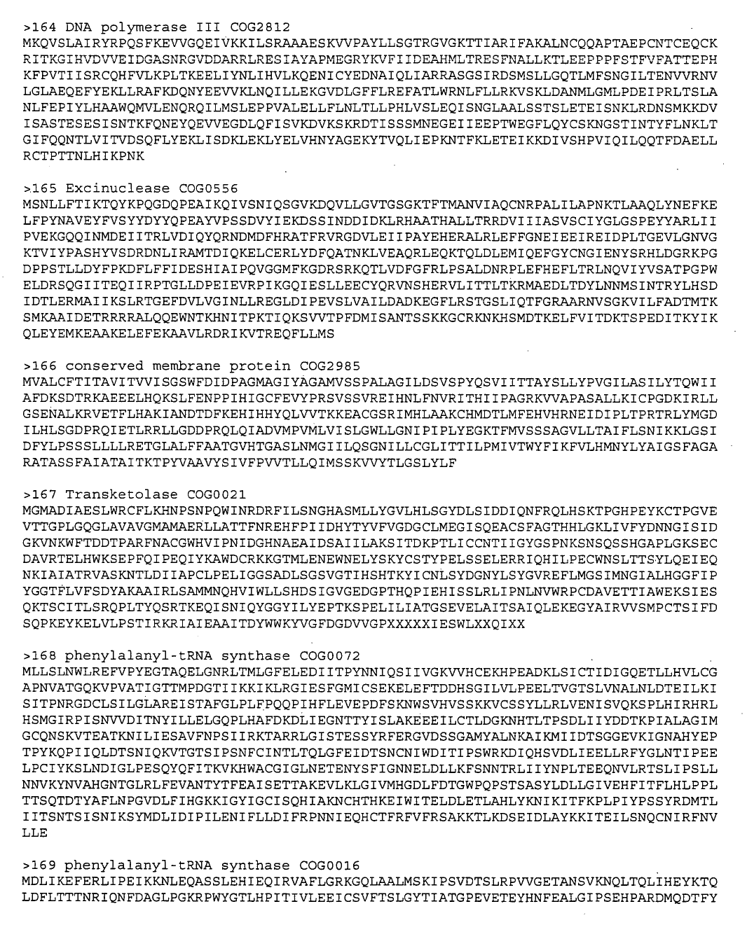

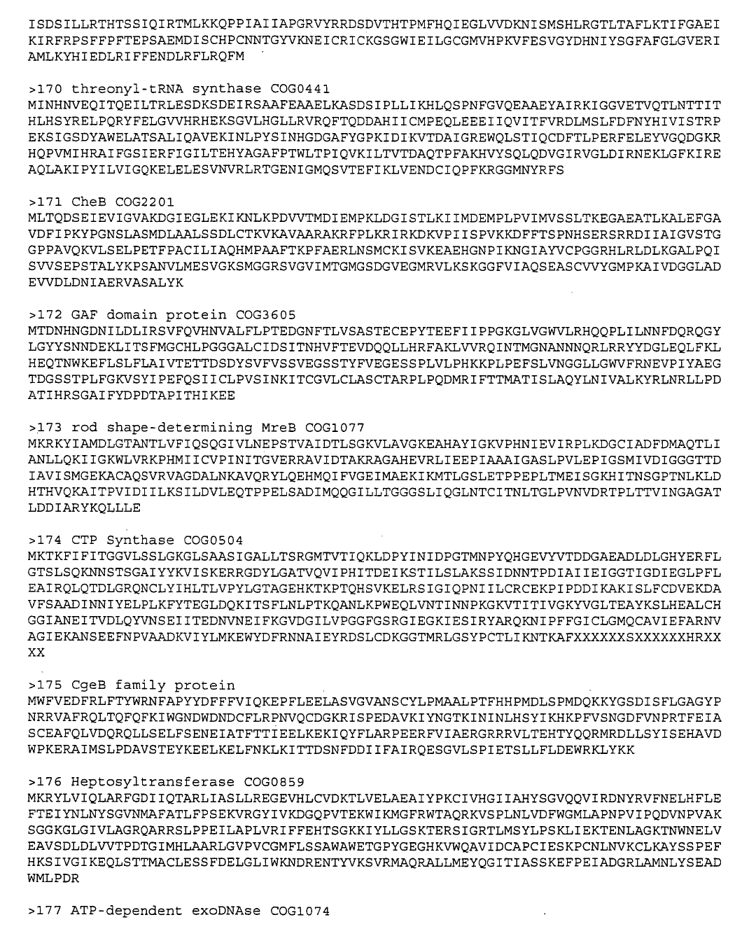

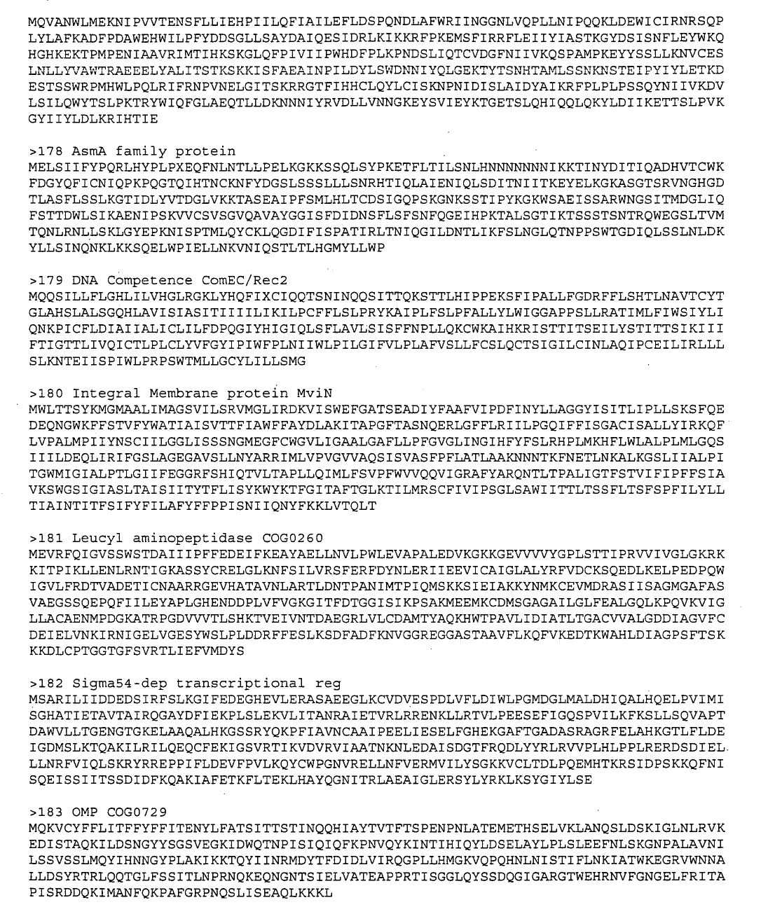

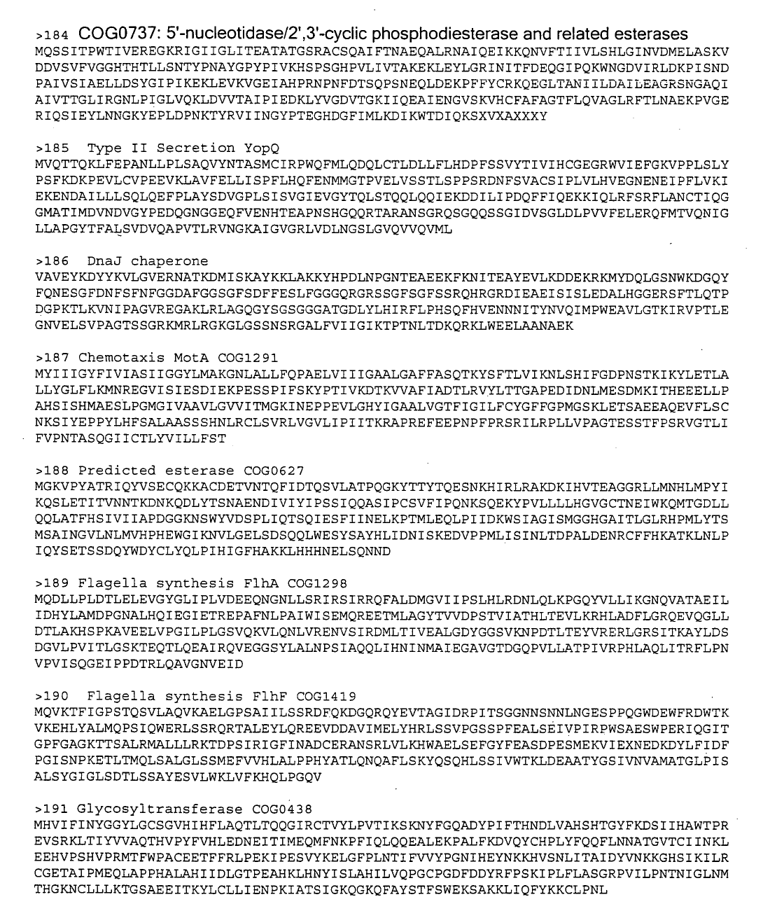

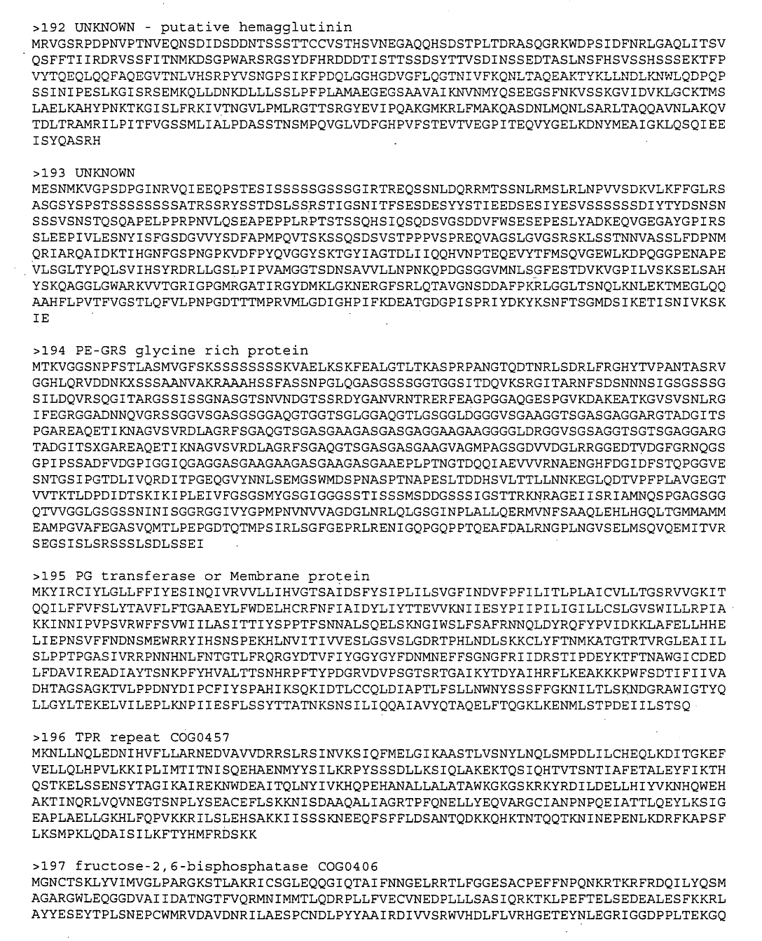

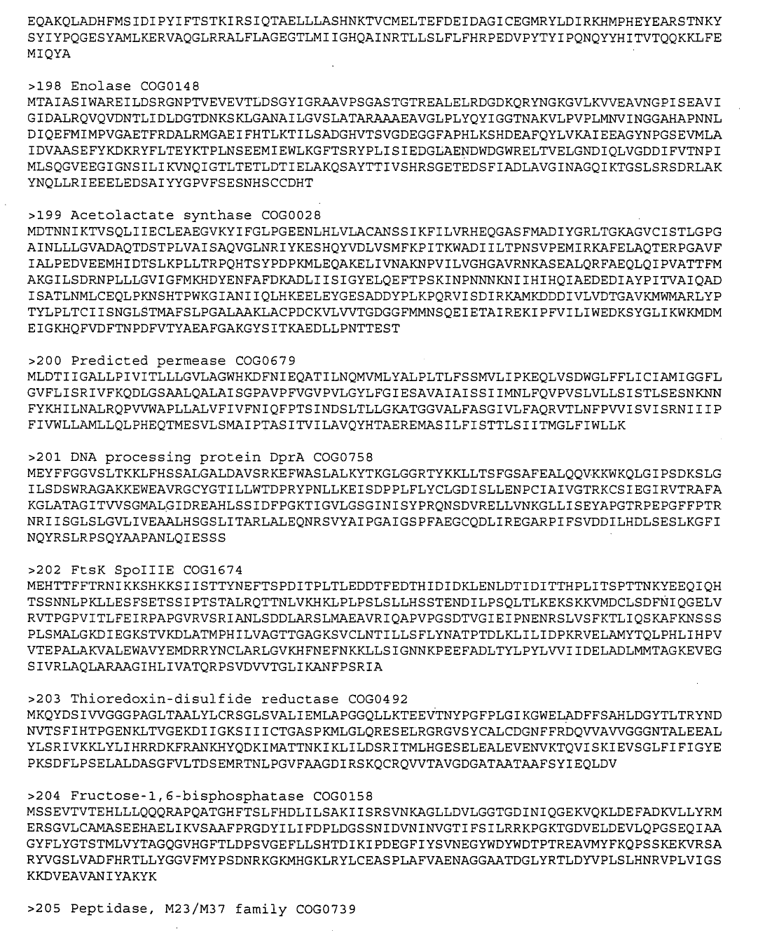

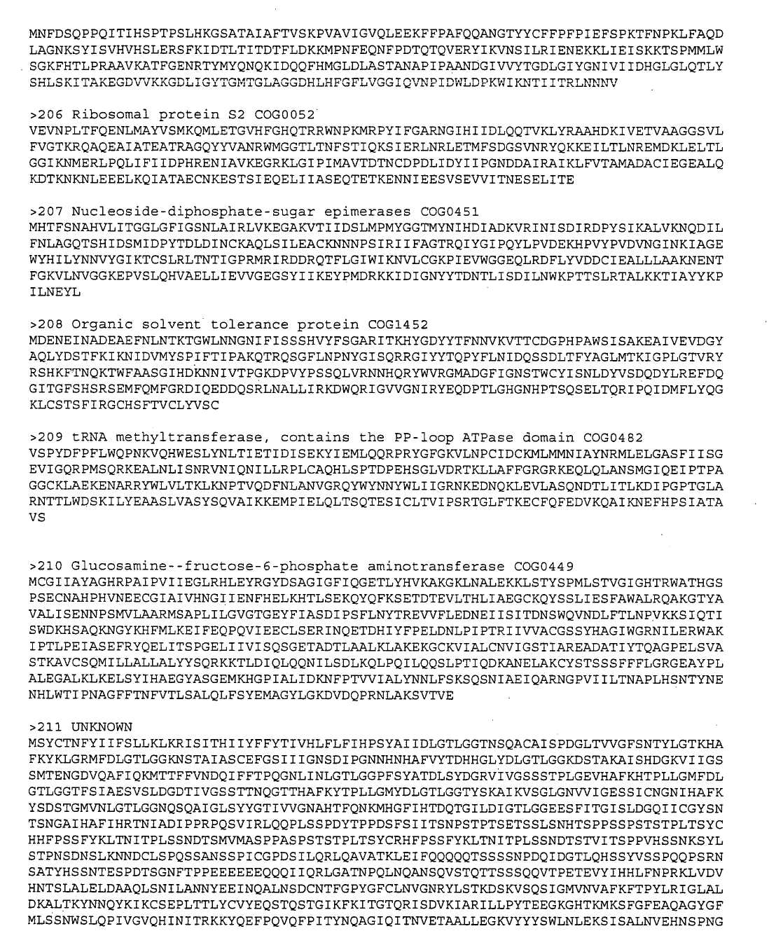

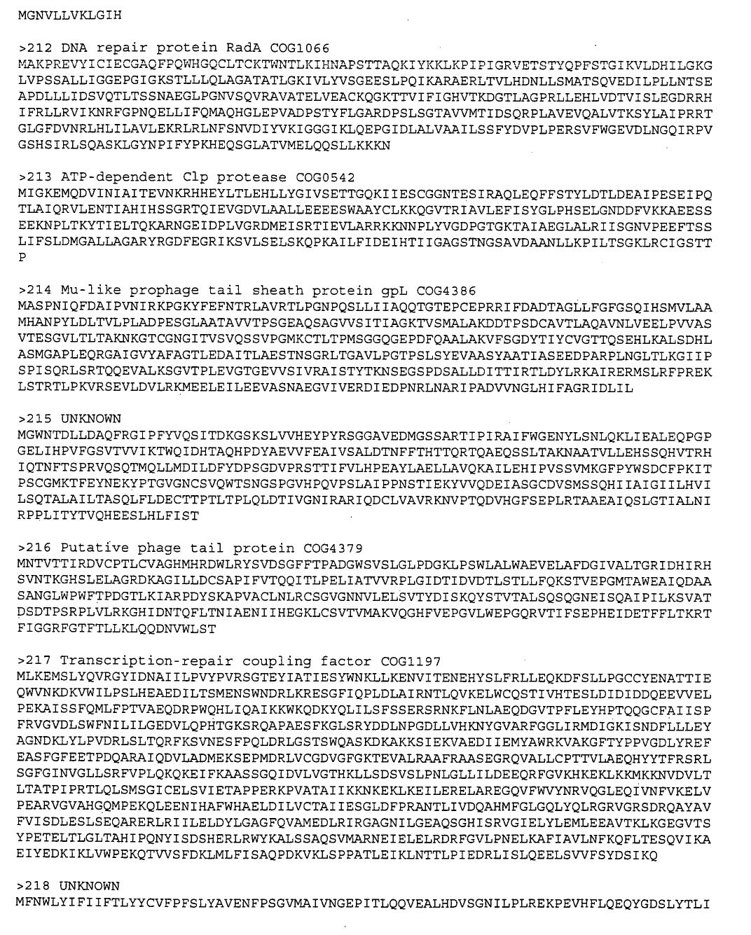

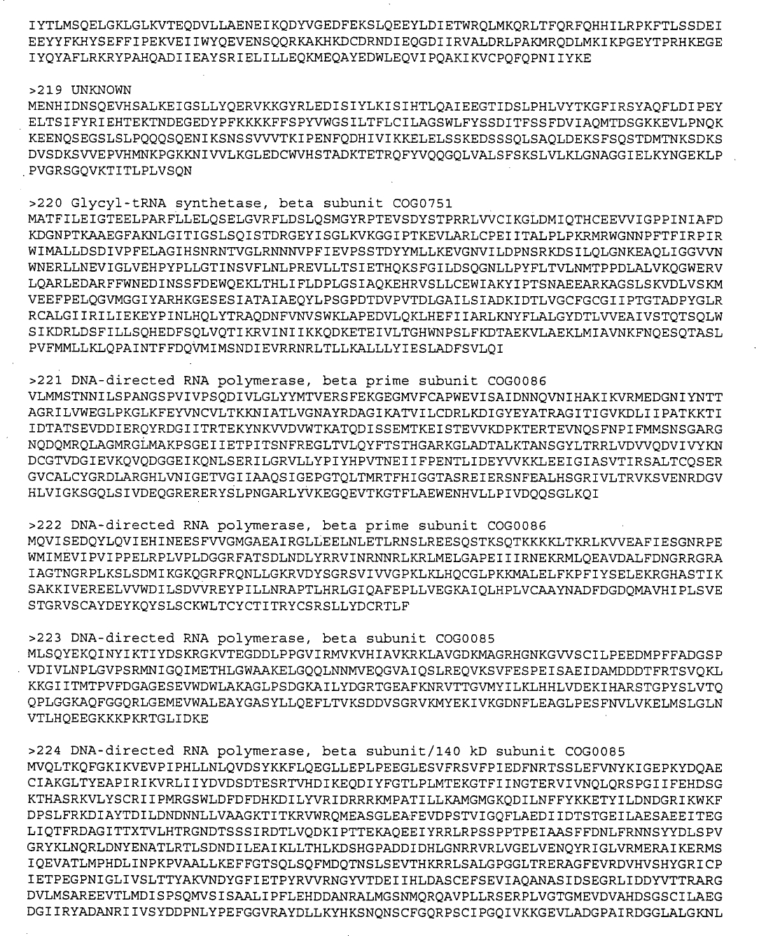

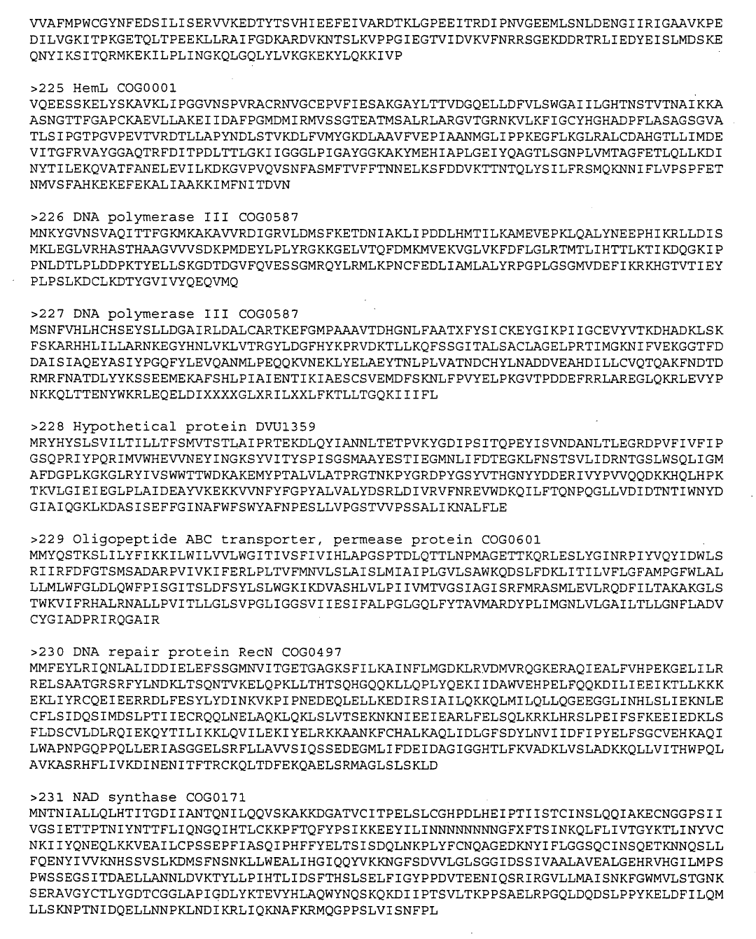

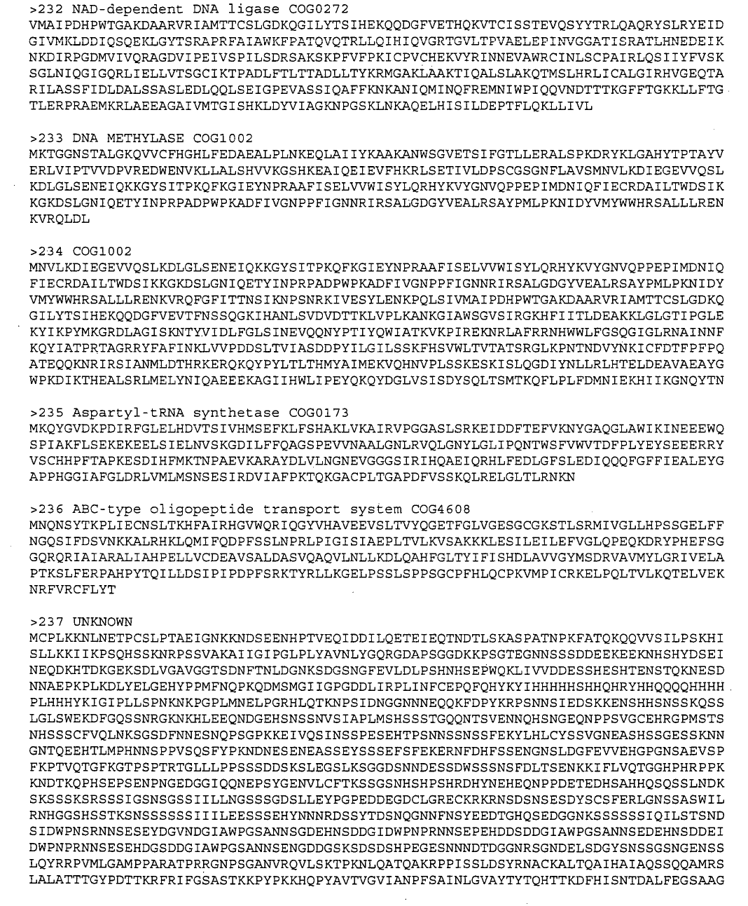

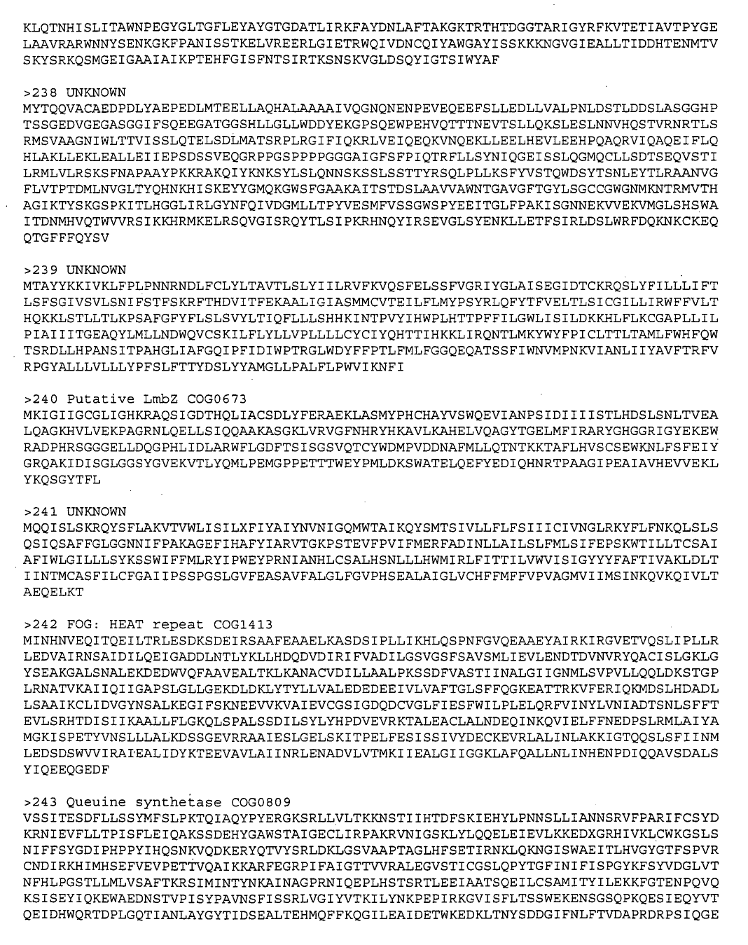

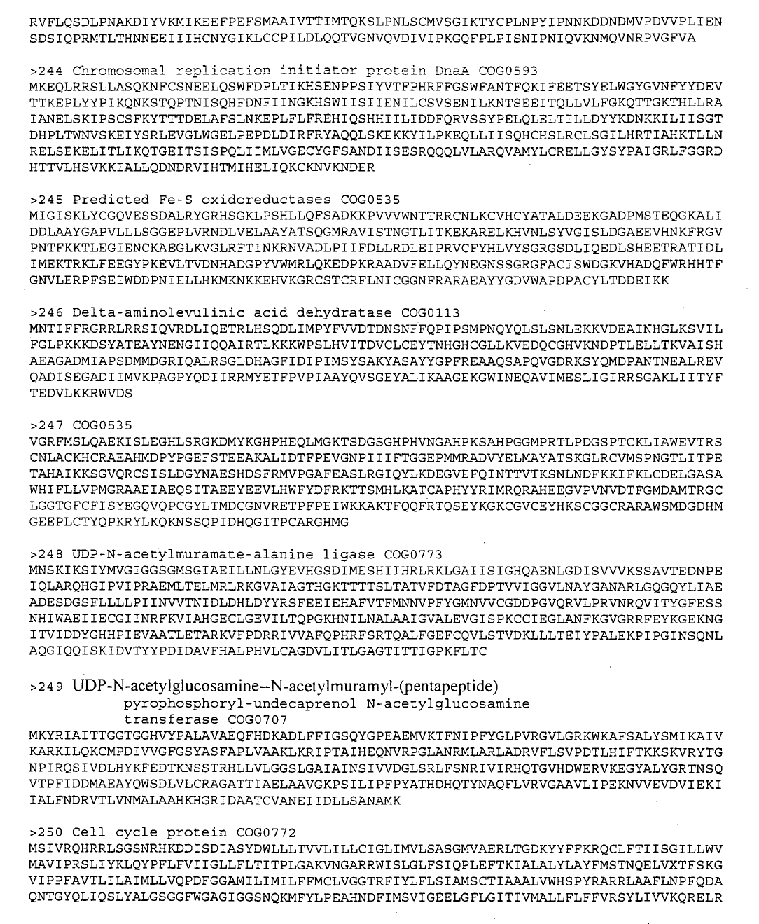

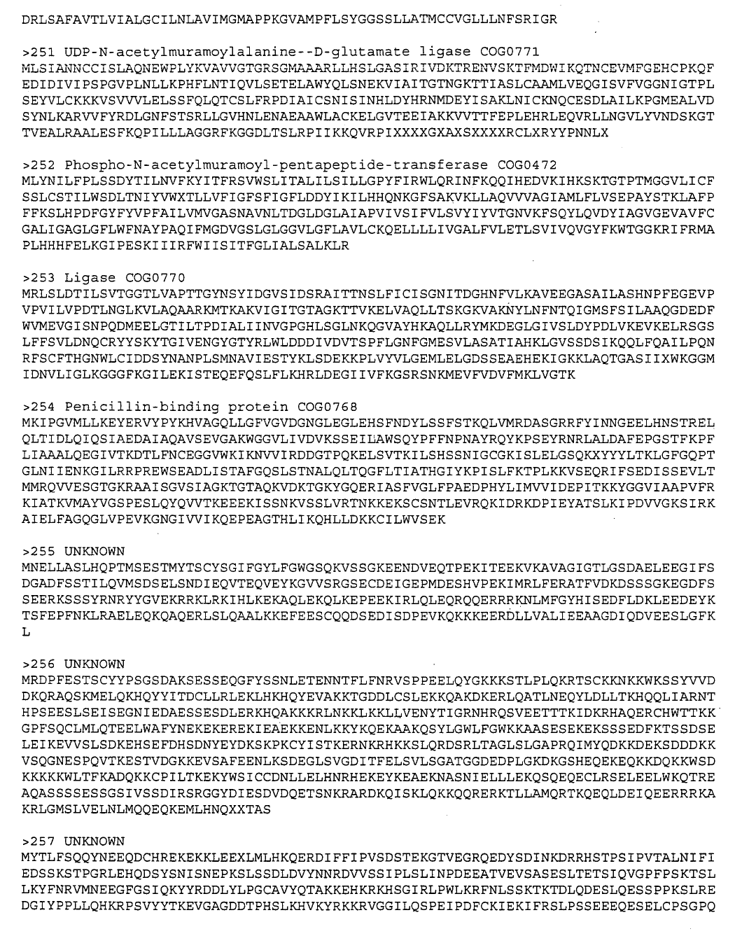

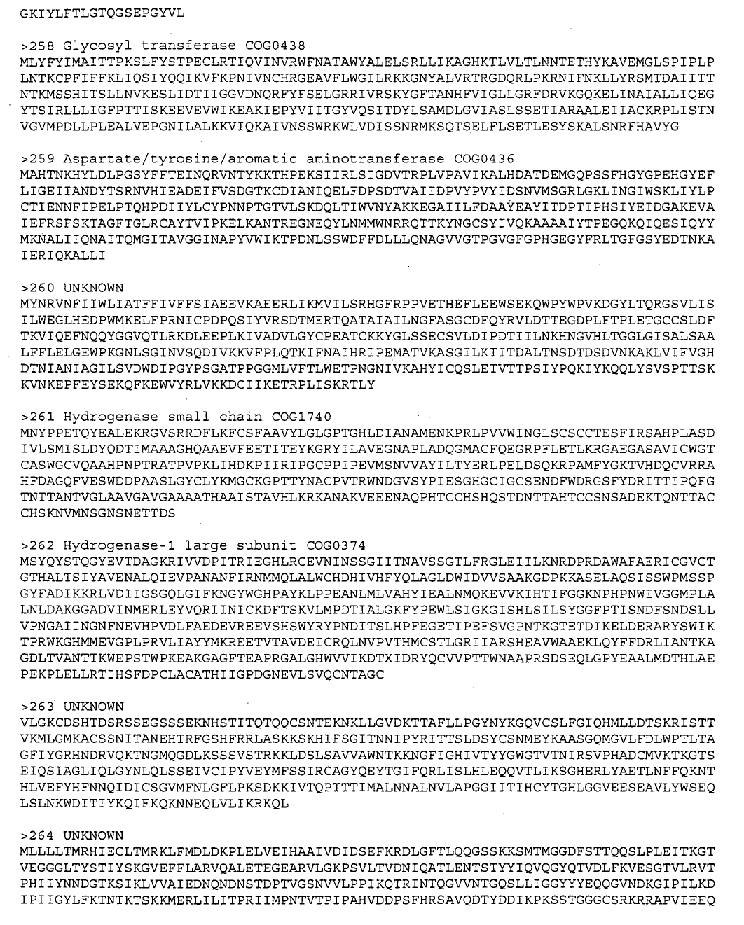

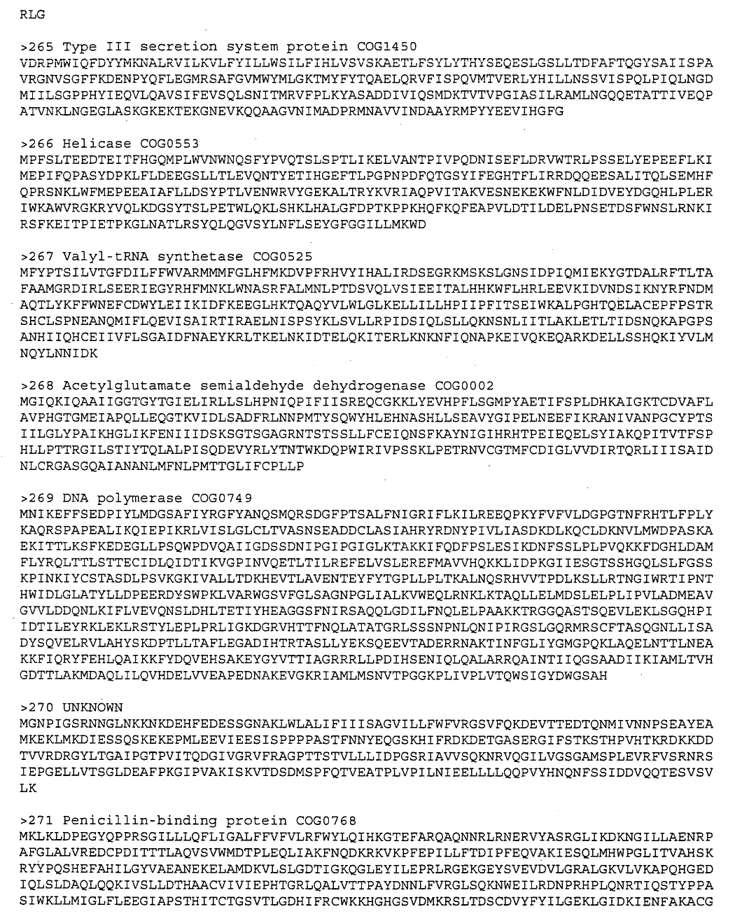

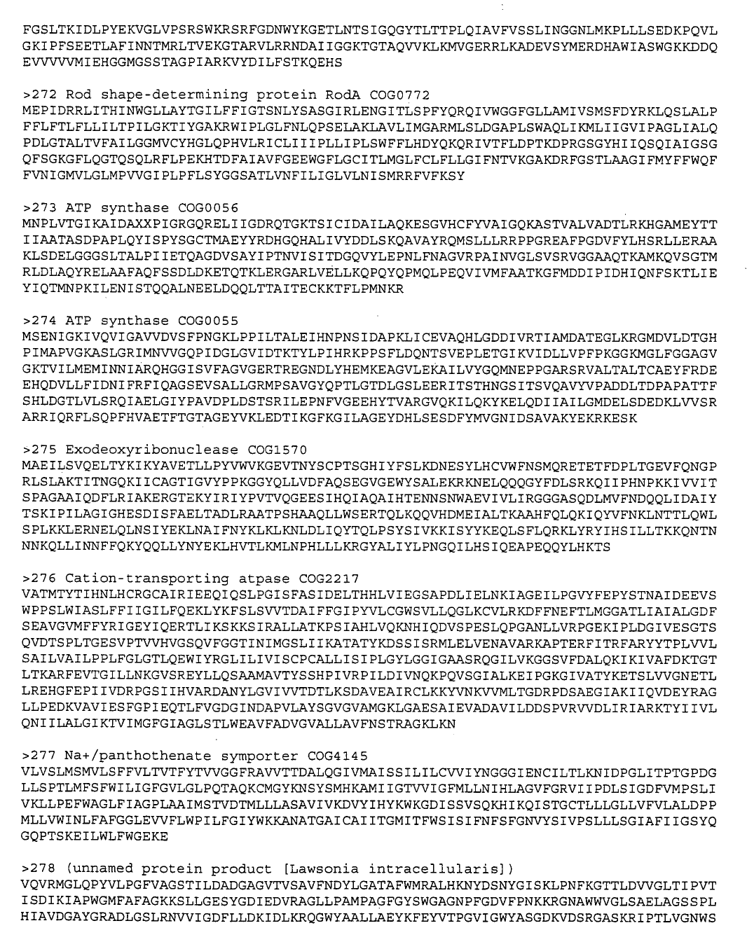

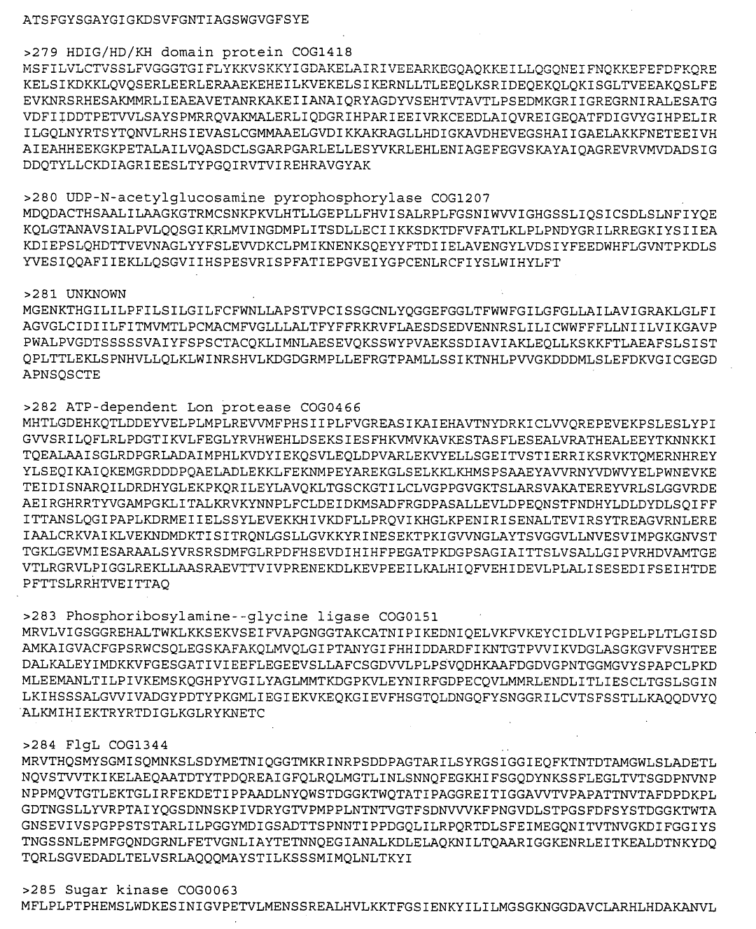

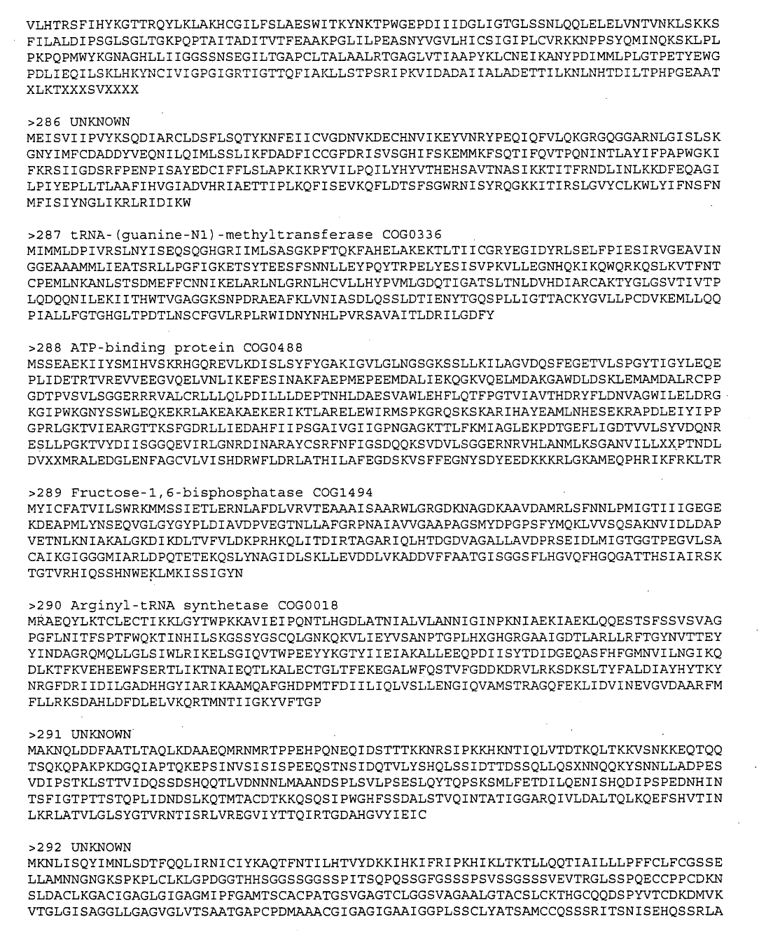

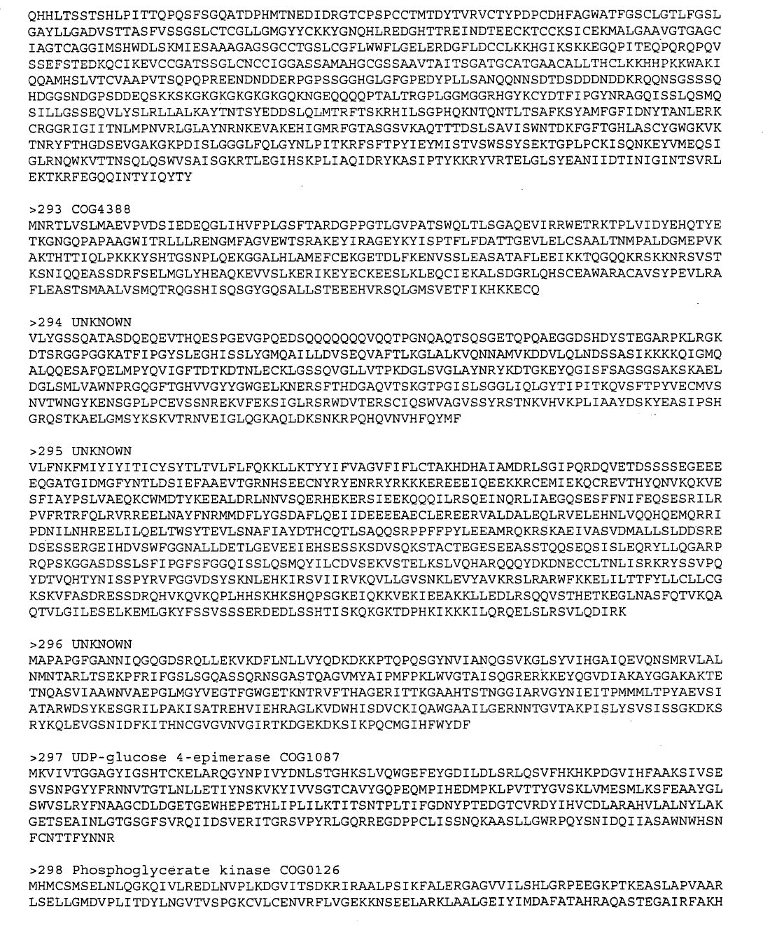

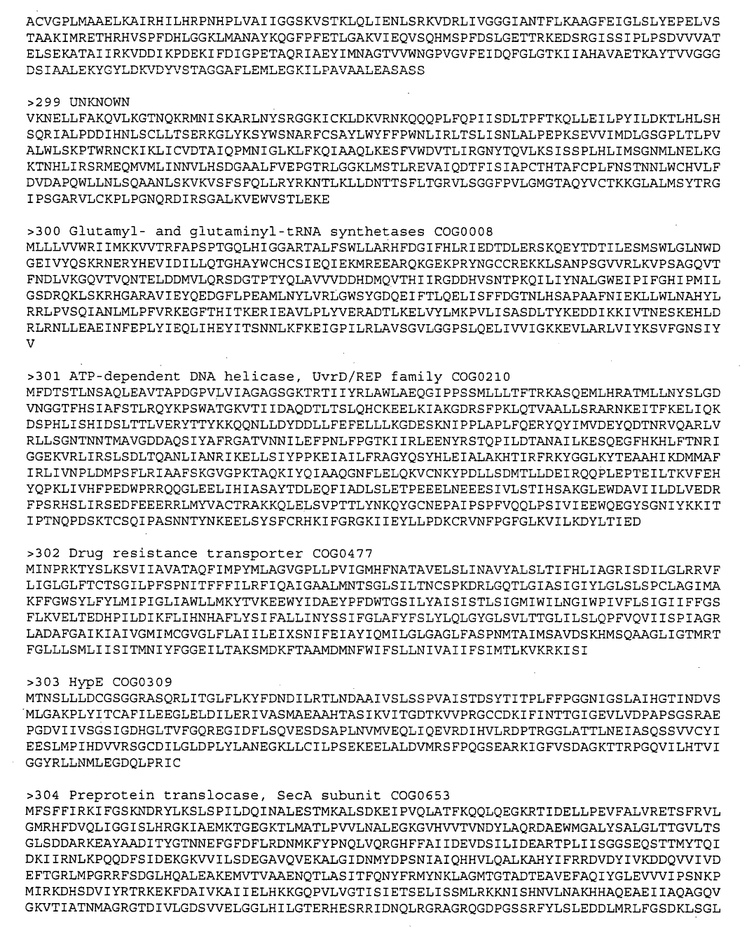

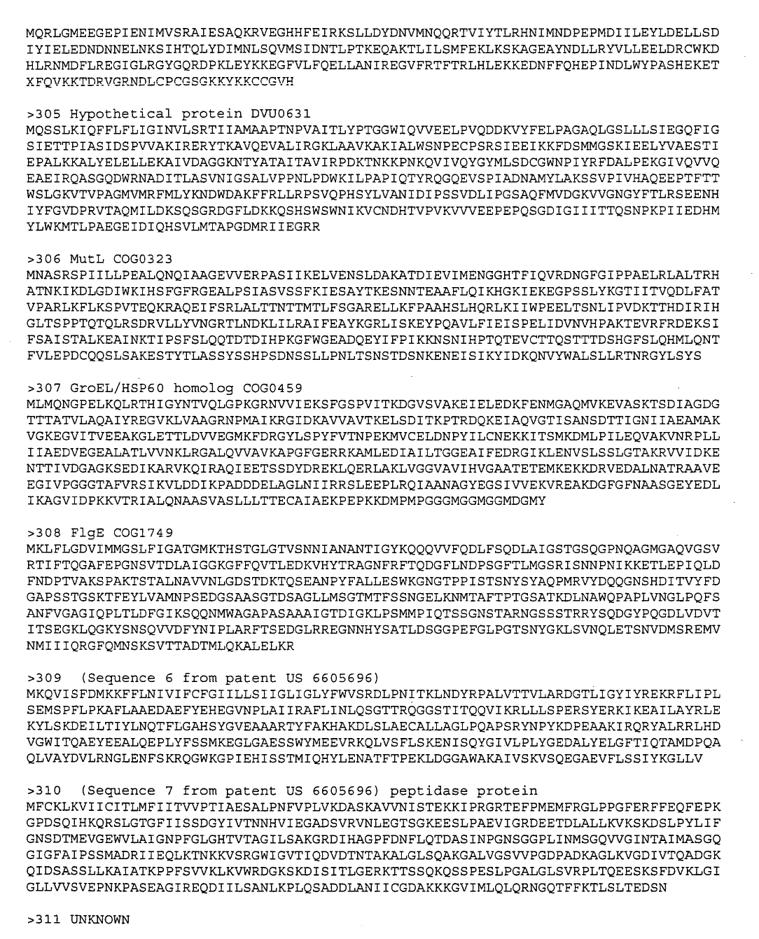

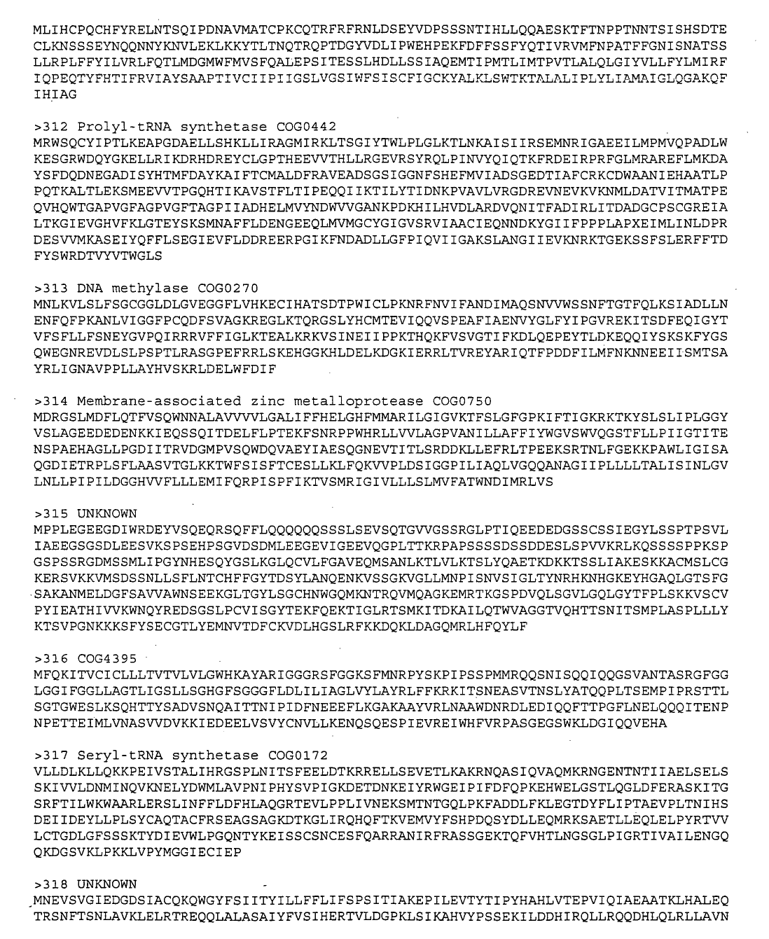

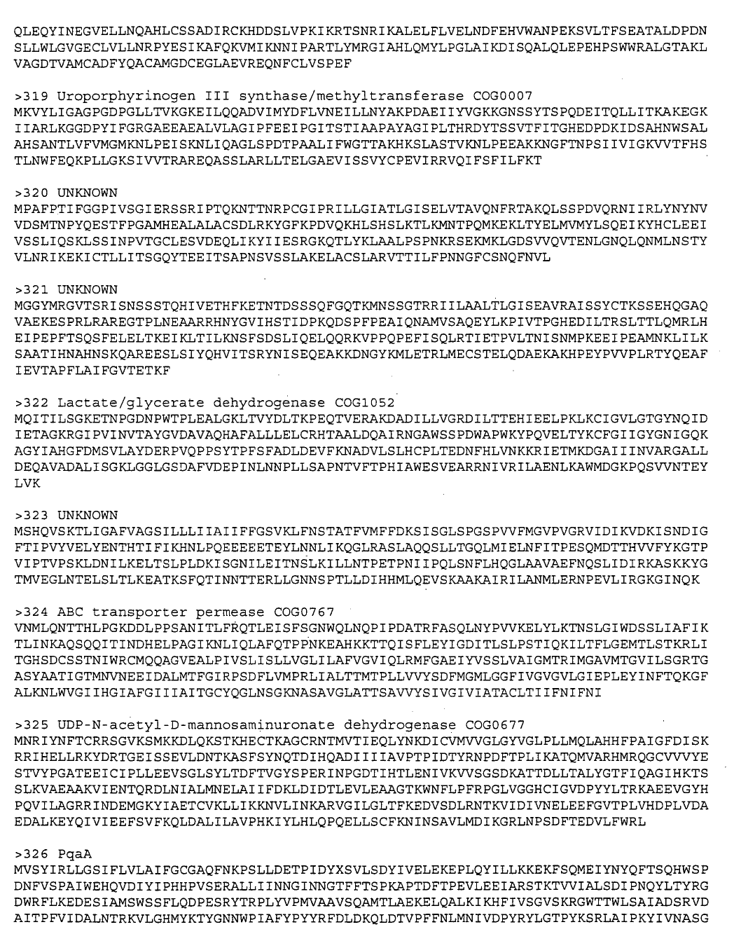

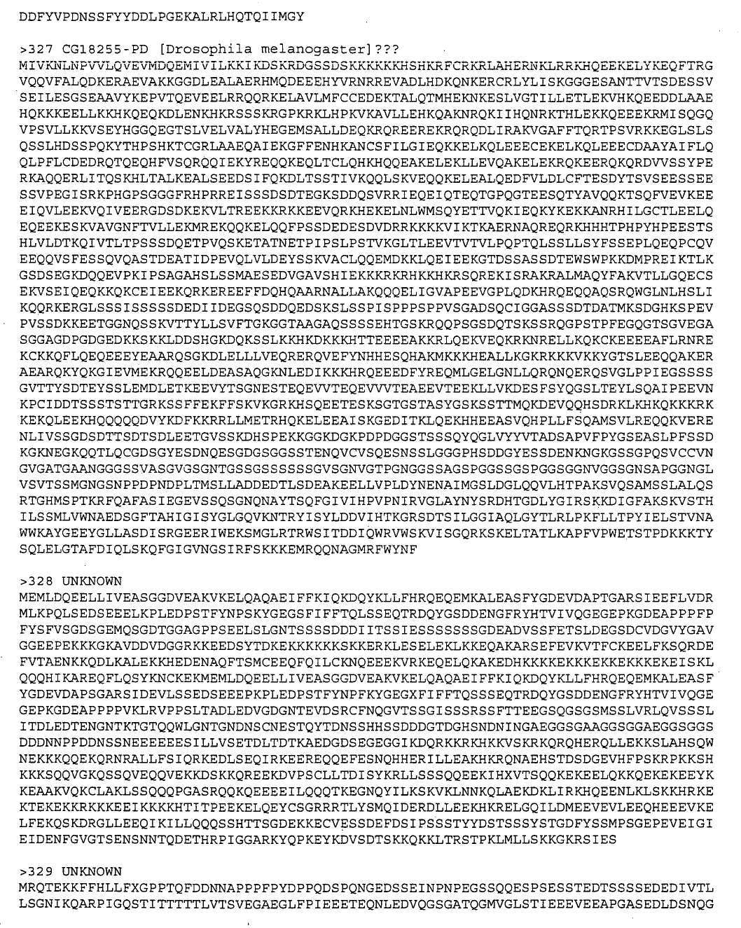

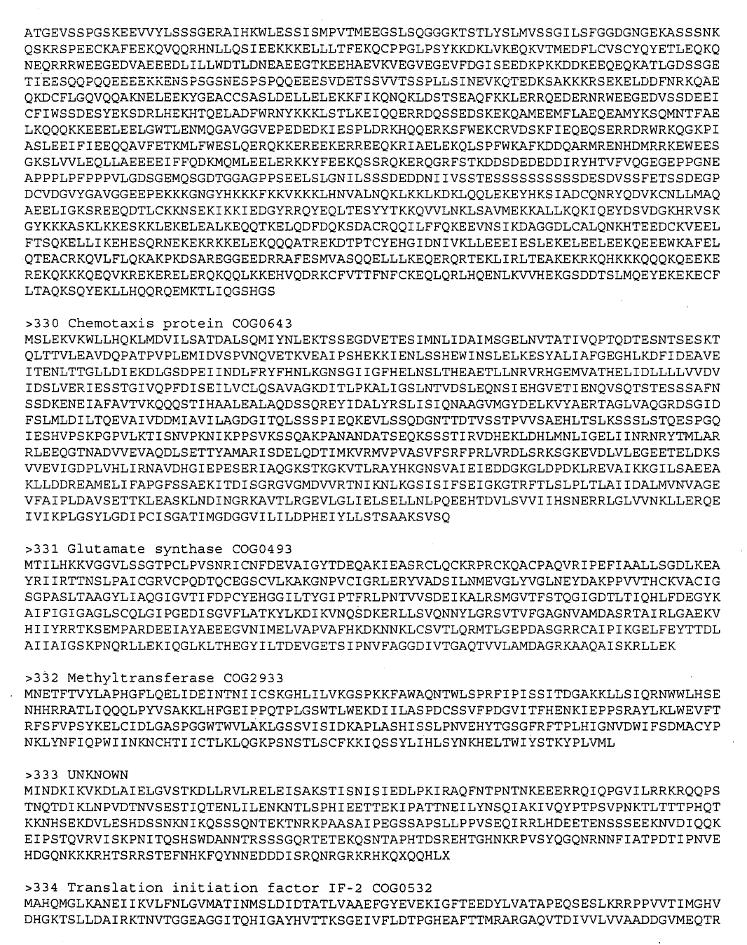

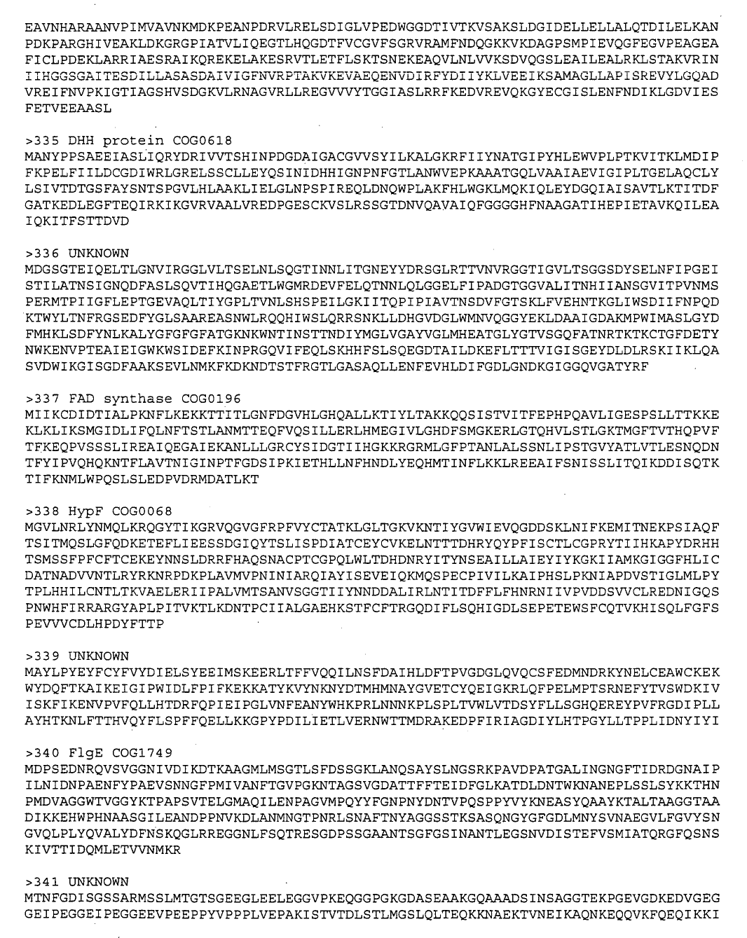

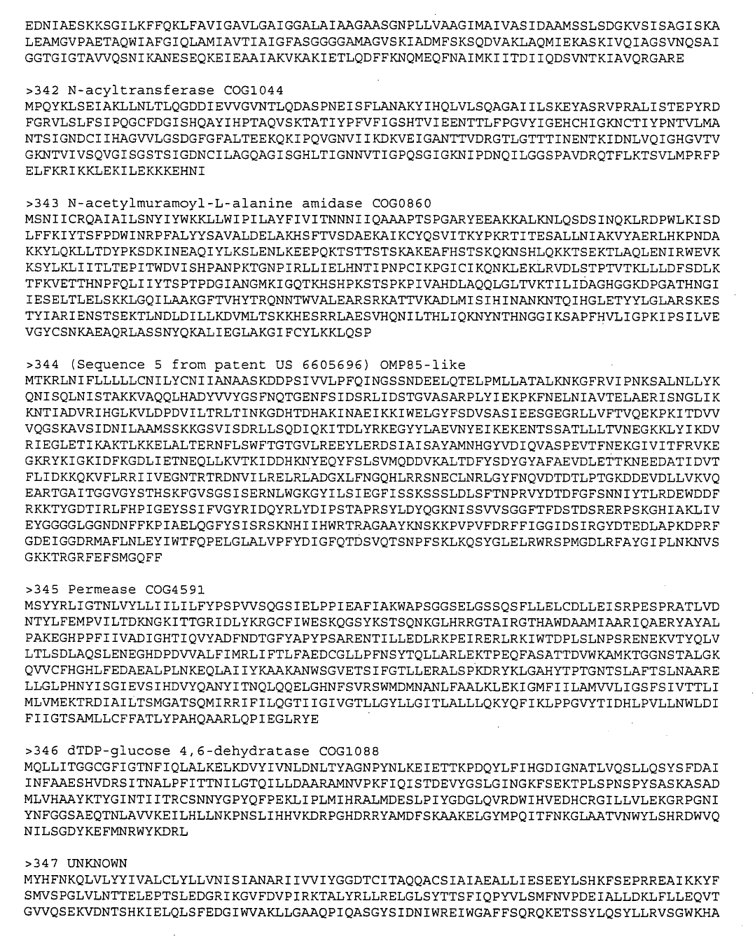

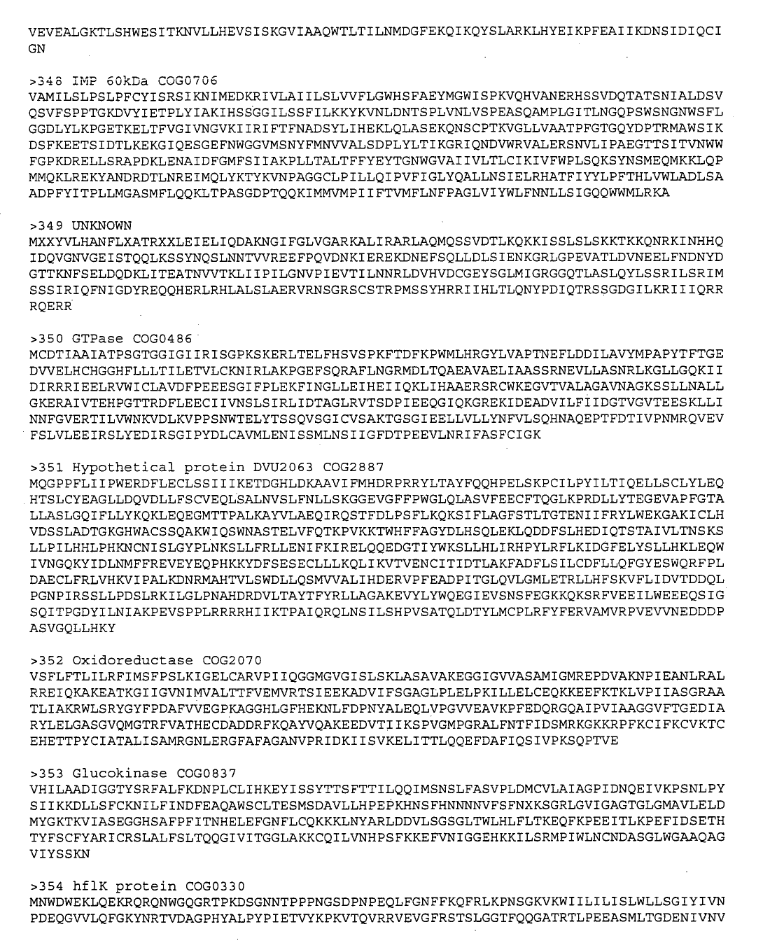

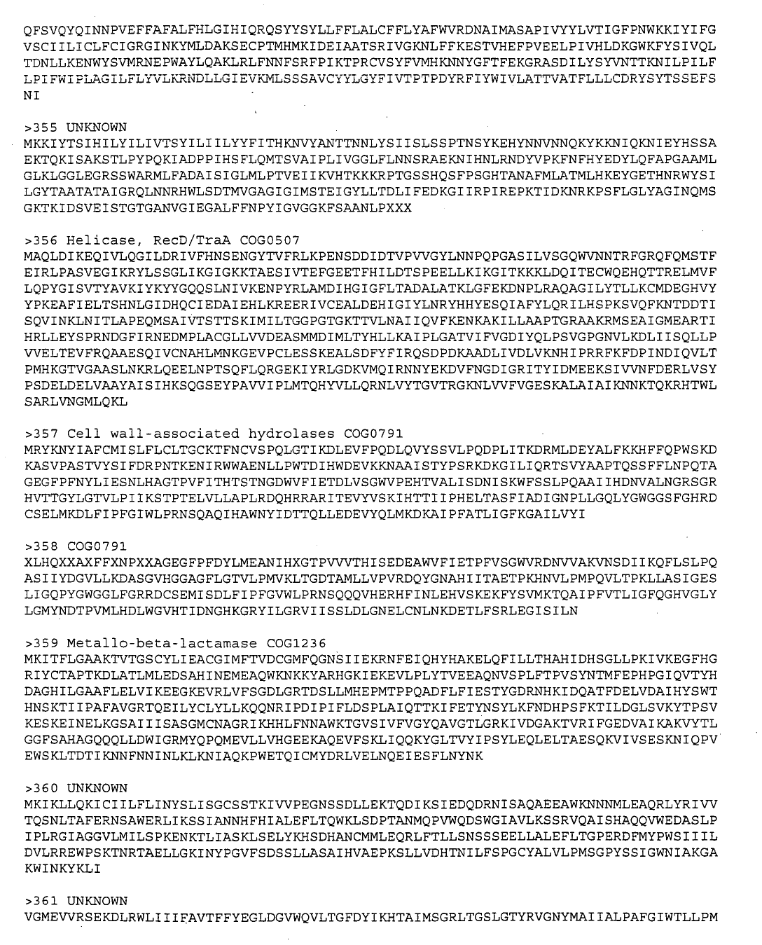

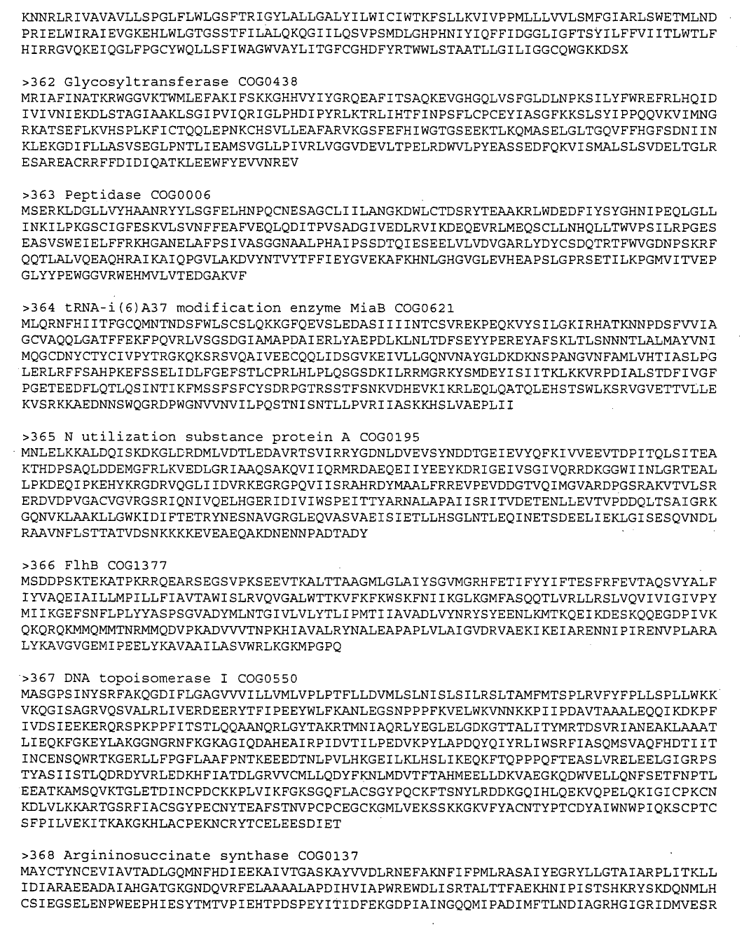

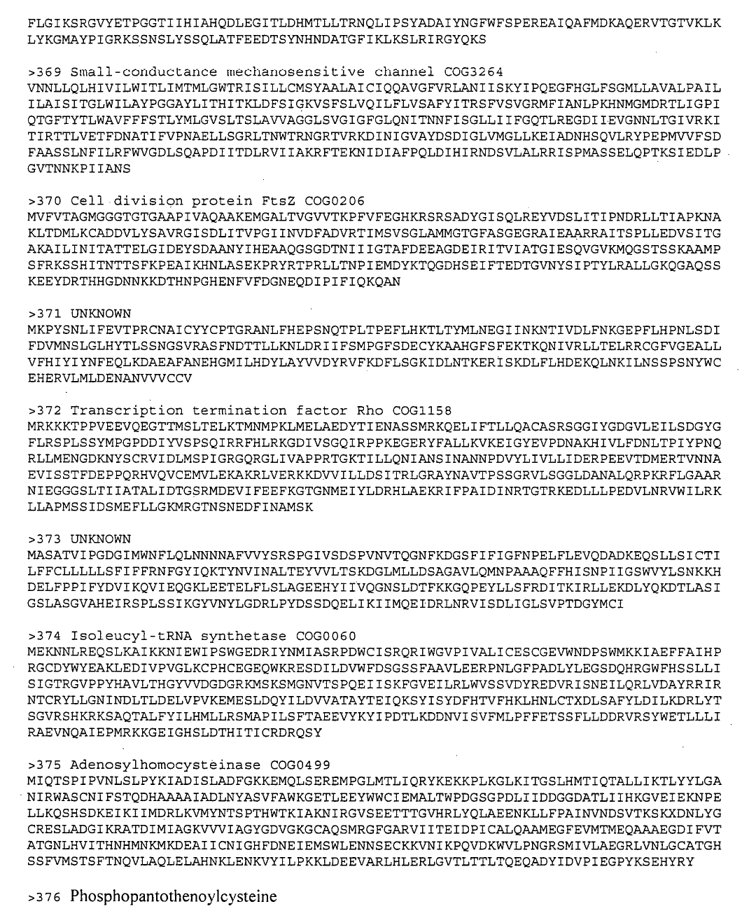

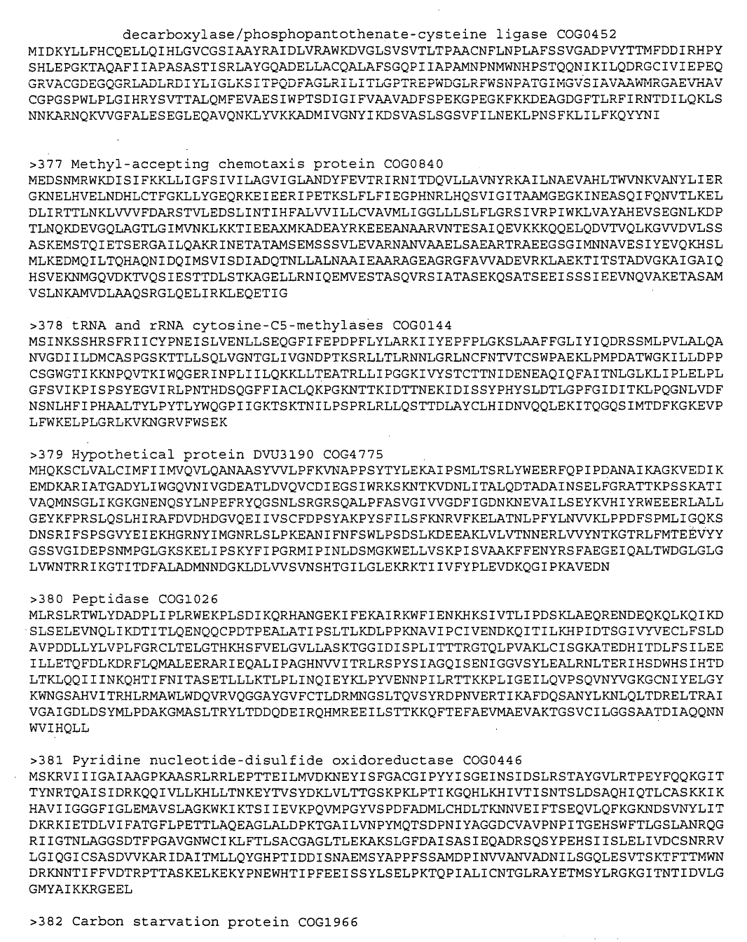

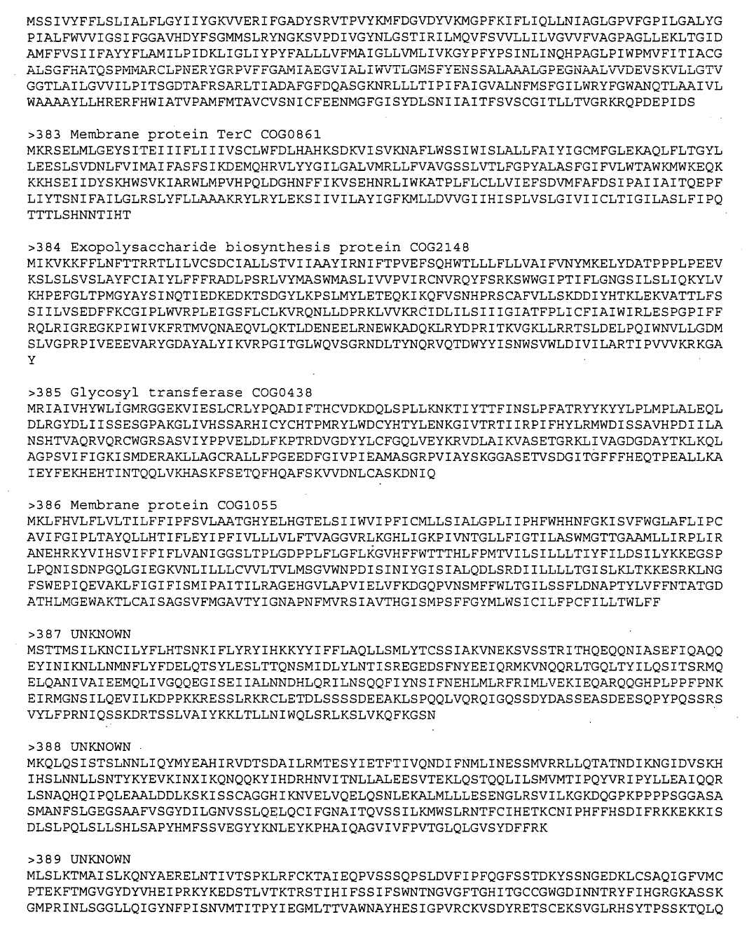

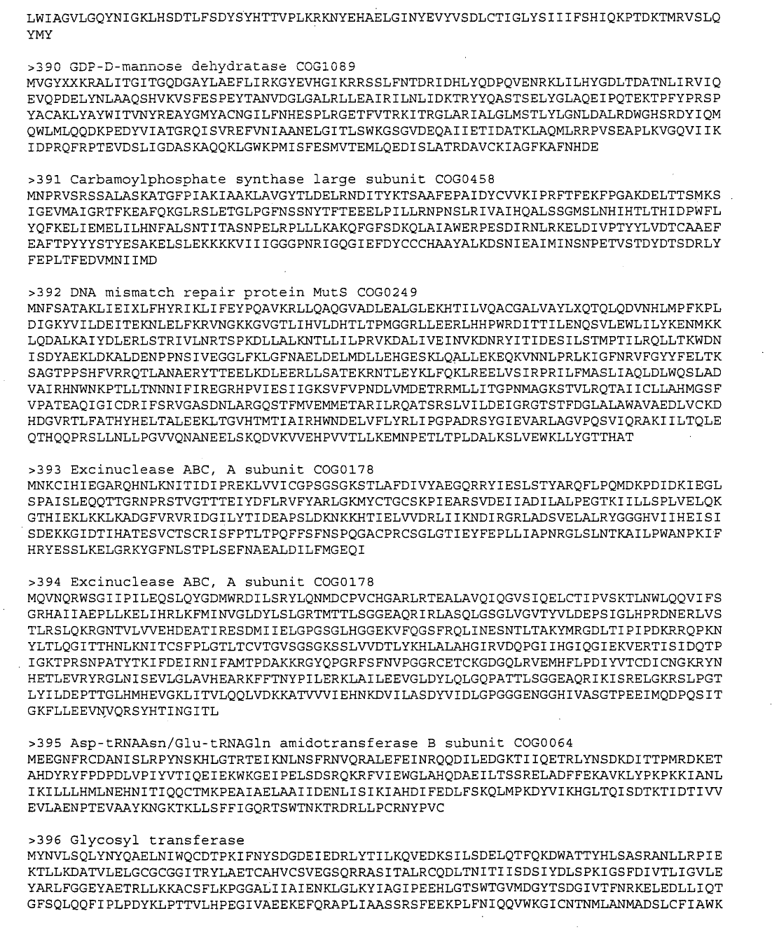

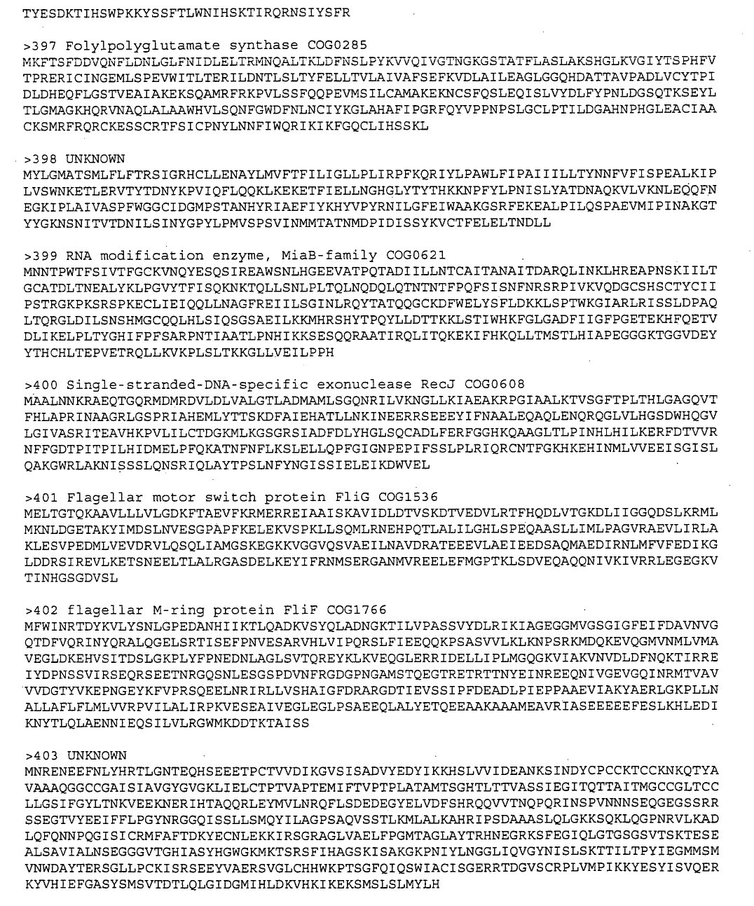

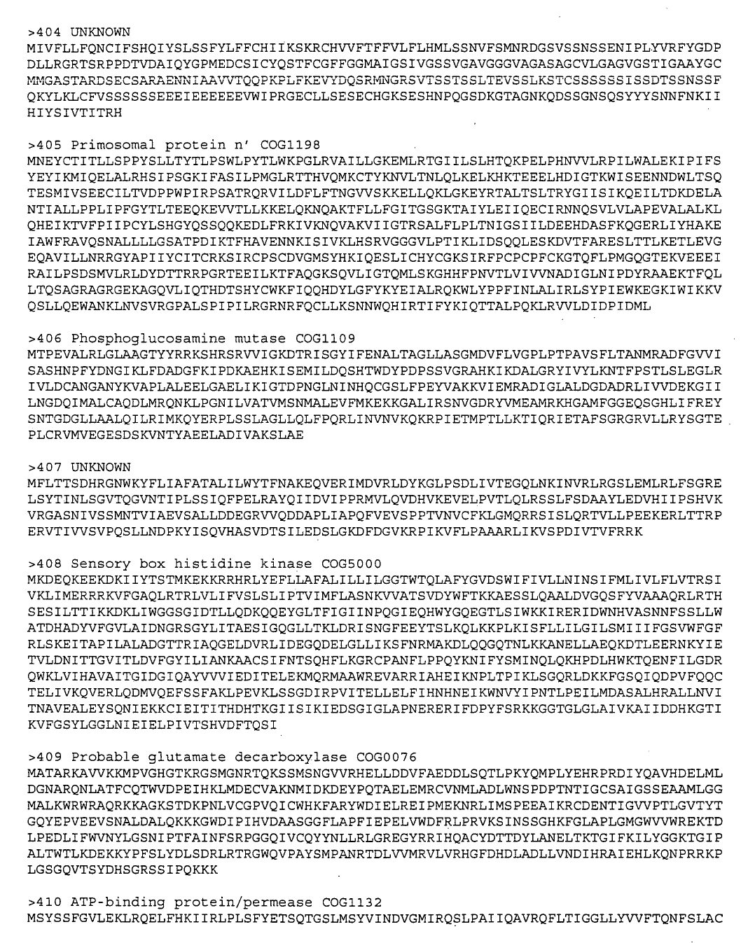

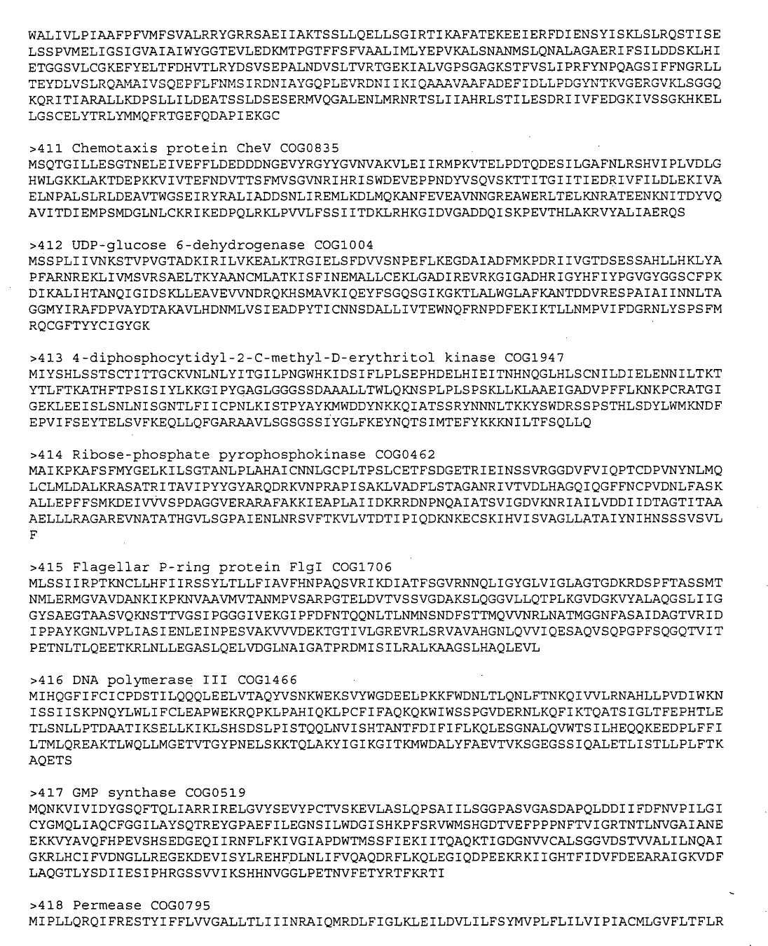

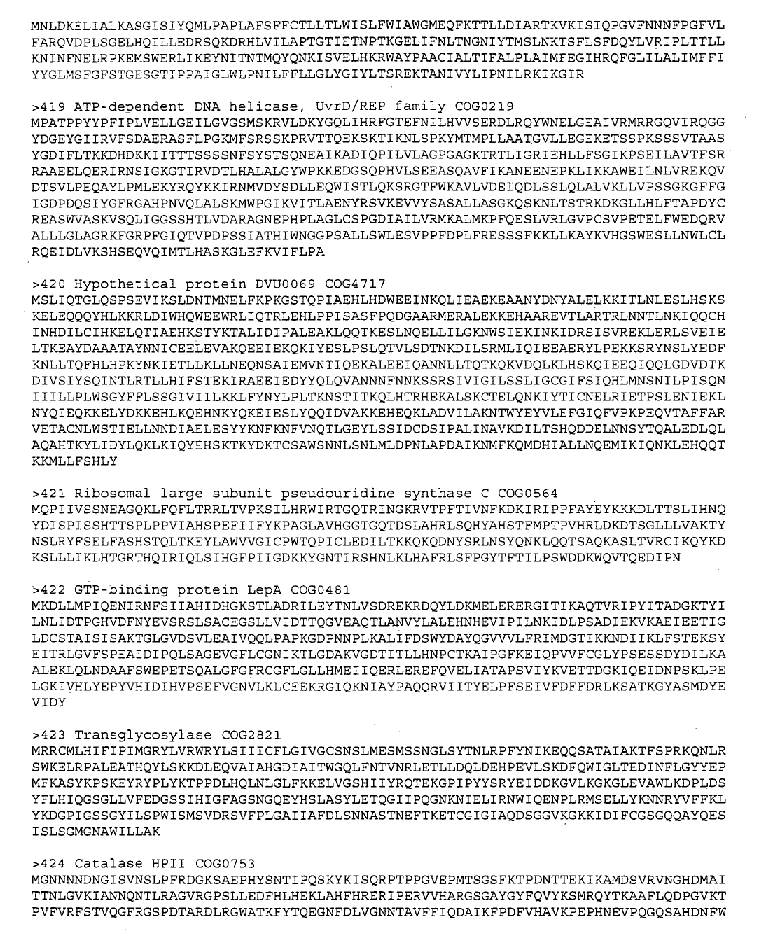

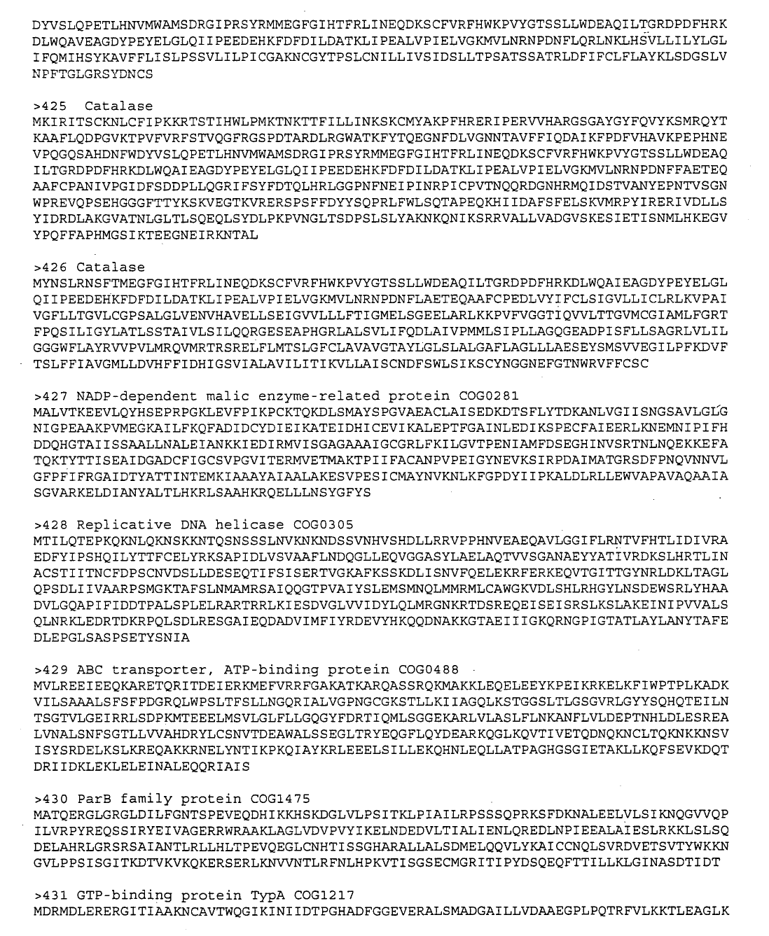

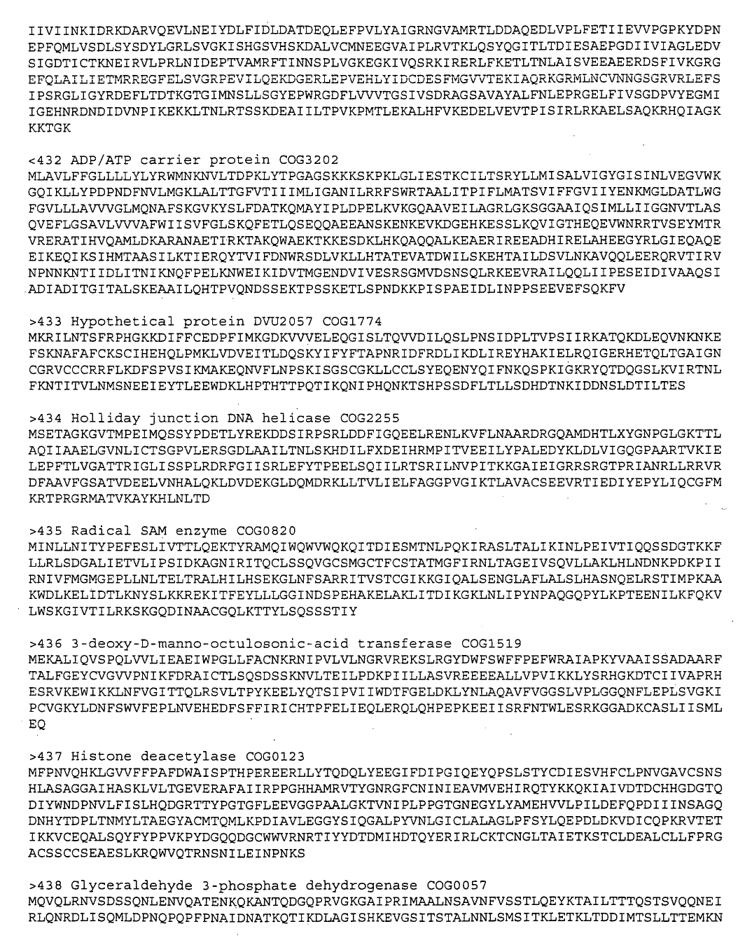

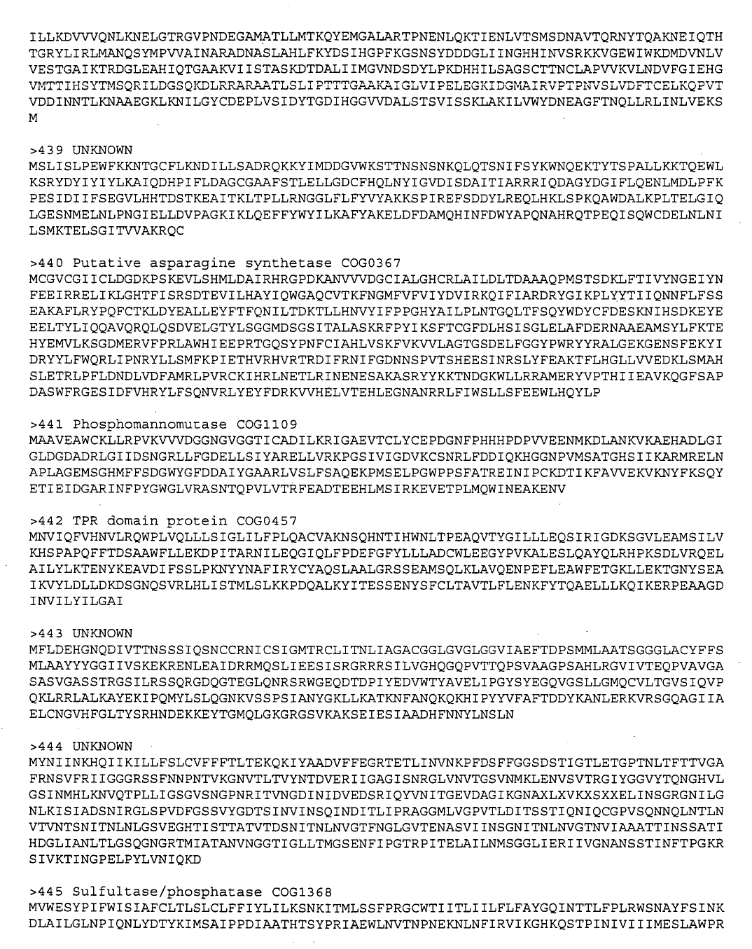

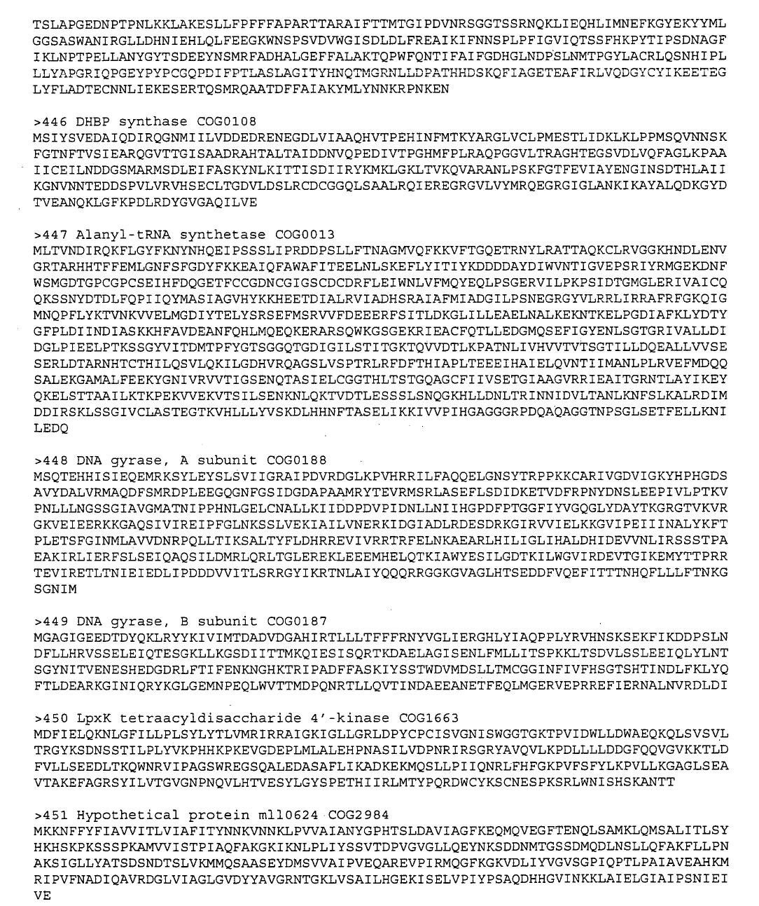

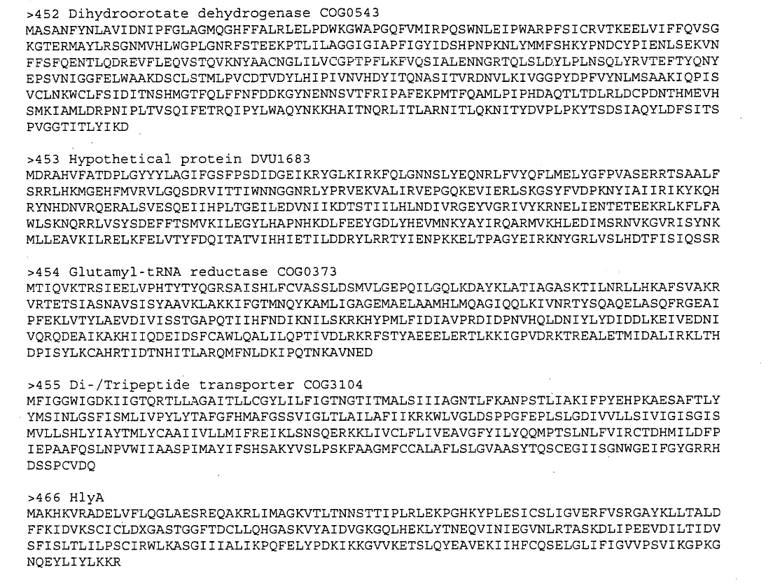

FIG. 3 shows the Coomassie stained gel picture of total HlyA and Omp85-like protein samples as well as partially purified HlyA protein from IMAC fraction A12 from the previous example. The result from the conjugate only blot is provided in FIG. 4A; the result of the Swine anti-Lawsonia blot may be seen as FIG. 4B; and the result of the negative control blot can be seen as FIG. 4C. Very little banding was observed in the conjugate-only blot. There was some background reactivity of antibodies in the swine serums to E. coli proteins. The reactivity of the HlyA and Omp85-like proteins was much more intense than in the swine convalescent serum as opposed to that from the strict control. The convalescent serum also reacted to the unique high molecular weight banding in the HlyA samples. Although HlyA and Omp85-like bands can be observed in the strict control Western blot, they are not as intense. Based on this data, it appears that the infection/challenge of pigs with Lawsonia results in the production of antibodies against the HlyA and Omp85-like proteins, which indicates that these may be useful proteins for a vaccine.

### EXAMPLE 4

This example describes the formation of a vaccine. Generally, any one of or a combination of a proteins selected from the group consisting of:
1) a polypeptide comprising a sequence selected from the group consisting of SEQ ID Nos.: 1-455, SEQ ID No 466, or the polypeptide encoded by SEQ ID No: 456, SEQ ID No: 457 or SEQ ID No: 466;
2) any polypeptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of 1);
3) any immunogenic portion of the polypeptides of 1) and/or 2)
4) the immunogenic portion of 3), comprising at least 300, 290, 280, 270, 260, 250, 240, 230, 220, 210, 200, 190, 180, 170, 160, 150, 140, 130, 120, 110, 100, 90, 80, 70, 60, 50, 45, 40, 35, 30, 25, 20, 18, 15, 13, 10, or most preferably 9 contiguous amino acids included in the sequences of SEQ ID No: 1-455, SEQ ID No: 456, or the amino acid sequence encoded by SEQ ID No: 457 or SEQ ID. No: 466; and/or
5) a polypeptide that is encoded by a DNA that codes for a peptide comprising the sequence of SEQ ID No: 1-455 or SEQ ID No: 466, are provided for use as the antigenic portion of a vaccine.

Veterinary-acceptable carriers, such as adjuvants, diluents, and the like will be added to the vaccine and the vaccine will be administered in any conventional manner.

We claim:

1. An isolated or recombinant immunological protein that is selected from the group of:
1) a polypeptide comprising a sequence selected from the group consisting of SEQ ID Nos. 1-455, SEQ ID No. 466, or the polypeptide encoded by SEQ ID No. 456 or SEQ ID No. 457;
2) any polypeptide that has at least 85% sequence homology with the polypeptide of 1);
3) any immunogenic portion of the polypeptides of 1) and/or 2)
4) the immunogenic portion of 3), comprising at least 9 contiguous amino acids included in the sequences of SEQ ID No. 1-455, SEQ ID No. 466, or the amino acid sequence encoded by SEQ ID No. 456 or SEQ ID No. 457; and
5) a polypeptide that is encoded by a DNA that codes for a peptide comprising the sequence of any one of SEQ ID Nos. 1-455 or SEQ ID No. 466.

2. An isolated or recombinant DNA molecule that includes a nucleotide sequence which encodes for an immunological protein selected from the group consisting of:
1) a polypeptide comprising a sequence selected from the group consisting of SEQ ID Nos. 1-455, SEQ ID No. 466, or the polypeptide encoded by SEQ ID No. 456 or SEQ ID No. 457;
2) any polypeptide that has at least 85% sequence homology with the polypeptide of 1);
3) any immunogenic portion of the polypeptides of 1) and/or 2)
4) the immunogenic portion of 3), comprising at least 9 contiguous amino acids included in the sequences of SEQ ID No. 1-455, SEQ ID No. 466, or the amino acid sequence encoded by SEQ ID No. 456 or SEQ ID No. 457; and
5) a polypeptide that is encoded by a DNA that codes for a peptide comprising the sequence of any one of SEQ ID Nos. 1-455 or SEQ ID No. 466.

3. A vector comprising a DNA molecule according to claim 2 or the protein according to claim 1.

4. An immunological composition comprising the DNA molecule according to claim 2 or the protein according to claim 1.

5. The composition of claim 4, further comprising a veterinary acceptable carrier.

6. A method for the prevention or treatment of an animal against Lawsonia intracellularis infections comprising the step of inoculating said animal with a product selected from the group consisting of an immunological protein according to claim 1, the DNA molecule according to claim 2, the vector according to claim 3, or the immunological composition according to claim 4.

7. The method of claim 6, said inoculation occurring intramuscularly, orally, or intranasally.

8. The use of an immunological protein according to claim 1, the DNA molecule according to claim 2, the vector according to claim 3, or the immunological composition according to claim 4 for the preparation of a medicament for the prevention or treatment of an animal against Lawsonia intracellularis infections.

9. A method for the detection of antibodies in a sample, comprising the steps:
1) Contacting a sample comprising antibodies against Lawsonia intracellularis bacteria with a protein according to claim 1;
2) Incubating the mixture of 1) under conditions which allow the protein according to claim 1 to bind to the Lawsonia specific antibodies of the sample and to generate a complex of Lawsonia specific antibody and the protein according to claim 1; and
3) Detecting the presence of the complex of 2).
